(19) United States
(12) Patent Application Publication
(10) Pub. No.: US 2004/0235766 A1
(43) Pub. Date: Nov. 25, 2004

(54) System for discovery of agents that block yersinia pestis and pseudomonas aeruginosa dna replication

(76) Inventors: James M. Bullard, Longmont, CO (US); Nebojsa Janjic, Boulder, CO (US); Charles S. McHenry, Denver, CO (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/476,597

(22) PCT Filed: May 14, 2002
(86) PCT No.: PCT/US02/15111

(30) Foreign Application Priority Data
May 14, 2001 (US) 60290725
Nov. 5, 2001 (US) 60332644

(51) Int. Cl. A61K 48/00; C12Q 1/68

(52) U.S. Cl. 514/44; 435/6

(57) ABSTRACT

Y. pestis and P. aeruginosa nucleic acid molecules encoding dnaE, holA, holB, holC, holD, holE, dnaX, dnaN, SSB, dnaG, dnaQ, proteins are provided. The encoded proteins are also provided. The nucleic acid molecules and proteins are useful for reconstituting replicases and polymerases for sequencing, amplification, and screening for compounds which modulate the function of the polyemersase or replicase.

### STATEMENT REGARDING FEDERALLY-SPONSORED RESEARCH AND DEVELOPMENT

Statement under MPEP 310. The U.S. Government has a paid-up license in this invention and the right in limited circumstances to require the patent owner to license others on reasonable terms as provided for by the terms of SBIR Grant 1 R43 GM64854-01 awarded by The National Institutes of Health (NIH), and Defense Advanced Research Projects Agency (DARPA) Grant N65236-01-1-7402.

Part of the work performed during development of this invention utilized U.S. Government funds. The U.S. Government has certain rights in this invention.

### FIELD OF THE INVENTION

The present invention relates to gene and amino acid sequences encoding DNA polymerase III holoenzyme subunits and structural genes from Yersinia pestis and Pseudomonas aeruginosa, as well as the assembly of a complete chromosomal DNA replication elongation system for the respective organisms. The present invention also provides antibodies and other reagents useful to identify DNA polymerase III molecules.

### BACKGROUND OF THE INVENTION

DNA Replication. Like many other complex mechanisms of macromolecular synthesis, the fundamental mechanisms of DNA replication have been conserved throughout biology. The chemistry and direction of synthesis, the requirement for RNA primers, the mechanisms of semi-discontinuous replication with Okazaki fragments on the lagging strand and the need for well-defined origins are shared (Kornberg, A. and Baker, T. A. (1992) DNA Replication, 2 Ed., WH Freeman and Company, New York). This reference, and all other references cited is herein are incorported by reference in their entirety. The basic features of the replicative apparatus are also shared. All replication systems consist of a replicative polymerase that is distinguished from others primarily by its capability of participating in specific protein-protein interactions with the rest of the replicative apparatus. All cells, both eukaryotic and prokaryotic, contain multiple DNA polymerases. Yet, only a subset of these polymerases can function as the replicative catalytic subunit. In eukaryotes, it is most likely that the δ polymerase is the major replicative polymerase (Nethanel, T. and Kaufmann, G. (1990) J Virol 64, 5912-5918), whereas, in E. coli, the α subunit of DNA polymerase III serves as the polymerization subunit. Polymerase III subunits are named both as gene products and by individual Greek letters: α (DnaE), polymerase catalytic subunit; β (DnaN), sliding clamp processivity factor; DnaX (τ (and in E. coli and T. thermophilus γ)); ATPase subunit of DnaX complex; δ (HolA) and δ′ (HolB), essential auxiliary subunits of DnaX complex. These two polymerases are of different classes and provide no detectable homology at the protein sequence level. Another key replication system component is the so-called sliding clamp that confers high processivity on the replication system (processivity is defined as the number of nucleotides inserted per template association-catalysis-dissociation event). It consists of a bracelet-shaped molecule that clamps around DNA, permitting it to rapidly slide down DNA, but not to dissociate. The clamp contacts the polymerase by protein-protein interactions and prevents it from falling off of the template, ensuring high processivity. Prokaryotic and eukaryotic sliding clamps are β and PCNA, respectively. The crystal structures of yeast PCNA and E. coli β are nearly superimposable (Kong, X. P., Onrust, R., O'Donnell, M., and Kuriyan, J. (1992) Cell 69, 425-43717; Krishna, T. S., Kong, X. P., Gary, S., Burgers, P. M., and Kuriyan, J. (1994) Cell 79, 1233-124318); yet, the relationship between the two is only at the structural and functional level. No homology is apparent between PCNA and β at the protein sequence level. In both bacteria and eukaryotes, a 5-protein complex is responsible for transferring the sliding clamp onto a primer-terminus in an ATP-dependent reaction (Lee, S. H., Kwong, A. D., Pan, Z. Q., and Hurwitz, J. (1991) J Biol Chem 266, 594-602; Bunz, F., Kobayashi, R., and Stillman, B. (1993) Proc Natl Acad Sci U S A 90, 11014-11018). Some of the subunits exhibit recognizable sequence homology between eukaryotes and bacteria, but primarily in the conserved ATP binding N-terminal domain. Even there, sufficient differences exist that it should be possible to optimize lead compounds to be bacteria-specific (Carter, J. R., Franden, M. A., Aebersold, R., and McHenry, C. S. (1993) J Bacteriol 175, 3812-3822; O'Donnell, M., Onrust, R., Dean, F. B., Chen, M., and Hurwitz, J. (1993) Nucleic Acids Res 21, 1-3; Bruck, I. and O'Donnell, M. (2000) J Biol Chem 275, 28971-28983).

The DNA polymerase III holoenzyme is the replicative polymerase of E. coli, responsible for synthesis of the majority of the chromosome (for a review, see Kelman, Z. and O'Donnell, M. (1995) Annu Rev Biochem 64, 171-200). The replicative role of the enzyme has been established both by biochemical and genetic criteria. Holoenzyme was biochemically defined and purified using natural chromosomal assays. Only the holoenzyme form of DNA polymerase III efficiently replicates single-stranded bacteriophages in vitro in the presence of other known replicative proteins (Wickner, W. and Kornberg, A. (1973) Proc Natl Acad Sci U S A 70, 3679-3683; Hurwitz, J. and Wickner, S. (1974) Proc Natl Acad Sci U S A 71, 6-10; McHenry, C. S. and Kornberg, A. (1977) J Biol Chem 252, 6478-6484). Only the holoenzyme functions in the replication of bacteriophage λ plasmids, bacteriophage Mu and molecules containing the E. coli replicative origin, oriC (Wold, M. S., Mallory, J. B., Roberts, J. D., Lebowitz, J. H., and McMacken, R. (1982) Proc Natl Acad Sci U S A 79, 6176-6180; Kaguni, J. M., Fuller, R. S., and Kornberg, A. (1982) Nature 296, 623-627; Katayama, T., Kubota, T., Kurokawa, K., Crooke, E., and Sekimizu, K. (1998) Cell 94, 61-71; Jones, J. M. and Nakai, H. (1997) EMBO J 16, 6886-6895). The holoenzyme contains 10 subunits: α,τ,γ,β,δ,δ′,ε,ψ,χ and θ of 129,900, 71,000, 47,400, 40,600, 38,700, 36,900, 26,900, 16,600, 15,000 and 8,800 daltons, respectively.

Genetic studies also support assignment of the major replicative role to the pol III holoenzyme and further validate it as a target for anti-bacterial action. Temperature-sensitive mutations in dnaE, the structural gene for the a catalytic subunit, are conditionally lethal (Gefter, M. L., Hirota, Y., Kornberg, T., Wechsler, J. A., and Barnoux, C. (1971) Proc Natl Acad Sci U S A 68, 3150-3153). Similarly, temperature-sensitive, conditionally-lethal mutations have been isolated for the dnaN, dnaX and dnaQ genes that encode the β processivity factor, the DnaX protein and the ε proofreading exonuclease, respectively (Sakakibara, Y. and Mizukami, T. (1980) Mol Gen Genet 178, 541-553; Chu, H., Malone, M. M., Haldenwang, W. G., and Walker, J. R. (1977) J Bacteriol 132, 151-158; Henson, J. M., Chu, H., Irwin, C. A., and Walker, J. R. (1979) Genetics 92, 1041-1059; Horiuchi, T., Maki, H., and Sekiguchi, M. (1978) Mol Gen Genet 163, 277-283). The structural genes for the final five holoenzyme subunits were identified by a reverse genetics approach (Carter, et al., ibid.; Carter, J. R., Franden, M. A., Aebersold, R., and McHenry, C. S. (1992) J Bacteriol 174, 7013-7025; Carter, J. R., Franden, M. A., Lippincott, J. A., and McHenry, C. S. (1993) Mol Gen Genet 241, 399-408; Carter, J. R., Franden, M. A., Aebersold, R., Kim, D. R., and McHenry, C. S. (1993) Nucleic Acids Res 21, 3281-3286; Carter, J. R., Franden, M. A., Aebersold, R., and McHenry, C. S. (1993) J Bacteriol 175, 5604-5610; Dong, Z., Onrust, R., Skangalis, M., and O'Donnell, M. (1993) J Biol Chem 268, 11758-11765; Xiao, H., Crombie, R., Dong, Z., Onrust, R., and O'Donnell, M. (1993) J Biol Chem 268, 11773-11778). These were named these holA-E for the δ,δ′,χ, ψ and θ genes, respectively. Recently, knockout mutants of holA and B have shown that both δ and δ′ are essential for cell viability (Song, M.-S., Pham, P. T., Olson, M., Carter, J. R., Franden, M. A., Schaaper, R. M., and McHenry, C. S. (2001) J Biol Chem, 276, 35165-35175), validating them along with dnaE, dnaX, dnaQ and dnaN as targets for antibacterial development.

Of the five DNA polymerases identified in E. coli, only the pol III holoenzyme appears to play a major replicative role. What are the special features of the pol III holoenzyme that confer its unique role in replication? Work to date suggests that the rapid elongation rate, high processivity, ability to utilize a long single-stranded template coated with the single-stranded DNA binding protein, resistance to physiological levels of salt and ability to interact with other proteins of the replicative apparatus are all critical to its unique functions. In contrast, other non-replicative polymerases such as DNA polymerase I cannot substitute effectively for replicative polymerases either in vitro or in vivo (Kornberg & Baker, ibid.). This is because of their low processivity, their corresponding slow reaction rate, and their inability to interact with other replication proteins to enable catalysis of a coordinated reaction at the replication fork.

DNA polymerase III holoenzyme is conveniently assayed by conversion of M13Gori single-stranded DNA to the duplex replicative form (FIG. 1). This assay recapitulates all of the interactions required for Okazaki fragment synthesis on the lagging strand and for elongation of the leading strand at the bacterial replication fork. Only the DnaB helicase, required for separating two strands of DNA in advance of replication is missing since it is not required for G4 origin function. This assay has been adapted to a high-throughput format by monitoring the increase of fluorescence that occurs upon addition of an intercalating fluorophore to the duplex DNA product (Seville, M., West, A. B., Cull, M. G., and McHenry, C. S. (1996) Biotechniques 21, 664-672).

Multiple DNA polymerase III forms. The DNA polymerase III holoenzyme can be biochemically resolved into a series of successively simpler forms. DNA polymerase III core contains the a catalytic subunit complexed tightly to ε (proofreading subunit with 3′→5′ exonuclease activity) and θ. DNA polymerase III′ contains core +τ. DNA polymerase III* contains pol III′+the DnaX γ complex (γ,δδ′,χψ). Holoenzyme is composed of pol III*+β.

Processivity. Studies of the processivities of the multiple polymerase III forms have revealed individual contributions of subunits (Fay, P. J., Johanson, K. O., McHenry, C. S., and Bambara, R. A. (1981) J Biol Chem 256, 976-983; Fay, P. J., Johanson, K. O., McHenry, C. S., and Bambara, R. A. (1982) J Biol Chem 257, 5692-5699). The multiple forms of DNA polymerase III exhibit strikingly different processivities. The core pol III has a low processivity (ca. 10 bases) in low ionic strength that decreases to being completely distributive (processivity=1) under more physiological conditions. The holoenzyme exhibits a processivity orders of magnitude greater than any of its subassemblies. In carefully controlled experiments with single-stranded phage, the entire template (about 8000 nucleotides) is synthesized in a single processive event in under 15 s at 30° C. (Johanson, K. O. and McHenry, C. S. (1982) J Biol Chem 257, 12310-12315). For coupled replication fork systems where the holoenzyme acts with primosomal components, processivities of 150-500 kb have been directly observed (Wu, C. A., Zechner, E. L., Hughes Jr, A. J., Franden, M. A., McHenry, C. S., and Marians, K. J. (1992) J Biol Chem 267, 4064-4073). These products are synthesized at rates of 500-700 nt/s. Thus, a progression in processivities that parallels the structural complexity of the corresponding enzyme form is observed. For comparative purposes, the processivities of “repair-type” polymerases of the bacterial DNA polymerase I class are typically 15-50 bases (Bambara, R. A., Uyemura, D., and Choi, T. (1978) J Biol Chem 253, 413-423).

Structure of the β Sliding Clamp. The X-ray crystallographic structure of β, solved by Kuriyan, O'Donnell and coworkers (Kong, et al., ibid.), provides a simple and elegant explanation for its function. The β dimer forms a bracelet-like structure, presumably with DNA passing through the central hole, permitting it to slide down DNA rapidly but preventing it from readily dissociating. Protein-protein contacts between β and other components of the replicative complex tether the polymerase to the DNA, increasing its processivity. A tightly clasped bracelet would not be expected to readily associate with DNA. This explains the need for an energy-dependent clamp-setting complex, the DnaX-complex, to recognize the primer terminus and open and close the β-bracelet around DNA.

Initiation Complex Formation. To achieve high processivity, the holoenzyme requires ATP (or dATP) and primed DNA to form a stable initiation complex (Fay, et al. 1981, ibid.). Initiation complexes can be isolated by gel filtration and, upon addition of dNTPs, form a complete RFII (replicative form II, a duplex circle containing one nick at the site where replication is completed) in 10-15 seconds without ever dissociating (Wickner & Kornber, ibid.; Hurwitz & Wickner, ibid.; Johanson, K. O. and McHenry, C. S. (1980) J Biol Chem 255, 10984-10990). Initiation complex formation can be monitored experimentally as a conversion of replicative activity to anti-β IgG resistance (Carter, et al., 1992, ibid.; Johanson & McHenry, ibid. 1980). β participates in elongation; antibody resistance arises from β's immersion in the complex, sterically precluding antibody attachment.

DnaX Complex: The Apparatus that Sets the β Sliding Clamp onto Primed DNA. The DnaX protein contains a consensus ATP binding site near its amino-terminus (Yin, K. C., Blinkowa, A., and Walker, J. R. (1986) Nucleic Acids Res 14, 6541-6549) that is used to bind and hydrolyze ATP, in concert with δ-δ′-χ-ψ, setting the β processivity clamp on the primer-terminus. DnaX binds ATP with a dissociation constant of ca. 2 μM (Tsuchihashi, Z. and Kornberg, A. (1989) J Biol Chem 264, 17790-17795) and is a DNA-dependent ATPase (Ibid.; Lee, S. H. and Walker, J. R. (1987) Proc Natl Acad Sci U S A 84, 2713-2717). The primer-terminus appears to be the most active effector of the ATPase (Onrust, R., Stukenberg, P. T., and O'Donnell, M. (1991) J Biol Chem 266, 21681-21686). The dnaX gene expresses two related proteins, γ and τ, where γ represents the amino-terminal 5/7 of τ (Flower, A. M. and McHenry, C. S. (1990) Proc Natl Acad Sci U S A 87, 3713-3717). DnaX (τ) binds the α subunit DNA polymerase III core and causes it to dimerize, forming the dimeric scaffold upon which other auxiliary proteins can assemble to form a dimeric replicative complex. In vitro, the τ DnaX subunit can readily form a “τ-complex” (τ-ψ-χ-δ-δ′) that functions to load β onto primed DNA (Dallmann, H. G. and McHenry, C. S. (1995) J Biol Chem 270, 29563-29569; Onrust, R., Finkelstein, J., Naktinis, V., Turner, J., Fang, L., and O'Donnell, M. (1995) J Biol Chem 270, 13348-13357; Dallmann, H. G., Thimmig, R. L., and McHenry, C. S. (1995) J Biol Chem 270, 29555-29562). The stoichiometry of the DnaX complexes has been determined and it has been found that it has 3 copies of the DnaX protein and 1 each of the ancillary subunits (DnaX₃δ₁δ′₁χ₁ψ₁) (Pritchard, A., Dallmann, G., Glover, B., and McHenry, C. (2000) EMBO J 19, 6536-6545). The form of DnaX complex contained within native holoenzyme is τ₂γδ₁δ′₁χ₁ψ₁.

Structure of the DNA polymerase III holoenzyme. FIG. 2 illustrates the current understanding of holoenzyme subunit-subunit interactions. α and ε form an isolable complex upon mixing (Maki, H. and Kornberg, A. (1987) Proc Natl Acad Sci U S A 84, 4389-4392). Mutations in the structural gene for ε have also been found that suppress dnaE (α) mutations (Maurer, R., Osmond, B. C., and Botstein, D. (1984) Genetics 108, 25-38). Suppressor mutations most likely arise through modification of a subunit that interacts directly with the suppressed mutant gene product. Pol III core (αεθ) is isolable (McHenry, C. S. and Crow, W. (1979) J Biol Chem 254, 1748-1753). DnaX (τ) can be isolated in a complex with pol III core (McHenry, C. S. (1982) J Biol Chem 257,2657-2663). DnaX in the presence of δ and δ′ can transfer β to primed DNA to form a preinitiation complex (Bryan, S., Hagenessee, M., and Moses, R. E. (1990) DNA Polymerase III is Required for Mutagenesis. In Moses, R. and Summers, W., editors. DNA Replication and Mutagenesis, American Society for Microbiology, Washington, D.C.). Suppressor data indicate an interaction between β and α (Kuwabara, N. and Uchida, H. (1981) Proc Natl Acad Sci U S A 78, 5764-5767), a notion supported by the ability of β to interact with and increase the processivity of core pol III (Laduca, R. J., Crute, J. J., McHenry, C. S., and Bambara, R. A. (1986) J Biol Chem 261, 7550-7557) and the observation of an interaction between β and the carboxyl-terminal domain of the α catalytic subunit (Kim, D. R. and McHenry, C. S. (1996) J Biol Chem 271, 20699-20704). Genetic evidence for a-a interaction and for the dimeric nature of pol III holoenzyme was obtained through interallelic complementation between dnaE₁₀₂₆ and dnaE₄₈₆ or dna E₅₁₁ (Byran, et al., ibid.)—if this interaction occurs, it must be weak and require the presence of other subunits, since no α-α interaction is seen in vitro. χ and ψ have been isolated in a complex with DnaX (Olson, M. W., Dallmann, H. G., and McHenry, C. S. (1995) J Biol Chem 270, 29570-29577; O'Donnell, M. and Studwell, P. S. (1990) J Biol Chem 265, 1179-1187). δ and δ′ can interact weakly by themselves in solution and together can interact with DnaX (Dallmann, & McHenry, ibid.; Olson, et al., ibid.; Onrust, R. and O'Donnell, M. (1993) J Biol Chem 268, 11766-11772). ψ and δ′ are apparently the subunits that interact directly with DnaX (Onrust, et al., 1995, ibid.). A direct δ-β interaction has been detected (Naktinis, V., Onrust, R., Fang, L., and O'Donnell, M. (1995) J Biol Chem 270, 13358-13365). There are three copies of DnaX protein in holoenzyme (Pritchard, et al., ibid.).

Subunit Homology Among Species. As indicated in Table I, some of the basic mechanistic features of cellular replication systems are employed by both prokaryotes and eukaryotes.

TABLE I
Relationships of the Components of Replicative Enzymes

| Component/ | | | |
|---|---|---|---|
| Function | E. coli | Phage T4 | Eukaryotes |
| DNA | α | Gene 43 | polymerase |
| polymerase | | | δ (and |
| | | | possibly ε) |
| Sliding Clamp | β | Gene 45 protein | PCNA |
| Clamp loading | DnaX, δ, δ′, χ, ψ | Gene 44/62 | Activator |
| complex | | complex | 1 (RFC) |
| Single-stranded | SSB | Gene 32 protein | RFA |
| DNA | | | |
| binding protein | | | |
| Primer generation | DnaG primase | Gene 61 protein | primase- |
| | | | α polymerase |
| | | | complex |

Nevertheless, there is also significant sequence and functional divergence. Within the eleven genes encoding the core components required for synthesis on single-stranded DNA, only dnaX (encoding γ and τ) and holB (encoding δ′) share significant homology with the genes of eukaryotic cells (Carter, et al., ibid. (1993) J Bacteriol 175, 3812-3822; O'Donnell, 1993, ibid.). Even then, the homology is confined to the core ATP-binding site. Natural inhibitory proteins (e.g., p21) that bind to and regulate the eukaryotic regulatory apparatus (Waga, S., Hannon, G. J., Beach, D., and Stillman. (1994) Nature 369, 574-578; Gulbis, J. M., Kelman, Z., Hurwitz, J., O'Donnell, M., and Kuriyan, J. (1996) Cell 87, 297-306) do not affect the corresponding prokaryotic proteins.

Thus, it is expected that the more complex multi-enzyme system approach will be much more efficient in permitting the identification of novel antibacterials than would be possible by simply screening against those few proteins that exhibit an intrinsic activity by themselves. There remains a need for a multi-enzyme assay that will detect inhibitors that prevent changing protein-protein interaction during the holoenzyme-catalyzed reaction and permit their detection, by a simple functional read-out.

Bacteria and Antibacterial Drugs. Bacterial infections continue to represent a major worldwide health hazard (Overcoming Antimicrobial Resistance (2000). World Health Organization, Report on Infectious Diseases (WHO/CDS/2000.2)). Infections range from the relatively innocuous, such as skin rashes and common ear infections in infants, to the very serious and potentially lethal infections in immune-compromised patients. We are increasingly confronted with drug-resistant hospital-acquired and community infections. With the recent emergence of numerous, clinically important, drug-resistant bacteria including Staphylococcus aureus, Streptococcus pneumoniae, Enterococcus faecalis, Mycobacterium tuberculosis, and Pseudomonas aeruginosa, an emergency is becoming apparent.

Classes of Antibacterials and Mechanism of Resistance. Antibacterials kill bacteria by interfering with major processes of cellular function that are essential for survival. The β-lactams (penicillins and cephalosporins) and the glycopeptides (vancomycin and teicoplanin) inhibit synthesis of the cell wall. Macrolides (erythromycin, clarithromycin, and azithromycin), clindamycin, chloramphenicol, aminoglycosides (streptomycin, gentamicin, and amikacin) and the tetracyclines inhibit protein synthesis. Also inhibiting protein synthesis is the newest class of antibacterials to be approved (linezolid) that are synthetic oxazolidinones. Rifampin inhibits RNA synthesis, the fluoroquinolones (such as ciprofloxacin) inhibit DNA synthesis indirectly by inhibiting the enzymes that maintain the topological state of DNA. Trimethoprim and the sulfonamides inhibit folate biosynthesis directly and DNA synthesis indirectly by depleting the pools of one of the required nucleotides (Chambers, H. F. and Sande, M. A. (1996) Antimicrobial Agents. Goodman & Gilman's The Pharmacological Basis of Therapeutics, McGraw-Hill, New York).

Resistance to antibacterials can occur when the target of a drug mutates so that it can still function, but is no longer blocked by the drug, or by mutation of efflux pumps or modification enzymes to broaden their specificity to a new drug. Because of the growth advantage this gives to the resistant cell and its progeny in the presence of the antibacterial, the resistant organisms quickly take over a population of bacteria. Resistance developed in one cell can be transferred to other bacteria in the population since bacteria have mechanisms for directly exchanging genetic material. In a recent congressional report, the General Accounting Office (GAO) has summarized the current and future public health burden resulting from drug-resistant bacteria (Antimicrobial Resistance (1999). General Accounting Office (GAO/RCED-99-132)). According to this report, the number of patients treated in a hospital setting for an infection with drug-resistant bacteria has doubled from 1994 to 1996 and again almost doubled from 1996 to 1997. Resistant strains can spread easily in environments such as hospitals or tertiary care facilities that have a sizeable population of immunosuppressed patients. The same GAO report also provides clear evidence that previously susceptible bacteria are increasingly becoming resistant and spreading around the world. Furthermore, the proportion of resistant bacteria within bacterial populations is on the rise. An especially frightening development is the appearance of bacterial strains that are resistant to all approved antibacterials. Recognizing the dramatic increase of drug-resistant bacteria, the Food and Drug Administration has recently issued a recommendation urging physicians to use antibacterials more judiciously and only when clinically necessary (FDA Advisory (2000). Federal Register 65 (182), 56511-56518).

Until recently, drug developers tended to respond to emerging, drug-resistant bacterial strains by simply modifying existing antibacterials. Indeed, most of the antibacterials in late-stage development are analogs of drugs already in clinical use. This strategy is becoming less effective since the basic mechanism for resistance to the antibacterial class is already widespread in nature. Further mutation of the resistant target to confer resistance on a new antibacterial of the same class can often occur by a single base change in the gene for the target. Thus, the utility of antibacterials presently in the development pipeline is expected to be finite and the health care industry is currently in severe need of novel antibacterials that are mechanistically distinct from existing drugs.

Pseudomonas aeruginosa. The need for more efficacious agents to treat P. aeruginosa infections is acute. This Gram-negative bacterium is omnipresent in the environment in large part due to its propensity to grow on many different surfaces including tissues from plants and animals, rocks, soil as well as synthetic materials such as contact lens, surgical instruments and catheters (Costerton, J. W., Stewart, P. S., and Greenberg, E. P. (1999) Science 284, 1318-1322). P. aeruginosa causes a wide range of infections including bacteremia in urinary tract infections, burn victims and patients on respirators. In hospitals, P. aeruginosa is responsible for about one-seventh of all infections with multidrug-resistant strains being increasingly common (Maschmeyer, G. and Braveny, I. (2000) Eur J Clin Microbiol Infect Dis 19, 915-925; Giamarellou, H. and Antoniadou, A. (2001) Med Clin North Am 85, 19-42). However, the most serious medical problem caused by P. aeruginosa are lung infections associated with cystic fibrosis (CF). In CF, the gene that encodes transmembrane regulator (CFTR) chloride channel is mutated leading to inefficient removal of salt from apical membranes of lung epithelial cells. Salt accumulation in the airway surface fluid leads to a variety of lung function abnormalities including salt-mediated inhibition of endogenous antibacterial peptides and proteins. This, in turn, leads to persistent P. aeruginosa infections that result in physical impairment of lung function caused by bacterial films as well as immune-mediated, inflammatory damage to the lung tissue provoked by antigenic determinants produced by the bacteria. Median life expectancy of CF patient is about 30 years. Prevention of initial lung tissue colonization by prophylactic treatment with antibiotics in younger CF patients is currently being examined as a possible clinical strategy of controlling lung damage (Johansen, H. K., Kovesi, T. A., Koch, C., Corey, M., Hoiby, N., and Levison, H. (1998) Pediatr Pulmonol 26, 89-96).

P. aeruginosa is intrinsically resistant to a wide range of antibiotics in part because its outer membrane lacks high-permeability porin proteins that serve to transport small molecules (including some antibiotics) into the periplasmic space (Nikaido, H. (1994) Science 264, 382-388). In addition, P. aeruginosa has several multidrug efflux systems that collectively serve to limit the concentration of antibiotics in the cell interior (Hancock, R. E. (1998) Clin Infect Dis 27 Suppl. 1, S93-S99; Westbrock-Wadman, S., Sherman, D. R., Hickey, M. J., Coulter, S. N., Zhu, Y. Q., Warrener, P., Nguyen, L. Y., Shawar, R. M., Folger, K. R., and Stover, C. K. (1999) Antimicrob. Agents Chemother. 43, 2975-2983). Additional efflux systems are apparent in a BLAST analysis of the recently completed P. aeruginosa genome (Stover, C. K., Pham, X. Q., Erwin, A. L., Mizoguchi, S. D., Warrener, P., Hickey, M. J., Brinkman, F. S. L., Hufnagle, W. O., Kowalik, D. J., Lagrou, M., Garber, R. L., Goltry, L., Tolentino, E., Westbrock-Wadman, S., Yuan, Y., Brody, L. L., Coulter, S. N., Folger, K. R., Kas, A., Larbig, K., Lim, R., Smith, K., Spencer, D., Wong, K.-S., Wu, Z., Paulsen, I. T., Reizer, J., Saier, M. H., Hancock, R. E. W., Lory, S., and Olson, M. V. (2000) Nature 406,959-964). One way to combat the problem of inefficient uptake and efficient efflux of antibiotics is to select a target that is sparse and therefore requires relatively little drug for effective inhibition. There are no more than 5-10 DNA polymerase III holoenzyme molecules per bacterial cell (Wu, Y. H., Franden, M. A., Hawker Jr, J. R., and McHenry, C. S. (1984) J Biol Chem 259, 12117-12122). In addition, the substrate for the holoenzyme, genomic DNA, is also sparse. Excessive or prolonged stalling of replication forks leads to template destruction by nucleases and cell death (Nakayama, K., Kusano, K., Irino, N., and Nakayama, H. (1994) J Mol Biol. 243, 611-620). Thus, very few molecules need to be inactivated to abolish the DNA replication capacity of a bacterial cell and antibacterial drugs that target the replication system may be especially well suited for the treatment of P. aeruginosa infections.

Yersinia pestis. Yersinia pestis is the causative agent of the historic ‘plague’ that killed approximately 17-28 million Europeans (ca. one-third of the total population) in the fourteenth century (Tuchman, B. W. (1978) A Distant Mirror: The Calamitous 14th Century, Random House, New York, Chapter 5; Raoult, D., Aboudharam, G., Crubezy, E., Larrouy, G., Ludes, B., and Drancourt, M. (2000) Proc Natl Acad Sci U S A 97, 12800-12803). Plague is a zoonotic infection; the disease is primarily one of rodent populations, not being dependent on man to maintain reservoirs (Keeling, M. J. and Gilligan, C. A. (2000) Nature 407, 903-906; McGovern, T. and Friedlander, A. (1997) Textbook of Military Medicine: Medical Aspects of Chemical and Biological Warfare. Borden Institute, Walter Reed Army Medical Center, Washington, D.C., Chapter 23). Fleas, upon ingesting a blood meal from an infected animal, support Y. pestis growth that eventually blocks the flea's foregut with a fibroid mass. When an infected flea attempts to feed again, it regurgitates clotted blood and bacteria into the victim's blood stream, and so passes the infection on to the next animal.

Man is an accidental host of Y. pestis infection. The problem is exacerbated when a particularly pathogenic strain of Y. pestis becomes epizootic, killing the natural rodent population and causing the fleas to pursue less preferred hosts. Upon infection of man, during the incubation phase, the Y. pestis bacilli spread to the lymph nodes, producing lymphadenitis and the characteristic swollen nodes of bubonic plague. Untreated infections progress to septicemia and spread to other organs, including spleen, liver, lungs, skin and mucous membranes. Primary pneumonic plague can result from direct inhalation of aerosols. This results in the most severe form of the disease and is more rapidly fatal since the inhaled droplets contain the phagocytosis-resistant form of bacilli that develop during growth at 37° C. in a mammalian host. Primary septicemic plague can occur from direct inoculation of bacilli into the bloodstream, bypassing initial multiplication in the lymph nodes.

Y. pestis is a rod-shaped, non-motile, non-sporulating, gram-negative, facultative anaerobe of the gamma division of proteobacteria. It is dependent on extra-chromosomal plasmids to encode essential pathogenesis genes. The bacterium can remain viable at near freezing temperatures for months or years. It also remains viable in dry sputum, flea feces and buried bodies, but is killed by exposure to sunlight for several hours. The wild-type bacteria are susceptible to standard antibacterials, especially streptomycin and tetracycline derivatives (McGovern & Friedlander, ibid.). Ofloxacin and ceftriaxone have been shown to be effective in animal models (Ibid.). Naturally occurring strains of Y. pestis have been isolated that are resistant to streptomycin, tetracycline, ampicillin, kanamycin and sulfonamides. The resistance element mapped to a transferable plasmid (Galimand, M., Guiyoule, A., Gerbaud, G., Rasoamanana, B., Chanteau, S., Carniel, E., and Courvalin, P. (1997) N Engl J Med 337, 677-680). A vaccine formulated from killed suspensions of virulent bacilli is available (McGovern & Friedlander, ibid). The normal vaccine dose schedule follows a six-month immunization course. An accelerated protocol taking 14 days has been developed, but no significant supporting data on the efficacy is available (Ibid.).

Y. pestis represents a significant threat. As a natural endemic threat, the World Health Organization has classified it as a re-emerging infectious disease. With the detection of drug-resistant strains and outbreaks in multiple parts of the world annually, it represents a serious natural threat (Boisier, P., Rasolomaharo, M., Ranaivoson, G., Rasoamanana, B., Rakoto, L., Andrianirina, Z., Andriamahefazafy, B., and Chanteau, S. (1997) Trop Med Int Health 2, 422-427; Barreto, A., Aragon, M., and Epstein, P. R. (1995) Lancet 345, 983-984). Used as a biological weapon and delivered and taken up as an aerosol, Y. pestis produces pulmonary syndromes characteristic of the endemic disease the bacteria produces in nature. (The infamous plague outbreak of the 14th century began as a result of the use of Y. pestis as a biological weapon. In 1346, during the siege of the Genoese northern Black Sea port of Caffa, invading Mongols catapulted dead solders that had succumbed to a deadly disease over the walls of the city in order to break its resistance. As the disease spread rapidly inside the city, the residents fled on merchant ships to Europe (Tuchman, ibid.). A form of Y. pestis, engineered to be constitutively in the phagocytosis-resistant form, drug-resistant and with a modified capsule to partially overcome immune surveillance presents a frightening possibility. Drug resistance mechanisms have now evolved for all clinically available antibacterials (see following sections) and it is also possible to engineer bacteria so that they become resistant to these drugs. A report appeared in the popular press, originating from a defector of the former Soviet Union, that claimed that the Soviet Union had a program for five years to develop drug-resistant strains of Y. pestis for purposes of biological warfare (McGovern & Friedlander, ibid.; Barry, J. (1993) Newsweek (February 1), 40-41). One possible method to circumvent this threat is to develop a novel class of antibacterial agent that operates against new targets for which a resistance mechanism has not yet evolved.

Accordingly, there remains a need for a chromosomal replication system to discover novel therapeutic agents that inhibit the central DNA replication apparatus of P. aeruginosa and Y. pestis.

### SUMMARY OF THE INVENTION

The present invention provide methods for screening compounds that modulate the activity of bacterial DNA replicases. In one embodiment, the method comprises contacting an isolated replicase with at least one test compound under conditions permissive for replicase activity; assessing the activity of the replicase in the presence of the test compound; and, comparing the activity of the replicase in the presence of the test compound with the activity of the replicase in the absence of the test compound, wherein a change in the activity of the replicase in the presence of the test compound is indicative of a compound that modulates the activity of the replicase, wherein said replicase comprises an isolated Y. pestis or P. aeruginosa DNA polymerase III subunit protein.

In another embodiment, the invention provides a method of identifying compounds which modulate the activity of a DNA polymerase III replicase, the method comprising forming a reaction mixture that includes a DNA template molecule, a DnaG primase, a DNA polymerase a subunit, a candidate compound, a mixture of NTPs and dNTPs, and optionally, a member of the group consisting of a α subunit, a τ complex, and both the β subunit and the τ complex, to form a replicase, subjecting the reaction mixture to conditions effective to achieve nucleic acid polymerization in the absence of the candidate compound, comparing the activity of the replicase in the presence of the test compound with the activity of the replicase in the absence of the test compound, wherein a change in the activity of the replicase in the presence of the test compound is indicative of a compound that modulates the activity of the replicase, wherein said replicase comprises a Y. pestis or P. aeruginosa DNA polymerase III subunit protein.

The present invention also include compound that modulates the activity of a bacterial DNA replicase identified by these methods.

Another embodiment of the present invention is a method for detecting functional activity of P. aeruginosa or Y. pestis DNA polymerase III protein subunits. A preferred method is the detection of activity comprising a) providing a test sample suspected of containing DNA polymerase III holoenzyme subunit protein; and b) comparing the activity of the test holoenzyme subunit in the sample with a quantitated DNA polymerase III holoenzyme subunit in a control to determine the relative activity of the test DNA polymerase III holoenzyme subunit in the sample. In one embodiment the activity is polymerase gap-filling activity for the detection of DNA polymerase III α subunit. In another embodiment, the activity is the stimulation of the processivity of the DNA polymerase for detection of the β subunit. In another embodiment the activity is binding to dnaA boxes for detection of DnaA. In another embodiment DnaX subunit is capable of stimulation of the processivity of the DNA polymerase in a reconstitution assay. In another embodiment δ subunit is capable of stimulation of the processivity of the DNA polymerase in a reconstitution assay. In another embodiment δ subunit is capable of stimulation of the processivity of the DNA polymerase in a reconstitution assay. Examples of such methods may be found in the Examples section.

The present invention also provides methods for screening antibacterial drug candidates that inhibit replicase activity of P. aeruginosa or Y. pestis DNA polymerase holoenzyme. This method comprises the steps of a) providing a test inhibitor suspected of inhibiting DNA polymerase III holoenzyme replication, b) detecting the DNA polymerase III replication reaction in test and control reaction, and c) comparing the test to the control, wherein the amount of replication correlates with the inhibitory effect of the test inhibitor.

The present invention relates to gene and amino acid sequences encoding DNA polymerase III holoenzyme subunits from P. aeruginosa, P. aeruginosa genes and nucleic acid molecules, including those that encode such proteins and to antibodies raised against such proteins. The present invention relates to gene and amino acid sequences encoding DNA polymerase III holoenzyme subunits from Y. pestis, Y. pestis genes and nucleic acid molecules, including those that encode such proteins and to antibodies raised against such proteins. The present invention also includes methods to obtain such proteins, nucleic acid molecules and antibodies.

One embodiment of the present invention includes an isolated P. aeruginosa or Y pestis DNA polymerase III protein, including DnaE, DnaN, DnaX, HolA, and HolB. A preferred P. aeruginosa or Y. pestis DNA polymerase III protein is capable of performing the function of that subunit in a functional assay. In one embodiment, DnaE is capable of extending primed DNA in a gap-filling polymerase assay. In another embodiment DnaN is capable of stimulation of the processitivity of the DNA polymerase in a processivity stimulation assay. In another embodiment, DnaX is capable of hydrolyzing ATP in a DNA-dependent manner. In another embodiment, HolA and HolB in the presence of DnaX are capable of loading DnaN onto primed DNA template. In another embodiment, DnaE, DnaN, DnaX, HolA and HolB are capable of assembling into a functional DNA replicase that can perform rapid and processive DNA synthesis on RNA- or DNA-primed long single-standed DNA templates. The present invention also relates to fusion proteins and mimetopes of P. aeruginosa or Y. pestis DNA polymerase III proteins as well as to isolated antibodies that selectively bind to P. aeruginosa or Y. pestis DNA polymerase III proteins or mimetopes thereof Also included are methods, including recombinant methods, to produce proteins, mimetopes and antibodies of the present invention.

Another embodiment of the present invention is an isolated P. aeruginosa or Y. pestis nucleic acid molecule that hybridizes under stringent hybridization conditions with P. aeruginosa or Y. pestis dnaE gene, P. aeruginosa or Y. pestis dnaN gene, P. aeruginosa or Y. pestis dnaQ gene, P. aeruginosa or Y. pestis holE gene, P. aeruginosa or Y. pestis dnaX gene, P. aeruginosa or Y. pestis holA gene, P. aeruginosa or Y. pestis holB gene, P. aeruginosa or Y. pestis holC gene, P. aeruginosa or Y. pestis holD gene, P. aeruginosa or Y. pestis ssb gene, P. aeruginosa or Y. pestis dnaG gene.

Y. pestis dnaE gene preferably includes nucleic acid sequence SEQ ID NO:1; Y. pestis dnaN gene preferably includes SEQ ID NO:18; Y. pestis dnaQ gene preferably includes SEQ ID NO:8. Y. pestis holE gene preferably includes nucleic acid sequence SEQ ID NO:13. Y. pestis dnaX gene preferably includes nucleic acid sequence SEQ ID NO:23. Y. pestis holA gene preferably includes nucleic acid sequence SEQ ID NO:33. Y. pestis holB gene preferably includes nucleic acid sequence SEQ ID NO:38. Y. pestis holC gene preferably includes nucleic acid sequence SEQ ID NO:43. Y. pestis holD gene preferably includes nucleic acid sequence SEQ ID NO:48. Y. pestis ssb gene preferably includes nucleic acid sequence SEQ ID NO:53. Y. pestis dnaG gene preferably includes nucleic acid sequence SEQ ID NO:58.

P. aeruginosa dnaE gene preferably includes nucleic acid sequence SEQ ID NO:65. P. aeruginosa dnaN gene preferably includes SEQ ID NO:112. P. aeruginosa dnaQ gene preferably includes SEQ ID NO:75. P. aeruginosa dnaX gene preferably includes nucleic acid sequence SEQ ID NO:80. P. aeruginosa holA gene preferably includes nucleic acid sequence SEQ ID NO:85. P. aeruginosa holB gene preferably includes nucleic acid sequence SEQ ID NO:90. P. aeruginosa holC gene preferably includes nucleic acid sequence SEQ ID NO:95. P. aeruginosa ssb gene preferably includes nucleic acid sequence SEQ ID NO:107. P. aeruginosa dnaG gene preferably includes nucleic acid sequence SEQ ID NO:102.

DNA Polymerase III nucleic acid molecule of the present invention can include a regulatory region of a P. aeruginosa or Y. pestis DNA Polymerase III gene and/or can encode a P. aeruginosa or Y. pestis DNA Polymerase III protein.

The present invention also relates to recombinant molecules and recombinant cells that include at least a portion of P. aeruginosa or Y. pestis DNA polymerase III nucleic acid molecule of the present invention. Also included are methods to produce such nucleic acid molecules, recombinant molecules and recombinant cells.

The present invention also provides an antibody, wherein the antibody is capable of specifically binding to at least one antigenic determinant on the protein encoded by an amino acid sequence of the present invention. The antibodies of the present invention may be prepared using various immunogens. In one embodiment, the immunogen is DNA polymerase III holoenzyme or holoenzyme subunit peptide, to generate antibodies that recognize DNA polymerase III holoenzyme or holoenzyme subunit(s). Such antibodies include, but are not limited to polyclonal, monoclonal, chimeric, single chain, Fab fragments, and an Fab expression library. Various procedures known in the art may be used for the production of polyclonal or monoclonal antibodies to DNA polymerase III holoenzyme or holoenzyme subunits.

The present invention also provides methods for detecting DNA polymerase III comprising: providing in any order, a sample suspected of containing DNA polymerase III, and antibody capable of specifically binding to at least a portion of the DNA polymerase III; mixing the sample and the antibody under conditions wherein the antibody can bind to the DNA polymerase III; and detecting the binding. In preferred embodiments of the methods, the sample comprises a P. aeruginosa or Y. pestis.

The present invention also provides methods for producing anti-DNA polymerase III holoenzyme and anti-DNA polymerase III holoenzyme subunit antibodies, exposing an animal having immunocompetent cells to an immunogen comprising at least an antigentic portion of DNA polymerase III holoenzyme or DNA polymerase III holoenzyme subunit. In one embodiment, the method further comprises the step of harvesting the antibodies. In an alternative embodiment, the method comprises the step of fusing the immunocompetent cells with an immortal cell line under conditions such that a hybridoma is produced.

The present invention also provides methods for detection of nucleic acid molecules encoding at least a portion of DNA polymerase III holoenzyme, or DNA polymerase III holoenzyme subunit in a biological sample comprising the steps of: a) hybridizing at least a portion of a nucleic acid molecule of the present invention to nucleic acid material of a biological sample, thereby forming a hybridization complex, and b) detecting the hybridization complex, wherein the presence of the complex correlates with the presence of a polynucleotide encoding at least a portion of DNA polymerase III holoenzyme or DNA polymerase III holoenzyme subunit in the biological sample. In alternative preferred embodiment of the methods, the nucleic acid material of the biological sample is amplified by the polymerase chain reaction.

The present invention also provides methods for detecting DNA polymerase III holoenzyme or holoenzyme subunit expression, including expression of abnormal or mutated DNA polymerase III holoenzyme or holoenzyme subunint proteins or gene sequences comprising the steps of a) providing a test sample suspected of containing DNA polymerase III holoenzyme or DNA polymerase III holoenzyme subunit protein, as appropriate; and b) comparing test DNA polymerase III holoenzyme or holoenzyme subunit with quantitated DNA polymerase III holoenzyme or holoenzyme subunit in a control to determine the relative concentration of the test DNA polymerase III holoenzyme or holoenzyme subunit in the sample. In addition, the methods may be conducted using any suitable means to determine the relative concentration of DNA polymerase holoenzyme or holoenzyme subunit in the test and control samples.

This invention provides methods for the assembly of a complete P. aeruginosa chromosomal DNA replication elongation system. This invention also provides methods for the expression and purification of the anticipated five centrally important components of the P. aeruginosa DNA polymerase III holoenzyme. This invention also provides methods for the expression and purification of the nine components of the P. aeruginosa DNA polymerase III holoenzyme. This invention further provides a P. aeruginosa minimal processive replicase. This invention also provides methods for the use of the fully reconstituted P. aeruginosa replication system in a high-throughput screening format to identify compounds that are active as inhibitors of Pseudomonas DNA replication.

This invention provides methods for the assembly of a complete Y. pestis chromosomal DNA replication elongation system. This invention also provides methods for the expression and purification of the anticipated five centrally important components of the Y. pestis DNA polymerase III holoenzyme. This invention also provides methods for the expression and purification of nine components of the Y. pestis DNA polymerase III holoenzyme. This invention further provides a Y. pestis minimal processive replicase. This invention also provides methods for the use of the fully reconstituted Y. pestis replication system in a high-throughput screening format to identify compounds that are active as modulators of Yersinia DNA replication.

The invention also provides methods for the molecular cloning of P. aeruginosa and Y. pestis dnaE, the gene encoding the replicative polymerase, the expression of DnaE in E. coli, its purification, and characterization of its intrinsic gap-filling activity.

The invention also provides method for the molecular cloning of P. aeruginosa and Y. pestis dnaN, the gene encoding the sliding clamp processivity factor for the bacterial replicase, the expression of DnaN in E. coli, its purification, and characterization of its contribution to the processivity of the corresponding DnaE.

The invention also provides method for the molecular cloning of P. aeruginosa and Y. pestis dnaX, the gene encoding the DNA-dependent ATPase clamp loader, P. aeruginosa and Y. pestis holA and holB, the genes encoding essential accessory subunits of the DnaX complex, the expression of DnaX, HolA and HolB in E. coli and their purification.

The invention also provides method for the molecular cloning of P. aeruginosa and Y. pestis holC and holD, ssb, and dnaG genes, the expression of HolC, HolD, SSB and DnaG proteins in E. coli and their purification.

The invention also provides method for the molecular cloning of Y. pestis holC and holD, ssb, and dnaG genes, the expression of HolC, HolD, SSB and DnaG proteins in E. coli and their purification.

The invention also provides methods for the use the above proteins to reconstitute a minimal DNA polymerase III holoenzyme from P. aeruginosa and Y. pestis capable of rapid and processive DNA synthesis on primed single-stranded templates and to adapt the reconstituted enzyme to the Multiplicative Target Screening™ assay.

### BRIEF DESCRIPTION OF THE FIGURES

FIG. 1 shows the stages in replication of M13Gori DNA. The standard assay used for the DNA polymerase III holoenzyme is the conversion of single-stranded M13Gori DNA to the duplex replicative form. In routine laboratory assays, incorporation of a radioactive nucleotide into acid precipitable DNA is monitored. In the high-throughput assay used here, the assay is quenched with EDTA containing Picogreen™. Picogreen fluoresces when bound to double-stranded DNA, permitting quantitation of DNA synthesized. Priming Step: The G4 origin cloned into M13 DNA is specifically recognized by the E. coli DnaG primase in the presence of SSB, permitting generation of a short RNA primer. Initiation Complex Formation Step: The holoenzyme recognizes the primer and forms an ATP-dependent initiation complex. The DnaX ATPase serves to transfer the β processivity factor onto DNA in this stage of the reaction. Elongation Step: Upon addition of all four dNTPs (dATP, dGTP, dCTP and dTTP) to initiation complex, holoenzyme replicates the entire circle without dissociation.

FIG. 2 shows structural features of the DNA polymerase III holoenzyme.

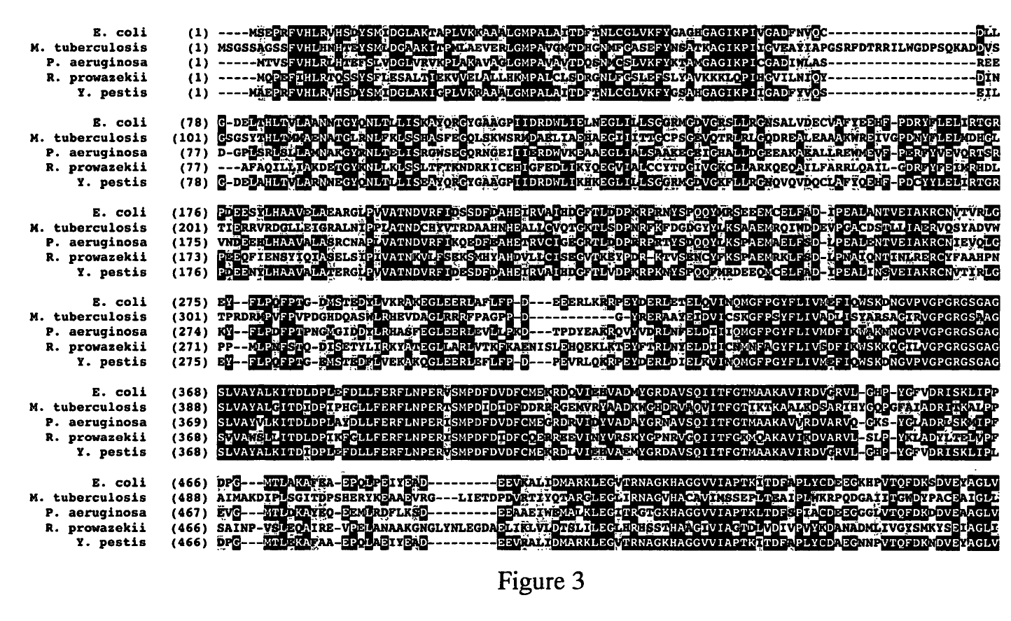

FIG. 3 shows alignment of amino acid sequences for DnaE from Escherichia coli (SEQ ID NO:139), Mycobacterium tuberculosis (SEQ ID NO:140), P. aeruginosa (SEQ ID NO:72), Rickettsia prowazekii (SEQ ID NO:141) and Y. pestis (SEQ ID NO:3). Identical residues are highlighted in black and similar residues are highlighted in gray.

FIG. 4 shows algnment of amino acid sequences for DnaN from Escherichia coli (SEQ ID NO:142), Mycobacterium tuberculosis (SEQ ID NO:143), P. aeruginosa (SEQ ID NO:114), Rickettsia prowazekii (SEQ ID NO:144) and Y. pestis (SEQ ID NO:20). Identical residues are highlighted in black and similar residues are highlighted in gray.

FIG. 5 shows alignment of amino acid sequences for DnaX from Escherichia coli (SEQ ID NO:145), Mycobacterium tuberculosis (SEQ ID NO:146), P. aeruginosa (SEQ ID NO:82), Rickettsia prowazekii (SEQ ID NO:147) and Y. pestis (SEQ ID NO:25). Identical residues are highlighted in black and similar residues are highlighted in gray.

FIG. 6 shows alignment of amino acid sequences for HolA (upper panel) and HolB (lower panel) from Escherichia coli (SEQ ID NO:148, SEQ ID NO:151), Mycobacterium tuberculosis (SEQ ID NO:149, SEQ ID NO:152), P. aeruginosa (SEQ ID NO:87, SEQ ID NO:92), Rickettsia prowazekii (SEQ ID NO:150, SEQ ID NO:153) and Y. pestis (SEQ ID NO:35, SEQ ID NO:40). Identical residues are highlighted in black and similar residues are highlighted in gray.

FIG. 7 shows alignment of amino acid sequences for HolC (chi), HolD (psi), DnaQ (epsilon) and HolE (theta) from Escherichia coli (SEQ ID NO:154, SEQ ID NO:155, SEQ ID NO:156) and Y. pestis (SEQ ID NO:45, SEQ ID NO:50, SEQ ID NO:10, SEQ ID NO:15). Identical residues are highlighted in black and similar residues are highlighted in gray.

FIG. 8A-8C show alignment of amino acid sequences for SSB, DnaB and DnaG proteins from Escherichia coli (SEQ ID NO:157, SEQ ID NO:158, SEQ ID NO:159) and Y. pestis (SEQ ID NO:55, SEQ ID NO:60). Identical residues are highlighted in black and similar residues are highlighted in gray.

FIG. 9 shows the region of plasmid pA1-CB-NcoI encompassing the polyclonal region (SEQ ID NO:121 and SEQ ID NO:122).

FIG. 10 shows the region of plasmid pA1-CB-NdeI encompassing the polyclonal region (SEQ ID NO:123 and SEQ ID NO:124).

FIG. 11 shows a schematic depiction of pA1-YP-dneE5′.

FIG. 12 shows a schematic depiction of pA1-YP-dnaE.

FIG. 13 shows a schematic depiction of pA1-YP-dnaQ.

FIG. 14 shows a schematic depiction of pA1-YP-holE.

FIG. 15 shows a schematic depiction of pA1-YP-QE.

FIG. 16 shows a schematic depiction of pA1-YP-core.

FIG. 17 shows expression of Y. pestis pol III core subunits as a function of growth time following IPTG induction.

FIG. 18 shows the determination of optional ammonium sulfate precipitation conditions for obtaining maximum amounts of Y. pestis core.

FIG. 19 shows a summary of results for the determination of optimal ammonium sulfate precipitation conditions for obtaining the maximum amounts of activity.

FIG. 20 shows activity vs. component mixture in reconstution assays to determine if Y. pestis is active when combined with E. coli τ-complex and E. coli β.

FIG. 21 shows a summary of SDS-polyacrylamide gel analysis of each step in the purification of Y. pestis core.

FIG. 22 shows a schematic depiction of pA1-YP-dnaN.

FIG. 23 shows a gel of expression of Y. pestis β subunit as a function of growth time following IPTG induction FIG. 24 shows activity vs. percent ammonium sulfate results for determination of optimal ammonium sulfate precipitation conditions for Y. pestis β subunit.

FIG. 25 shows a a summary gel of Y. pestis β purification.

FIG. 26 shows DNA synthesis vs. Y. pestis β in a titration of Y. pestis β subunit in the assay mixture containing E. coli pol III core and τ complex.

FIG. 27 shows activity vs. per cent ammonium sulfate in an assay mixture containing E. coli pol III core and τ complex, and indicates that Y. pestis β is most efficiently precipitated out of solution at high concentrations of ammonium sulfate

FIG. 28 shows a schematic depiction of pA1-YP-DnaX.

FIG. 29 shows a schematic depiction of pA1-YP-holA.

FIG. 30 shows a schematic depiction of pA1-YP-holB

FIG. 31 shows the region of plasmid pA1-CB-NcoI encompassing the polyclonal region.

FIG. 32 shows a schematic depiction of pA1-YP-holC.

FIG. 33 shows a schematic depiction of pA1-YP-holD.

FIG. 34 shows a schematic depiction of pA1-YP-holCD.

FIG. 35 shows a schematic depiction of pA1-YP-holBA.

FIG. 36 shows a schematic depiction of pA1-YP-holBAX.

FIG. 37 shows a schematic depiction of pA1-YP-CLcomplex.

FIG. 38 shows expression of Y. pestis τ complex as a function of grouth tme following IPTG induction.

FIG. 39 shows protein concentration vs. ammonium sulfate concentration for Y. pestix τ complex

FIG. 40 shows total amount of activity in a reconstitution assay for each of the ammonium sulfate cuts of Y. pestis τ complex.

FIG. 41 shows an SDS polyacrylamide gel analysis of purification of Y. pestis τ complex.

FIG. 42A-C shows the results of experiments to determine the concentration determined to have maximum activity for β₂, core, and τ complex.

FIG. 43 shows a schematic depiction of pA1-YP-ssb.

FIG. 44 shows growth optimization analyzed by SDS-polyacrylamide gel electrophoresis for E. coli containing pA1-YP-ssb.

FIG. 45 the results of optimal ammonium sulfate precipitation conditions for obtaining maximum amounts of Y. pestis SSB.

FIG. 46 shows the results of SDS polyacrylamide gel analysis of Fr IIs from ammonium sulfate optimization experiments of Y. pestis SSB.

FIG. 47 shows a summary gel of the pooled protein for each purification step for ssb.

FIG. 48 shows the growth optimization of Y. pestis DnaG.

FIG. 49 shows SDS-polyacrylamide gel analysis of the Fr II samples resulting from proteins precipitated in different concentrations of ammonium sulfate.

FIG. 50 shows a schematic depiction of pA1-YP-dnaG.

FIG. 51 shows activity analysis and ammonium sulfate optimization of Y. pestis DnaG.

FIG. 52 shows a comparison Y. pestis DnaG FrII preparations with that of FrII containing only E. coli endogenous proteins to determine the contribution of endogenous E. coli DnaG in reconstitution assays.

FIG. 53 shows a summary gel of the pooled protein for each purification step of DnaG.

FIG. 54 shows a schematic depiction of pA1-PA-dnaE#1-5′.

FIG. 55 shows a schematic depiction of pA1-PA-dnaE#1

FIG. 56 shows a schematic depiction of pA1-PA-dneE#2-5′.

FIG. 57 shows a schematic depiction of pA1-PA-dnaE#2.

FIG. 58 shows a schematic depiction of pA1-PA-dnaQ.

FIG. 59 shows a schematic deptiction of PA1-PA-core #1.

FIG. 60 shows a schematic deptiction of PA1-PA-core #2.

FIG. 61 shows a schematic depiction of pA1-PA-dnaN.

FIG. 62 shows a schematic depiction of pA1-PA-dnaX

FIG. 63 shows a schematic depiction of pA1-PA-holA.

FIG. 64 shows a schematic depiction of pA1-PA-holB.

FIG. 65 shows the region of plasmid pA1-NB-KpnI encompassing the polyclonal region (SEQ ID NO:137 and SEQ ID NO:138).

FIG. 66 shows the region of pA1-NB-KpnI after the holC PCR product was inserted (SEQ ID NO:131, SEQ ID NO:132 and SEQ ID NO:133).

FIG. 67 shows a schematic depiction of pA1-NB-PAholC.

FIG. 68 shows a schematic depiction of PUCP19-Re.

FIG. 69 shows the region of plasmid pUCP19 encompassing the polyclonal region (SEQ ID NO:125 and SEQ ID NO:126).

FIG. 70 shows a schematic depiction of pUCP19-NB-PAholC.

FIG. 71 shows the region of plasmid pA1-CB-NsiI encompassing the polyclonal region (SEQ ID NO:127 and SEQ ID NO:128).

FIG. 72 shows a schematic depiction of pA1-CB-PAholC.

FIG. 73 shows a schematic depiction of pUCP19-CB-PAholC.

FIG. 74 shows a schematic depiction of pA1-PA-holBA.

FIG. 75 shows a schematic depiction of pA1-PA-dnaG.

FIG. 76 shows a schematic depiction of pA1-PA-ssb.

FIG. 77 shows a schematic depiction of pA1-PA-BAX.

FIG. 78 shows activity of DnaG in a modified reconstitution assay.

### DETAILED DESCRIPTION OF THE PREFERRED EMBODIMENTS

The present invention relates to gene and amino acid sequences encoding DNA polymerase III holoenzyme subunits and structural genes from P. aeruginosa and Y. pestis. As used herein, the term “gene” refers to a nucleic acid (e.g., DNA) sequence that comprises coding sequences necessary for the production of a polypeptide or precursor (e.g., DNA polymerase III holoenzyme or holoenzyme subunit, as appropriate). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The term “gene” encompasses both cDNA and genomic forms of a gene. A genomic form or clone of a gene contains the coding region interrupted with non-coding sequences termed “intervening regions” or “intervening sequences.” The mRNA functions during translation to specify the sequence or order of amino acids in a nascent polypeptide. As used herein, the term “DNA polymerase III holoenzyme” refers to the entire DNA polymerase III entity (i.e., all of the polymerase subunits, as well as the other associated accessory proteins required for processive replication of a chromosome or genome), while “DNA polymerase III” is just the polymerase core [α, ε, θ subunits in E. coli]). “DNA polymerase III holoenzyme subunit” is used in reference to any of the subunit entities that comprise the DNA polymerase III holoenzyme. Thus, the term “DNA polymerase III holoenzyme” encompasses “DNA polymerase III” and “DNA polymerase III subunits.” Subunits include, but may not be limited to DnaE, DnaN (the beta (β) processivity factor), DnaX, HolA, HolB, SSB, DnaG and DnaB proteins. Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited proteins.

Amino Acid Sequences and Proteins

One embodiment of the present invention is an isolated DNA polymerase III holoenzyme subunit protein. According to the present invention, an isolated, or biologically pure, protein, is a protein that has been removed from its natural environment. As such, “isolated” and “biologically pure” do not necessarily reflect the extent to which the protein has been purified. An isolated P. aeruginosa or Y. pestis DNA polymerase III holoenzyme subunit protein of the present invention can be obtained from its natural source, can be produced using recombinant DNA technology or can be produced by chemical synthesis. As used herein, an isolated P. aeruginosa or Y. pestis DNA polymerase III holoenzyme subunit protein can be a full-length protein or any homologue of such a protein. A preferred DNA polymerase III holoenzyme subunit protein of the present invention is P. aeruginosa or Y. pestis DNA polymerase III holoenzyme subunit, including the DNA polymerase subunit DnaE protein, DnaQ protein, DnaN protein, DnaX protein, HolA protein, HolB protein, HolC protein, HolD, protein, SSB protein, DnaG protein, or a homolog of any of these subunits (including, but not limited to the encoded proteins, full-length proteins, processed proteins, fusion proteins and multivalent proteins thereof) as well as proteins that are truncated homologs of proteins that include at least portions of the aforementioned proteins. Another embodiment of the present invention includes an isolated P. aeruginosa or Y. pestis DNA polymerase III subunit protein, including the DNA polymerase DnaE protein, DnaN protein, DnaX protein, HolA protein, and HolB protein. In one embodiment, a preferred DNA polymerase III DnaE subunit protein has a molecular weight of about 110-135 kDa as determined by Tris-glycine SDS PAGE. In another embodiment, DNA polymerase III DnaN subunit has a molecular weight of about 40-41 kDa as determined by Tris-glycine SDS PAGE. In another embodiment, DnaX has a molecular weight of about 71-73 kDa (for the full-length product of the gene) as determined by Tris-glycine SDS PAGE. In another embodiment, HolA protein has a molecular weight of about 37-40 kDa as determined by Tris-glycine SDS PAGE. In another embodiment, HolB protein has a molecular weight of about 35-38 kDa as determined by Tris-glycine SDS PAGE. In another embodiment, HolC protein has a molecular weight of about 15-17 kDa as determined by Tris-glycine SDS PAGE. In another embodiment, HolD protein has a molecular weight of about 15-17 kDa as determined by Tris-glycine SDS PAGE. Particularly preferred Y. pestis DNA polymerase III proteins include DnaE (α), represented by amino acid sequence SEQ ID NO:3, DnaQ (ε), represented by amino acid sequence SEQ ID NO:10, HolE (θ), represented by amino acid sequence SEQ ID NO:15, DnaN (β), represented by amino acid sequence SEQ ID NO:20, DnaX (γ/τ), represented by amino acid sequence SEQ ID NO:25, HolA (δ), represented by amino acid sequence SEQ ID NO:35, HolB (δ′), represented by amino acid sequence SEQ ID NO:40, HolC (X), represented by amino acid sequence SEQ ID NO:45, HolD (ψ), represented by amino acid sequence SEQ ID NO:50, ssb, represented by amino acid sequence SEQ ID NO:55, and/or amino acid sequence DnaG (primase), represented by SEQ ID NO:60. Particularly preferred P. aeruginosa DNA polymerase III proteins include DnaE (α), represented by amino acid sequence SEQ ID NO:67, DnaE (α), represented by amino acid sequence SEQ ID NO:72, DnaQ (ε), represented by amino acid sequence SEQ ID NO:77, DnaX (γ/τ), represented by amino acid sequence SEQ ID NO:82, HolA (δ), represented by amino acid sequence SEQ ID NO:87, HolB (δ′), represented by amino acid sequence SEQ ID NO:92, HolC (χ), represented by amino acid sequence SEQ ID NO:97, SSB, represented by amino acid sequence SEQ ID NO:104, DnaG (primase), represented by amino acid sequence SEQ ID NO:109, and/or DnaN (β), represented by amino acid sequence SEQ ID NO: 114, as well as proteins that are encoded by nucleic acid molecules that are allelic variants of the nucleic acid molecules that encode proteins having any of those SEQ ID NO's. Examples of methods to produce such proteins are disclosed herein.

A preferred P. aeruginosa or Y. pestis DNA polymerase III protein subunit is capable of performing the function of that subunit in a functional assay. In one embodiment, DnaE is capable of extending primed DNA in a gap-filling polymerase assay. In another embodiment DnaN is capable of stimulation of the processitivity of the DNA polymerase in the presence of DnaN in a processivity stimulation assay. In another embodiment, DnaX is capable of hydrolyzing ATP in a DNA-dependent manner. In another embodiment, HolA and HolB in the presence of DnaX are capable of loading DnaN onto primed DNA template. In another embodiment, DnaE, DnaN, DnaX, HolA and HolB are capable of assembling into a functional DNA replicase that can perform rapid and processive DNA synthesis on RNA- or DNA-primed long single-standed DNA templates. Examples of such assays are detailed in the Examples section. The ability of such protein subunits to function in an activity detection assay suggests the utility of such proteins and mimetopes in an assay to screen for antibacterial drug candidates that inhibit P. aeruginosa or Y. pestis replicase. As used herein, “replicase” means an enzyme that duplicates a DNA polynucleotide sequence.

The phrase “capable of performing the function of that subunit in a functional assay” means that the protein has at least about 10% of the activity of the natural protein subunit in the functional assay. In other preferred embodiments, has at least about 20% of the activity of the natural protein subunit in the functional assay. In other preferred embodiments, has at least about 30% of the activity of the natural protein subunit in the functional assay. In other preferred embodiments, has at least about 40% of the activity of the natural protein subunit in the functional assay. In other preferred embodiments, has at least about 50% of the activity of the natural protein subunit in the functional assay. In other preferred embodiments, the protein has at least about 60% of the activity of the natural protein subunit in the functional assay. In more preferred embodiments, the protein has at least about 70% of the activity of the natural protein subunit in the functional assay. In more preferred embodiments, the protein has at least about 80% of the activity of the natural protein subunit in the functional assay. In more preferred embodiments, the protein has at least about 90% of the activity of the natural protein subunit in the functional assay.

As used herein, an isolated protein of the present invention can be a full-length protein or any homolog of such a protein, such as a protein in which amino acids have been deleted, inserted, inverted, substituted and/or derivatized (e.g., by glycosylation, phosphorylation, acetylation, myristoylation, prenylation, palmitoylation, amidation and/or addition of glycerophosphatidyl inositol) such that the homolog comprises a protein having an amino acid sequence that is sufficiently similar to a natural P. aeruginosa or Y. pestis DNA polymerase protein that a nucleic acid sequence encoding the homolog is capable of hybridizing under stringent conditions to (i.e., with) the complement of a nucleic acid sequence encoding the corresponding natural P. aeruginosa or Y. pestis DNA polymerase amino acid sequence. As used herein, stringent hybridization conditions refer to standard hybridization conditions under which nucleic acid molecules, including oligonucleotides, are used to identify similar nucleic acid molecules. Such standard conditions are disclosed, for example, in Sambrook et al., Molecular Cloning: A Laboratory Manual, Cold Spring Harbor Labs Press, 1989; Sambrook et al., ibid., is incorporated by reference herein in its entirety. Stringent hybridization conditions typically permit isolation of nucleic acid molecules having at least about 70% nucleic acid sequence identity with the nucleic acid molecule being used to probe in the hybridization reaction. Formulae to calculate the appropriate hybridization and wash conditions to achieve hybridization permitting 30% or less mismatch of nucleotides are disclosed, for example, in Meinkoth et al., 1984, Anal. Biochem. 138, 267-284; Meinkoth et al., ibid., is incorporated by reference herein in its entirety. In preferred embodiments, hybridization conditions will permit isolation of nucleic acid molecules having at least about 80% nucleic acid sequence identity with the nucleic acid molecule being used to probe. In more preferred embodiments, hybridization conditions will permit isolation of nucleic acid molecules having at least about 90% nucleic acid sequence identity with the nucleic acid molecule being used to probe. In more preferred embodiments, hybridization conditions will permit isolation of nucleic acid molecules having at least about 95% nucleic acid sequence identity with the nucleic acid molecule being used to probe.

The minimal size of a protein homolog of the present invention is a size sufficient to be encoded by a nucleic acid molecule capable of forming a stable hybrid with the complementary sequence of a nucleic acid molecule encoding the corresponding natural protein. As such, the size of the nucleic acid molecule encoding such a protein homolog is dependent on nucleic acid composition and percent homology between the nucleic acid molecule and complementary sequence as well as upon hybridization conditions per se (e.g., temperature, salt concentration, and formamide concentration). The minimal size of such nucleic acid molecules is typically at least about 12 to about 15 nucleotides in length if the nucleic acid molecules are GC-rich and at least about 15 to about 17 bases in length if they are AT-rich. As such, the minimal size of a nucleic acid molecule used to encode a protease protein homolog of the present invention is from about 12 to about 18 nucleotides in length. There is no limit on the maximal size of such a nucleic acid molecule in that the nucleic acid molecule can include a portion of a gene, an entire gene, or multiple genes, or portions thereof. Similarly, the minimal size of a polymerase protein homolog of the present invention is from about 4 to about 6 amino acids in length, with preferred sizes depending on whether a full-length, multivalent (i.e., fusion protein having more than one domain each of which has a function), or functional portions of such proteins are desired. Polymerase protein homologs of the present invention preferably have activity corresponding to the natural subunit.

A protein homolog of the present invention can be the result of allelic variation of a natural gene encoding P. aeruginosa or Y. pestis DNA polymerase III holoenzyme subunit. A natural gene refers to the form of the gene found most often in nature. DNA polymerase holoenzyme III subunit homologs can be produced using techniques known in the art including, but not limited to, direct modifications to a gene encoding a protein using, for example, classic or recombinant DNA techniques to effect random or targeted mutagenesis. Isolated DNA polymerase III subunit proteins of the present invention, including homologs, can be identified in a straight-forward manner by the proteins ability to perform the subunit's specified function. Examples of such techniques are delineated in the Examples section.

The present invention also includes mimetopes of P. aeruginosa or Y. pestis DNA polymerase holoenzyme III subunit proteins. In accordance with the present invention, a mimetope refers to any compound that is able to mimic the ability of an isolated P. aeruginosa or Y. pestis DNA polymerase holoenzyme III subunit protein of the present invention to perform the function of that subunit in a functional assay. A mimetope can be a peptide that has been modified to decrease its susceptibility to degradation but that still retains functional ability. Other examples of mimetopes include, but are not limited to, anti-idiotypic antibodies or fragments thereof, that include at least one binding site that mimics one or more epitopes of an isolated protein of the present invention; non-proteinaceous immunogenic portions of an isolated protein (e.g., carbohydrate structures); and synthetic or natural organic molecules, including nucleic acids, that have a structure similar to at least one epitope of an isolated protein of the present invention. Such mimetopes can be designed using computer-generated structures of proteins of the present invention. Mimetopes can also be obtained by generating random samples of molecules, such as oligonucleotides, peptides or other organic molecules, and screening such samples by affinity chromatography techniques using the corresponding binding partner.

One embodiment of the present invention is a fusion protein that includes a P. aeruginosa or Y. pestis DNA polymerase holoenzyme III subunit protein-containing domain attached to a fusion segment. As used herein, the term “fusion protein” refers to a chimeric protein containing the protein of interest (i.e., DNA polymerase III holoenzyme or holoenzyme subunit and fragments thereof) joined to an exogenous protein fragment (the fusion partner which consists of a non-DNA polymerase III holoenzyme or holoenzyme subunit protein). The fusion partner may enhance solubility of the DNA polymerase III holoenzyme or holoenzyme subunit protein as expressed in a host cell, may provide an affinity tag to allow purification of the recombinant fusion protein from the host cell or culture supernatant, or both. If desired, the fusion protein may be removed from the protein of interest (i.e., DNA polymerase III holoenzyme, holoenzyme subunit protein, or fragments thereof) by a variety of enzymatic or chemical means known to the art. Inclusion of a fusion segment as part of a P. aeruginosa or Y. pestis DNA polymerase holoenzyme III subunit of the present invention can enhance the protein's stability during production, storage and/or use. Depending on the segment's characteristics, a fusion segment can also act as an immunopotentiator to enhance the immune response mounted by an animal immunized with P. aeruginosa or Y. pestis DNA polymerase holoenzyme III subunit protein containing such a fusion segment. Furthermore, a fusion segment can function as a tool to simplify purification of P. aeruginosa or Y. pestis DNA polymerase holoenzyme III subunit protein, such as to enable purification of the resultant fusion protein using affinity chromatography. A suitable fusion segment can be a domain of any size that has the desired function (e.g., imparts increased stability, imparts increased immunogenicity to a protein, and/or simplifies purification of a protein). It is within the scope of the present invention to use one or more fusion segments. Fusion segments can be joined to amino and/or carboxyl termini of the P. aeruginosa or Y. pestis DNA polymerase holoenzyme III subunit-containing domain of the protein. Linkages between fusion segments and P. aeruginosa or Y. pestis DNA polymerase holoenzyme III subunit-containing domains of fusion proteins can be susceptible to cleavage in order to enable straight-forward recovery of the P. aeruginosa or Y. pestis DNA polymerase holoenzyme III subunit-containing domains of such proteins. Fusion proteins are preferably produced by culturing a recombinant cell transformed with a fusion nucleic acid molecule that encodes a protein including the fusion segment attached to either the carboxyl and/or amino terminal end of a P. aeruginosa or Y. pestis DNA polymerase holoenzyme III subunit-containing domain.

Preferred fusion segments for use in the present invention include a glutathione binding domain, such as glutathione-S-transferase (GST) or a portion thereof capable of binding to glutathione; a metal binding domain, such as a poly-histidine segment capable of binding to a divalent metal ion; an immunoglobulin binding domain, such as Protein A, Protein G, T cell, B cell, F_{c} receptor or complement protein antibody-binding domains; a sugar binding domain such as a maltose binding domain from a maltose binding protein; and/or a “tag” domain (e.g., at least a portion of β-galactosidase, a strep tag peptide, other domains that can be purified using compounds that bind to the domain, such as monoclonal antibodies). More preferred fusion segments include metal binding domains, such as a poly-histidine segment; a maltose binding domain; and a hexahistidine/biotin binding peptide.

Nucleic Acid Molecules

Another embodiment of the present invention is an isolated nucleic acid molecule that hybridizes under stringent hybridization conditions with at least one of the P. aeruginosa or Y. pestis DNA polymerase III holoenzyme subunit genes of the present invention. A preferred P. aeruginosa gene is dnaE, and includes nucleic acid sequence SEQ ID NO:65 and SEQ ID NO:70, which encodes a dnaE subunit protein including SEQ ID NO:67 and SEQ ID NO:72. Another preferred P. aeruginosa gene is dnaQ, and includes nucleic acid sequence SEQ ID NO:75, which encodes a dnaQ protein including SEQ ID NO:77. Another preferred P. aeruginosa gene is dnaN, and includes nucleic acid sequence SEQ ID NO:112, which encodes a DNA polymerase III β-subunit protein including SEQ ID NO:114. Another preferred P. aeruginosa gene is dnaX, and includes nucleic acid sequence SEQ ID NO:80, which encodes a dnaX protein including SEQ ID NO:82. Another preferred P. aeruginosa gene is holA, and includes nucleic acid sequence SEQ ID NO:85, which encodes a δ subunit protein including SEQ ID NO:87. Another preferred P. aeruginosa gene is holB, and includes nucleic acid sequence SEQ ID NO:90, which encodes a δ′ subunit protein including SEQ ID NO:92. Another preferred P. aeruginosa gene is holC, and includes nucleic acid sequence SEQ ID NO:95, which encodes a χ subunit protein including SEQ ID NO:97. Another preferred P. aeruginosa gene is dnaG, and includes nucleic acid sequence SEQ ID NO:102, which encodes a dnaG subunit protein including SEQ ID NO:104. Another preferred P. aeruginosa gene is ssb, and includes nucleic acid sequence SEQ ID NO:107, which encodes a ssb subunit protein including SEQ ID NO:109.

A preferred Y. pestis gene is dnaE, and includes nucleic acid sequence SEQ ID NO:1, which encodes a dnaE subunit protein including SEQ ID NO:3. Another preferred Y. pestis gene is dnaQ, and includes nucleic acid sequence SEQ ID NO:8, which encodes a DNA polymerase III ε-subunit protein including SEQ ID NO:10. Another preferred Y. pestis gene is holE, and includes nucleic acid sequence SEQ ID NO:13, which encodes a DNA polymerase III θ-subunit protein including SEQ ID NO:15. Another preferred Y. pestis gene is dnaN, and includes nucleic acid sequence SEQ ID NO:18, which encodes a DNA polymerase III β-subunit protein including SEQ ID NO:20. Another preferred Y. pestis gene is dnaX, and includes nucleic acid sequence SEQ ID NO:23, which encodes a dnaX protein including SEQ ID NO:25. Another preferred Y. pestis gene is holA, and includes nucleic acid sequence SEQ ID NO:33, which encodes a δ subunit protein including SEQ ID NO:35. Another preferred Y. pestis gene is holB, and includes nucleic acid sequence SEQ ID NO:38, which encodes a δ′ subunit protein including SEQ ID NO:40. Another preferred Y. pestis gene is holC, and includes nucleic acid sequence SEQ ID NO:43, which encodes a χ subunit protein including SEQ ID NO:45. Another preferred Y. pestis gene is holD, and includes nucleic acid sequence SEQ ID NO:48, which encodes a ψ subunit protein including SEQ ID NO:50. Another preferred Y. pestis gene is ssb, and includes nucleic acid sequence SEQ ID NO:53, which encodes a ψ subunit protein including SEQ ID NO:55. Another preferred Y. pestis gene is dnaG, and includes nucleic acid sequence SEQ ID NO:58, which encodes a dnaG subunit protein including SEQ ID NO:60.

It should be noted that since nucleic acid sequencing technology is not entirely error-free, sequences presented herein, at best, represent an apparent nucleic acid sequence of the nucleic acid molecules encoding a P. aeruginosa or Y. pestis DNA polymerase holoenzyme subunit protein of the present invention. A nucleic acid molecule of the present invention can include an isolated natural gene or a homolog thereof, the latter of which is described in more detail below. A nucleic acid molecule of the present invention can include one or more regulatory regions, full-length or partial coding regions, or combinations thereof. The minimal size of a nucleic acid molecule of the present invention is the minimal size that can form a stable hybrid with one of the aforementioned genes under stringent hybridization conditions.

In one embodiment, hybridization conditions will permit isolation of nucleic acid molecules having at least about 70% nucleic acid sequence identity with the nucleic acid molecule being used to probe. In preferred embodiments, hybridization conditions will permit isolation of nucleic acid molecules having at least about 80% nucleic acid sequence identity with the nucleic acid molecule being used to probe. In more preferred embodiments, hybridization conditions will permit isolation of nucleic acid molecules having at least about 90% nucleic acid sequence identity with the nucleic acid molecule being used to probe. In more preferred embodiments, hybridization conditions will permit isolation of nucleic acid molecules having at least about 95% nucleic acid sequence identity with the nucleic acid molecule being used to probe.

In accordance with the present invention, an isolated nucleic acid molecule is a nucleic acid molecule that is identified and separated from at least one contaminant nucleic acid with which it is ordinarily associated in its natural source. Isolated nucleic acid molecules are thus present in a form or setting that is different from that in which it is found in nature. In contrast, non-isolated nucleic acids as nucleic acids such as DNA and RNA found in the state they exist in nature. The isolated nucleic acid moleucle may be present in single-stranded or double-stranded form. When an isolated nucleic acid moleucule is to be utilized to express a protein, the nucleic acid moleucule will contain at a minimum the sense or coding strand (i. e., the oligonucleotide or polynucleotide may single-stranded), but may contain both the sense and anti-sense strands (i. e., the oligonucleotide or polynucleotide may be double-stranded). “Isolated” does not reflect the extent to which the nucleic acid molecule has been purified. An isolated nucleic acid molecule can include DNA, RNA, or derivatives of either DNA or RNA.

An isolated P. aeruginosa or Y. pestis DNA polymerase III holoenzyme subunit nucleic acid molecule of the present invention can be obtained from its natural source either as an entire (i.e., complete) gene or a portion thereof capable of forming a stable hybrid with that gene. An isolated P. aeruginosa or Y. pestis DNA polymerase III holoenzyme subunit nucleic acid molecule can also be produced using recombinant DNA technology (e.g., polymerase chain reaction (PCR) amplification, cloning) or chemical synthesis. Isolated P. aeruginosa or Y. pestis DNA polymerase III holoenzyme subunit nucleic acid molecules include natural nucleic acid molecules and homologs thereof, including, but not limited to, natural allelic variants and modified nucleic acid molecules in which nucleotides have been inserted, deleted, substituted, and/or inverted in such a manner that such modifications do not substantially interfere with the nucleic acid molecule's ability to encode a protein of the present invention or to form stable hybrids under stringent conditions with natural gene isolates.

A P. aeruginosa or Y. pestis DNA polymerase III holoenzyme subunit nucleic acid molecule homolog can be produced using a number of methods known to those skilled in the art (see, for example, Sambrook et al., ibid.). For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologs can be selected from a mixture of modified nucleic acids by screening for the function of the protein encoded by the nucleic acid (e.g., ability to elicit an immune response against at least one epitope of a P. aeruginosa or Y. pestis DNA polymerase III holoenzyme subunit protein, ability to bind to immune serum) and/or by hybridization with a P. aeruginosa or Y. pestis DNA polymerase III holoenzyme subunit gene.

The present invention also provides methods for detection of nucleic acid molecules encoding at least a portion of DNA polymerase III holoenzyme, or DNA polymerase III holoenzyme subunit in a biological sample comprising the steps of: a) hybridizing at least a portion of a nucleic acid molecule of the present invention to nucleic acid material of a biological sample, thereby forming a hybridization complex, and b) detecting the hybridization complex, wherein the presence of the complex correlates with the presence of a polynucleotide encoding at least a portion of DNA polymerase III holoenzyme or DNA polymerase III holoenzyme subunit in the biological sample. In preferred embodiments, hybridization conditions will permit isolation of nucleic acid molecules having at least about 80% nucleic acid sequence identity with the nucleic acid molecule being used to probe. In more preferred embodiments, hybridization conditions will permit isolation of nucleic acid molecules having at least about 90% nucleic acid sequence identity with the nucleic acid molecule being used to probe. In more preferred embodiments, hybridization conditions will permit isolation of nucleic acid molecules having at least about 95% nucleic acid sequence identity with the nucleic acid molecule being used to probe. In alternative preferred embodiment of the methods, the nucleic acid material of the biological sample is amplified by the polymerase chain reaction.

Recombinant Molecules, Recombinant Cells, and Uses of Recombinant Molecules and Cells

The present invention also includes a recombinant vector, which includes at least one P. aeruginosa or Y. pestis DNA polymerase III nucleic acid molecule of the present invention, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid. Recombinant vectors can be used in the cloning, sequencing, and/or otherwise manipulating of P. aeruginosa or Y. pestis DNA polymerase III nucleic acid molecules of the present invention. One type of recombinant vector, referred to herein as a recombinant molecule and described in more detail below, can be used in the expression of nucleic acid molecules of the present invention. Preferred recombinant vectors are capable of replicating in the transformed cell.

Isolated P. aeruginosa or Y. pestis DNA polymerase III proteins of the present invention can be produced in a variety of ways, including production and recovery of natural proteins, production and recovery of recombinant proteins, and chemical synthesis of the proteins. In one embodiment, an isolated protein of the present invention is produced by culturing a cell capable of expressing the protein under conditions effective to produce the protein, and recovering the protein. A preferred cell to culture is a recombinant cell that is capable of expressing the protein, the recombinant cell being produced by transforming a host cell with one or more nucleic acid molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained. Suitable and preferred nucleic acid molecules with which to transform a cell are as disclosed herein for suitable and preferred P. aeruginosa or Y. pestis DNA polymerase III nucleic acid molecules per se. Particularly preferred nucleic acid molecules to include in recombinant cells of the present invention include P. aeruginosa and Y. pestis dnaN, dnaE, dnaX, dnaQ, holA, holB, holC, holD, holE, ssb, and dnaG.

Suitable host cells to transform include any cell that can be transformed with a nucleic acid molecule of the present invention. Host cells can be either untransformed cells or cells that are already transformed with at least one nucleic acid molecule. Host cells of the present invention either can be endogenously (i.e., naturally) capable of producing P. aeruginosa or Y. pestis DNA polymerase III proteins of the present invention or can be capable of producing such proteins after being transformed with at least one nucleic acid molecule of the present invention. Host cells of the present invention can be any cell capable of producing at least one protein of the present invention, and include bacterial, fungal (including yeast), insect, other animal and plant cells. Preferred host cells include bacterial, mycobacterial, yeast, insect and mammalian cells. More preferred host cells include Escherichia. Particularly preferred host cells are Escherichia coli, including DH5α, MGC1030 and AP1.L1 strains. Alternative preferred host cells are P. aeruginosa or Y. pestis, including attenuated strains with reduced pathogenicity.

A recombinant cell is preferably produced by transforming a host cell with one or more recombinant molecules, each comprising one or more nucleic acid molecules of the present invention operatively linked to an expression vector containing one or more transcription control sequences. The phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell and of effecting expression of a specified nucleic acid molecule. The term “vehicle” is sometimes used interchangeably with “vector.” Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, parasite, insect, other animal, and plant cells. Preferred expression vectors of the present invention can direct gene expression in bacterial, yeast, insect and mammalian cells and more preferably in the cell types heretofore disclosed.

Recombinant molecules of the present invention may also (a) contain secretory signals (i.e., signal segment nucleic acid sequences) to enable an expressed P. aeruginosa or Y. pestis protein of the present invention to be secreted from the cell that produces the protein and/or (b) contain fusion sequences which lead to the expression of nucleic acid molecules of the present invention as fusion proteins. Eukaryotic recombinant molecules may include intervening and/or untranslated sequences surrounding and/or within the nucleic acid sequences of nucleic acid molecules of the present invention.

Suitable signal segments include natural signal segments or any heterologous signal segment capable of directing the secretion of a protein of the present invention. Preferred signal segments include, but are not limited to, tissue plasminogen activator (t-PA), interferon, interleukin, growth hormone, histocompatibility and viral envelope glycoprotein signal segments.

Nucleic acid molecules of the present invention can be operatively linked to expression vectors containing regulatory sequences such as transcription control sequences, translation control sequences, origins of replication, and other regulatory sequences that are compatible with the recombinant cell and that control the expression of nucleic acid molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, insect and mammalian cells, such as, but not limited to, pA1, tac, lac, trp, trc, oxy-pro, omp/lpp, rrnB, bacteriophage lambda (λ), bacteriophage T7, T7lac, bacteriophage T3, bacteriophage SP6, bacteriophage SP01, metallothionein, α-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells. Additional suitable transcription control sequences include tissue-specific promoters and enhancers as well as lymphokine-inducible promoters (e.g., promoters inducible by interferons or interleukins). Transcription control sequences of the present invention can also include naturally occurring transcription control sequences naturally associated with P. aeruginosa or Y. pestis.

A recombinant molecule of the present invention is a molecule that can include at least one of any nucleic acid molecule heretofore described operatively linked to at least one of any transcription control sequence capable of effectively regulating expression of the nucleic acid molecule(s) in the cell to be transformed. A recombinant cell of the present invention includes any cell transformed with at least one of any nucleic acid molecule of the present invention. Recombinant cells of the present invention can also be co-transformed with one or more recombinant molecules including P. aeruginosa or Y. pestis DNA Polymerase III nucleic acid molecules encoding one or more proteins of the present invention, examples of which are disclosed herein. Particularly preferred recombinant molecules include pA1-YP-dnaE5′, pA1-YP-dnaE, pA1-YP-dnaQ, pA1-YP-holE, pA1-YP-QE, pA1-YP-core, pA1-YP-dnaN, pA1-YP-dnaX, pA1-YP-holA, pA1-YP-holB, pA1-YP-holC, pA1-YP-holD, pA1-YP-holCD, pA1-YP-holBA, pA1-YP-holBAX, pA1-YP-CLcomplex, pA1-YP-ssb, pA1-YP-dnaG, pA1-PA-dnaE#15′, pA1-PA-dnaE#1, pA1-PA-dnaE#2-5′, pA1-PA-dnaE#2, pA1-PA-dnaQ, pA1-PA-core#1, pA1-PA-core#2, pA1-PA-dnaX, pA1-PA-holA, pA1-PA-holB, pA1-NB-PAholC, pUCP19-NB-PAholC, pA1-CB-PAholC, pUCP19-CB-PAholC, pA1-PA-holBA, pA1-PA-BAX, pA1-PA-dnaG, and pA1-PA-ssb. Details regarding the production of such recombinant molecules are disclosed herein.

A recombinant cell of the present invention includes any cell transformed with at least one of any nucleic acid molecule of the present invention. Suitable and preferred nucleic acid molecules as well as suitable and preferred recombinant molecules with which to transfer cells are disclosed herein. Preferred recombinant cells include Escherichia coli, including DH5α, MGC1030 and AP1.L1 strains, P. aeruginosa strains, and Y. pestis strains which have been transformed with the preferred recombinant molecules listed above. Particularly preferred recombinant cells include transformed cells described in the Examples section below. Details regarding the production of these recombinant cells are disclosed herein.

Recombinant cells of the present invention can also be co-transformed with one or more recombinant molecules including PsuDNA Polymerase III nucleic acid molecules encoding one or more proteins of the present invention.

It may be appreciated by one skilled in the art that use of recombinant DNA technologies can improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules of the present invention to correspond to the codon usage of the host cell, deletion of sequences that destabilize transcripts, and use of control signals that temporally separate recombinant cell growth from recombinant enzyme production during fermentation. The activity of an expressed recombinant protein of the present invention may be improved by fragmenting, modifying, or derivatizing nucleic acid molecules encoding such a protein.

Recombinant cells of the present invention can be used to produce one or more proteins of the present invention by culturing such cells under conditions effective to produce such a protein, and recovering the protein. Effective conditions to produce a protein include, but are not limited to, appropriate media, bioreactor, temperature, pH and oxygen conditions that permit protein production. An appropriate, or effective, medium refers to any medium in which a cell of the present invention, when cultured, is capable of producing a P. aeruginosa or Y. pestis DNA Polymerase III protein of the present invention. Such a medium is typically an aqueous medium comprising assimilable carbon, nitrogen and phosphate sources, as well as appropriate salts, minerals, metals and other nutrients, such as vitamins. The medium may comprise complex nutrients or may be a defined minimal medium. Cells of the present invention can be cultured in conventional fermentation bioreactors, which include, but are not limited to, batch, fed-batch, cell recycle, and continuous fermentors. Culturing can also be conducted in shake flasks, test tubes, microtiter dishes, and petri plates. Culturing is carried out at a temperature, pH and oxygen content appropriate for the recombinant cell. Such culturing conditions are well within the expertise of one of ordinary skill in the art.

Depending on the vector and host system used for production, resultant proteins of the present invention may either remain within the recombinant cell; be secreted into the fermentation medium; be secreted into a space between two cellular membranes, such as the periplasmic space in E. coli; or be retained on the outer surface of a cell or viral membrane.

The phrase “recovering the protein” refers simply to collecting the whole fermentation medium containing the protein and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. Proteins of the present invention are preferably retrieved in “substantially pure” form. As used herein, “substantially pure” refers to a purity that allows for the effective use of the protein as a therapeutic composition or diagnostic.

Antibodies

The present invention also includes isolated antibodies capable of selectively binding to a P. aeruginosa or Y. pestis DNA Polymerase III holoenzyme subunit protein of the present invention or to a mimetope thereof. Such antibodies are also referred to herein as anti-P. aeruginosa or anti-Y. pestis DNA Polymerase III holoenzyme subunit antibodies. Particularly preferred antibodies of this embodiment include anti-DnaE antibodies, anti-DnaN antibodies, anti-DnaX antibodies, anti-HolA and anti-HolB antibodies.

Isolated antibodies are antibodies that have been removed from their natural milieu. The term “isolated” does not refer to the state of purity of such antibodies. As such, isolated antibodies can include anti-sera containing such antibodies, or antibodies that have been purified to varying degrees.

As used herein, the term “selectively binds to” refers to the ability of antibodies of the present invention to preferentially bind to specified proteins and mimetopes thereof of the present invention. Binding can be measured using a variety of methods known to those skilled in the art including immunoblot assays, immunoprecipitation assays, radioimmunoassays, enzyme immunoassays (e.g., ELISA), immunofluorescent antibody assays and immunoelectron microscopy; see, for example, Sambrook et al., ibid.

Antibodies of the present invention can be either polyclonal or monoclonal antibodies. Antibodies of the present invention include functional equivalents such as antibody fragments and genetically-engineered antibodies, including single chain antibodies, that are capable of selectively binding to at least one of the epitopes of the protein or mimetope used to obtain the antibodies. Antibodies of the present invention also include chimeric antibodies that can bind to more than one epitope. Preferred antibodies are raised in response to proteins, or mimetopes thereof, that are encoded, at least in part, by a nucleic acid molecule of the present invention. Methods to generate and detect antibodies are known in the art. See, e.g., Harlow and Lane, Antibodies: A Laboratory Manual, Cold Sprng Harbor Laboratory Press, Cold Spring Harbor, N.Y., incorporated by reference herein in its entirety.

A preferred method to produce antibodies of the present invention includes (a) administering to an animal an effective amount of a protein or mimetope thereof of the present invention to produce the antibodies and (b) recovering the antibodies. In another method, antibodies of the present invention are produced recombinantly using techniques as heretofore disclosed to produce P. aeruginosa or Y. pestis DNA Polymerase III holoenzyme subunit proteins of the present invention. Antibodies raised against defined proteins or mimetopes can be advantageous because such antibodies are not substantially contaminated with antibodies against other substances that might otherwise cause interference in a diagnostic assay or side effects if used in a therapeutic composition.

Antibodies of the present invention have a variety of potential uses that are within the scope of the present invention. For example, such antibodies can be used (a) as therapeutic compounds to passively immunize an animal in order to protect the animal from bacteria susceptible to treatment by such antibodies, preferably P. aeruginosa or Y. pestis, (b) as reagents in assays to detect infection by such bacteria and/or (c) as tools to screen expression libraries and/or to recover desired proteins of the present invention from a mixture of proteins and other contaminants. Furthermore, antibodies of the present invention can be used to target cytotoxic agents to bacteria of the present invention in order to directly kill such bacteria. Targeting can be accomplished by conjugating (i.e., stably joining) such antibodies to the cytotoxic agents using techniques known to those skilled in the art. Suitable cytotoxic agents are known to those skilled in the art. Suitable cytotoxic agents include, but are not limited to, all classes of antibacterial drugs.

Detection of Amino Acid Molecules and Nucleic Acid Molecules of the Present Invention

The present invention also provides methods for detecting DNA polymerase III comprising: providing in any order, a sample suspected of containing DNA polymerase III, and an antibody capable of specifically binding to at least a portion of the DNA polymerase III; mixing the sample and the antibody under conditions wherein the antibody can bind to the DNA polymerase III; and detecting the binding. In alternative preferred embodiments, the organism is P. aeruginosa or Y. pestis. Methods for detecting proteins with antibodies are well known to those skilled in the art, see, for example Harlow and Lane, ibid., and include immunoblot assays, immunoprecipitation assays, enzyme immunoassays (e.g., ELISA), radioimmunoassays, immunofluorescent antibody assays and immunoelectron microscopy.

The present invention also provides methods for detection of nucleic acid molecules encoding at least a portion of DNA polymerase III holoenzyme, or DNA polymerase III holoenzyme subunit in a biological sample comprising the steps of: a) hybridizing at least a portion of a nucleic acid molecule of the present invention to nucleic acid material of a biological sample, thereby forming a hybridization complex, and b) detecting the hybridization complex, wherein the presence of the complex correlates with the presence of a polynucleotide encoding at least a portion of DNA polymerase III holoenzyme or DNA polymerase III holoenzyme subunit in the biological sample. In alternative preferred embodiment of the methods, the nucleic acid material of the biological sample is amplified by the polymerase chain reaction.

The present invention also provides methods for detecting DNA polymerase III holoenzyme or holoenzyme subunit expression, including expression of abnormal or mutated DNA polymerase III holoenzyme or holoenzyme subunint proteins or gene sequences comprising the steps of a) providing a test sample suspected of containing DNA polymerase III holoenzyme or DNA polymerase III holoenzyme subunit protein, as appropriate; and b) comparing the test DNA polymerase III holoenzyme or holoenzyme subunit, in the sample with the quantitated DNA polymerase III holoenzyme or holoenzyme subunit in the control to determine the relative concentration of the test DNA polymerase III holoenzyme or holoenzyme subunit in the sample. In addition, the methods may be conducted using any suitable means to determine the relative concentration of DNA polymerase holoenzyme or holoenzyme subunit in the test and control samples. Examples of such methods may be found in the Examples section.

Another embodiment of the present invention is a method for detecting functional activity of P. aeruginosa or Y. pestis DNA polymerase III protein subunits. A preferred method is the detection of activity comprising a) providing a test sample suspected of containing DNA polymerase III holoenzyme subunit protein; and b) comparing the activity of the test holoenzyme subunit in the sample with a quantitated DNA polymerase III holoenzyme subunit in a control to determine the relative activity of the test DNA polymerase III holoenzyme subunit in the sample. In one embodiment the activity is polymerase gap-filling activity for the detection of DNA polymerase III type DnaE subunit. In another embodiment, the activity is the stimulation of the processitivity of the DNA polymerase for detection of the DnaN subunit. In another embodiment the activity is the DNA-dependent ATPase activity of the DnaX subunit. In another embodiment, the activity is rapid and processive DNA synthesis on RNA- or DNA-primed long single-stranded DNA template in the presence of DnaE, DnaN, DnaX, HolA and HolB.

Modulation of the Function of Replicases

The minimal assembly of the essential subunits of a bacterial replicase should permit rapid and processive synthesis of long stretches of DNA. In all bacterial replicases studied so far, the minimal functional holoenzyme consists of three components: 1) the polymerase core, 2) the β processivity factor-loading (or clamp-loading) complex and 3) the sliding clamp processivity factor, β. In E. coli, the minimal holoenzyme consists of the a subunit, DnaX-δδ clamp loading complex and the β subunit. The same components are minimally required for functional DNA Pol III holoenzyme from Gram-positive S. pyogenes and T. thermophilus. Given the generality of this requirement among distantly related organisms, it is expected that α, β, DnaX, δ, and δ′ will be sufficient for the assembly of a replicase for most organisms, including Y. pestis and P. aeruginosa. As used herein, DnaX complex is the term used to describe an assembly of DnaX, delta and delta′.

The present invention includes the use of modulators or suspected modulators of bacterial DNA replication that modulate the replication activity of a DNA polymerase III replicase to reduce bacterial infection of animals, plants, humans and the surrounding environment. As used herein, modulators of bacterial DNA replication are compounds that interact with the replicase thereby altering the ability of replicase to replicate DNA. A preferred modulator inhibits replication.

Target indications may include arresting cell growth or causing cell death resulting in recovery from the bacterial infection in animal studies. A wide variety of assays for activity and binding agents are provided, including DNA synthesis, ATPase, clamp loading onto DNA, protein-protein binding assays, immunoassays, cell based assays, etc. The replication protein compositions, used to identify pharmacological agents, are in isolated, partially pure or pure form and are typically, but not necessarily, recombinantly produced. The replication protein may be part of a fusion product with another peptide or polypeptide (e. g., a polypeptide that is capable of providing or enhancing protein-protein binding, stability under assay conditions (e. g., a tag for detection or anchoring), etc.). The assay mixtures comprise a natural intracellular replication protein-binding target such as DNA, another protein, NTP, or dNTP. For binding assays, while native binding targets may be used, it is frequently preferred to use portions (e.g., peptides, nucleic acid fragments) thereof so long as the portion provides binding affinity and avidity to the subject replication protein conveniently measurable in the assay. The assay mixture also comprises a candidate pharmacological agent. Generally, a plurality of assay mixtures are run in parallel with different agent concentrations to obtain a differential response to the various concentrations. Typically, one of these concentrations serves as a negative control (i.e., at zero concentration or below the limits of assay detection). Additional controls are often present such as a positive control, a dose response curve, use of known inhibitors, use of control heterologous proteins, etc. Candidate agents encompass numerous chemical classes, though typically they are organic compounds; preferably they are small organic compounds and are obtained from a wide variety of sources, including libraries of synthetic or natural compounds. A variety of other reagents may also be included in the mixture. These include reagents like salts, buffers, neutral proteins (e.g., albumin, detergents, etc.) which may be used to facilitate optimal binding and/or reduce nonspecific or background intersactions, etc. Also, reagents that otherwise improve the efficiency of the assay (e.g., protease inhibitors, nuclease inhibitors, antimicrobial agents, etc.) may be used.

The invention provides replication protein specific assays and the binding agents including natural intracellular binding targets such as other replication proteins, etc., and methods of identifying and making such agents, and their use in a a variety of diagnostic and therapeutic applications, especially where disease is associated with excessive cell growth. Novel replication protein-specific antibodies and other natural intracellular binding agents identified with assays such as one- and two-hybrid screens, non-natural intracellular binding agents identified in screens of chemical libraries, etc.

Generally, replication protein-specificity of the binding agent is shown by equilibrium constants or values that approximate equilibrium constants such as the concentration that inhibits a reaction 50% (IC₅₀). Such agents are capable of selectively binding a replication protein (i.e., with an equilibrium association constant at least about 10⁷ M⁻¹, preferably, at least about 10⁸ M⁻¹, more preferably, at least about 10⁹ M⁻¹). A wide variety of cell-based and cell-free assays may be used to demonstrate replication protein-specific activity, binding, gel shift assays, immunoassays, etc.

The resultant mixture is incubated under conditions whereby, but for the presence of the candidate pharmacological agent, the replication protein specifically binds the cellular binding target, portion, or analog. The mixture of components can be added in any order that provides for the requisite bindings.

Incubations may be performed at any temperature which facilitates optimal binding, typically between 4° C. and 40° C., more commonly between 15° C. and 37° C. Incubation periods are likewise selected for optimal binding but also minimized to facilitate rapid, high-throughput screening, and are typically between 0.01 and 10 hours, preferably less than 5 hours, more preferably less than 1 hour.

After incubation, the presence or absence of activity or specific binding between the replication protein and one or more binding targets is detected by any convenient way. For cell-free activity and binding type assays, a separation step may be used to separate the activity product or the bound from unbound components.

Separation may be effected by precipitation (e. g., immunoprecipitation or acid precipitation of a nucleic acid product), immobilization (e. g., on a solid substrate such as a microtiter plate), etc., followed by washing. Many assays that do not require separation are also possible such as use of europium conjugation in proximity assays, fluorescence increases upon double stranded DNA synthesis in the presence of dyes that increase fluorescence upon intercalating in double-stranded DNA, scintillation proximity assays or a detection system that is dependent on a reaction product or loss of substrate.

Detection may be effected in any convenient way. For cell-free activity and binding assays, one of the components usually comprises or is coupled to a label.

A wide variety of labels may be employed-essentially any label that provides for detection of DNA product, loss of DNA substrate, conversion of a nucleotide substrate, or bound protein is useful. The label may provide for direct detection such as radioactivity, fluorescence, luminescence, optical, or electron density, etc. or indirect detection such as an epitope tag, an enzyme, etc. The label may be appended to the protein (e.g., a phosphate group comprising a radioactive isotope of phosphorous), or incorporated into the DNA substrate or the protein structure (e.g., a methionine residue comprising a radioactive isotope of sulfur). A variety of methods may be used to detect the label depending on the nature of the label and other assay components. For example, the label may be detected bound to the solid substrate, or a portion of the bound complex containing the label may be separated from the solid substrate, and thereafter the label detected. Labels may be directly detected through optical or electron density, radioactive emissions, nonradiative energy transfer, fluorescence emission, etc. or indirectly detected with antibody conjugates, etc. For example, in the case of radioactive labels, emissions may be detected directly (e.g., with particle counters) or indirectly (e.g., with scintillation cocktails and counters).

The present invention provides methods by which replication proteins from bacteria, preferably Y. pestis and P. aeruginosa, are used to discover new pharmaceutical agents. The function of replication proteins is quantified in the presence of different chemical test compounds. A chemical compound that inhibits the function is a candidate antibiotic.

In some embodiments it is prefereable to use replication protein subunits derived from a single organism. In other embodiments, the various subunits may be derived from one or more organisms. Replication proteins from Gram positive bacteria and from Gram negative bacteria can be interchanged for one another in certain embodiments. Hence, they can function as mixtures. Suitable E. coli replication proteins are the subunits of its Pol III holoenzyme which are described in U. S. Pat. Nos. 5,583,026 and 5,668,004 to O'Donnell, which are hereby incorporated by reference.

Some other replication subunit proteins useful in the present invention are detailed in copending U.S. patent application Ser. No. 09/906,179, especially Tables 3-18 and 21, filed Jul. 16, 2001, entitled “Novel DNA Polymerase III Holoenzyme Delta Subunit Nucleic Acid Molecules And Proteins,” incorporated by reference herein in its entirety.

The present invention, in one embodiment provides a method of screening for a compound or chemical that modulates the activity of a DNA polymerase III replicase comprising, a) contacting an isolated replicase with at least one test compound under conditions permissive for replicase activity, b) assessing the activity of the replicase in the presence of the test compound, and c) comparing the activity of the replicase in the presence of the test compound with the activity of the replicase in the absence of the test compound, wherein a change in the activity of the replicase in the presence of the test compound is indicative of a compound that modulates the activity of the replicase. In preferred embodiments, the replicase comprises Y. pestis or P. aeruginosa replicase subunit proteins.

The present invention describes a method of identifying compounds which modulate the activity of a DNA polymerase III replicase. This method is carried out by forming a reaction mixture that includes a template DNA molecule, a DnaG primase, SSB, a DNA polymerase III replicase, a candidate compound, a mixture of four NTPs and dNTPs, and optionally either a β subunit, a DnaX complex, or both the β subunit and the DnaX complex; subjecting the reaction mixture to conditions effective to achieve nucleic acid polymerization in the absence of the candidate compound; analyzing the reaction mixture for the presence or absence of nucleic acid polymerization extension products and comparing the activity of the replicase in the presence of the test compound with the activity of the replicase in the absence of the test compound. In alternative embodiments, DnaG and SSB can be omitted, and the the template DNA molecule can be primed with an oligonucleotide primer. A change in the activity of the replicase in the presence of the test compound is indicative of a compound that modulates the activity of the replicase. In preferred embodiments, the DnaX complex comprises Y. pestis or P. aeruginosa DnaX complex subunit proteins.

The present invention describes a method to identify candidate pharmaceuticals that modulate the activity of a DnaX complex and a β subunit in stimulating the DNA polymerase. The method includes contacting a primed DNA (which may be coated with SSB) with a DNA polymerase, a β subunit, and a DnaX complex (or subunit or subassembly of the DnaX complex) in the presence of the candidate pharmaceutical, and dNTPs (or modified dNTPs) to form a reaction mixture. The reaction mixture is subjected to conditions which, in the absence of the candidate pharmaceutical, would effect nucleic acid polymerization and the presence or absence of the extension product in the reaction mixture is analyzed. The nucleic acid polymerization in the presence of the test compound is compared with the nucleic acid polymerization in the absence of the test compound. A change in the nucleic acid polymerization in the presence of the test compound is indicative of a compound that modulates the activity of a DnaX complex and a β subunit. In preferred embodiments, the DnaX complex comprises Y. pestis or P. aeruginosa DnaX complex subunit proteins, and/or the β subunit is a Y. pestis or P. aeruginosa β subunit.

The present invention describes a method to identify chemicals that modulate the ability of a β subunit and a DnaX complex (or a subunit or subassembly of the DnaX complex) to interact. This method includes contacting the β subunit with the DnaX complex (or subunit or subassembly of the DnaX complex) in the presence of ATP (or a suitable ATP analog, such as ATPγS, a non-hydrolyzable analog of ATP), that enables interaction of β with the DnaX complex (or subunit or subassembly of the DnaX complex) the candidate pharmaceutical to form a reaction mixture. The reaction mixture is subjected to conditions under which the DnaX complex (or the subunit or subassembly of the DnaX complex) and the β subunit would interact in the absence of the candidate pharmaceutical. The reaction mixture is then analyzed for interaction between the β subunit and the DnaX complex (or the subunit or subassembly of the DnaX complex). The extent of interaction in the presence of the test compound is compared with the extent of interaction in the absence of the test compound. A change in the interaction between the β subunit and the DnaX complex (or the subunit or subassembly of the DnaX complex) is indicitive of a compound that modulates the interaction. In preferred embodiments, the DnaX complex comprises Y. pestis or P. aeruginosa DnaX complex subunit proteins, and/or the β subunit is a Y. pestis or P. aeruginosa β subunit.

The present invention describes a method to identify chemicals that modulate the ability of a δ subunit and the δ′ and/or DnaX subunit to interact. This method includes contacting the δ subunit with the δ′ and/or δ′ plus DnaX subunit in the presence of the candidate pharmaceutical to form a reaction mixture. The reaction mixture is subjected to conditions under which the δ subunit and the δ′ and/or δ′ plus DnaX subunit would interact in the absence of the candidate pharmaceutical. The reaction mixture is then analyzed for interaction between the δ subunit and the δ and/or DnaX subunit. The extent of interaction in the presence of the test compound is compared with the extent of interaction in the absence of the test compound. A change in the interaction between the δ subunit and the δ′ and/or DnaX subunit is indicitive of a compound that modulates the interaction. In preferred embodiments, the δ,δ′, and DnaX subunits are Y. pestis and/or P. aeruginosa subunits.

The present invention describes a method to identify chemicals that modulate the ability of a DnaX complex (or a subassembly of the DnaX complex) to assemble a β subunit onto a DNA molecule. This method involves contacting a circular primed DNA molecule (which may be coated with SSB) with the DnaX complex (or the subassembly thereof) and the β subunit in the presence of the candidate pharmaceutical, and ATP or dATP to form a reaction mixture. The reaction mixture is subjected to conditions under which the DnaX complex (or subassembly) assembles the β subunit on the DNA molecule absent the candidate pharmaceutical. The presence or absence of the β subunit on the DNA molecule in the reaction mixture is analyzed. The extent of assembly in the presence of the test compound is compared with the extent of assmbly in the absence of the test compound. A change in the amount of β subunit on the DNA molecule is indicative of a compound that modulates the ability of a DnaX complex (or a subassembly of the DnaX complex) to assemble a β subunit onto a DNA molecule. In preferred embodiments, the DnaX complex or subassembly thereof comprises Y. pestis or P. aeruginosa DnaX complex subunit proteins, and/or the β subunit is a Y. pestis or P. aeruginosa β subunit.

The present invention describes a method to identify chemicals that modulate the ability of a DnaX complex (or subunit(s) of the DnaX complex) to disassemble a β subunit from a DNA molecule. This method comprises contacting a DNA molecule onto which the β subunit has been assembled in the presence of the candidate pharmaceutical, to form a reaction mixture. The reaction mixture is subjected to conditions under which the DnaX complex (or subunit(s) or subassembly of the DnaX complex) disassembles the β subunit from the DNA molecule absent the candidate pharmaceutical. The presence or absence of the β subunit on the DNA molecule in the reaction mixture is analyzed. The extent of assembly in the presence of the test compound is compared with the extent of assembly in the absence of the test compound. A change in the amount of β subunit on the DNA molecule is indicative of a compound that modulates the ability of a DnaX complex (or a subassembly of the DnaX complex) to disassemble a β subunit onto a DNA molecule. In preferred embodiments, the DnaX complex or subassembly comprises Y. pestis or P. aeruginosa DnaX complex subunit proteins, and/or the β subunit is a Y. pestis or P. aeruginosa β subunit.

The present invention describes a method to identify chemicals that modulate the dATP/ATP binding activity of a DnaX complex or a DnaX complex subunit (e.g. DnaX subunit). This method includes contacting the DnaX complex (or the DnaX complex subunit) with dATP/ATP either in the presence or absence of a DNA molecule and/or the β subunit in the presence of the candidate pharmaceutical to form a reaction. The reaction mixture is subjected to conditions in which the DnaX complex (or the subunit of DnaX complex) interacts with dATP/ATP in the absence of the candidate pharmaceutical. The reaction is analyzed to determine if dATP/ATP is bound to the DnaX complex (or the subunit of DnaX complex) in the presence of the candidate pharmaceutical. The extent of binding in the presence of the test compound is compared with the extent of binding in the absence of the test compound. A change in the dATP/ATP binding is indicative of a compound that modulates the dATP/ATP binding activity of a DnaX complex or a DnaX complex subunit (e.g. DnaX subunit). In preferred embodiments, the DnaX complex or subassembly comprises Y. pestis or P. aeruginosa DnaX complex subunit proteins.

The present invention describes a method to identify chemicals that modulate the dATP/ATPase activity of a DnaX complex or a DnaX complex subunit (e.g., the DnaX subunit). This method involves contacting the DnaX complex (or the DnaX complex subunit) with dATP/ATP either in the presence or absence of a DNA molecule and/or a β subunit in the presence of the candidate pharmaceutical to form a reaction mixture. The reaction mixture is subjected to conditions in which the DnaX subunit (or complex) hydrolyzes dATP/ATP in the absence of the candidate pharmaceutical. The reaction is analyzed to determine if dATP/ATP was hydrolyzed. The extent of hyrdolysis in the presence of the test compound is compared with the extent of hydrolysis in the absence of the test compound. A change in the amount of dATP/ATP hydrolyzed is indicative of a compound that modulates the dATP/ATPase activity of a DnaX complex or a DnaX complex subunit (e.g., the DnaX subunit). The DnaX complex comprises a δ protein of the present invention. In preferred embodiments, the DnaX complex or subassembly comprises Y. pestis or P. aeruginosa DnaX complex subunit proteins.

The invention provides a further method for identifying chemicals that modulate the activity of a DNA polymerase comprising contacting a circular primed DNA molecule (may be coated with SSB) with a DnaX complex, a β subunit and an a subunit in the presence of the candidate pharmaceutical, and dNTPs (or modified dNTPs) to form a reaction mixture. The reaction mixture is subjected to conditions, which in the absence of the candidate pharmaceutical, affect nucleic acid polymerization, and the presence or absence of the extension product in the reaction mixture is analyzed. The activity of the replicase in the presence of the test compound is compared with the activity of the replicase in the absence of the test compound. A change in the activity of the replicase in the presence of the test compound is indicative of a compound that modulates the activity of the replicase. In preferred embodiments, the replicase comprises Y. pestis or P. aeruginosa replicase subunit proteins.

The present invention also includes a test kit to identify a compound capable of modulating DNA polymerase III replicase activity. Such a test kit includes an isolated replicase subunit protein from Y. pestis or P. aeruginosa and a means for determining the extent of modulation of DNA replication activity in the presence of (i.e., effected by) a putative inhibitory compound.

The present invention also includes modulators and inhibitors isolated by such a method, and/or test kit, and their use to inhibit any replicase of the present invention that is susceptible to such an inhibitor. Modulators of bacterial DNA replication can also be used directly as to treat animals, plants and humans suspected of having a bacterial infection as long as such compounds are not harmful to the species being treated. Similarly, molecules that bind to any replicase of the present invention, incuding but not limited to antibodies and small molecules, can also be used to target cytotoxic, therapeutic or imaging entities to a site of infection.

With the availability of the essential DNA polymerase III holoenzyme subunits purified by a reconstitution approach, replication methods of the invention are amenable to a high-throughput screen against the new target provided. This can be done by titrating each component until it is barely limiting so that modulation will be observed upon interaction with active compounds within small molecule libraries. Buffer conditions are optimized to ensure stability of the enzyme mix and stored in a separate chilled container during the multi-plate screening process. The time and temperature of the assay are also optimized to ensure a stable linear response. Similarly, concentrations of all the relevant components of the assay are optimized to ensure high sensitivity of the assay to perturbation by test compounds.

The present invention also provides methods for screening antibacterial drug candidates that modulate replicase activity of P. aeruginosa or Y. pestis DNA polymerase holoenzyme. This method comprises the steps of a) providing an test inhibitor suspected of modulating DNA polymerase III holoenzyme replication, b) detecting the DNA polymerase III replication reaction in test and control reaction, and c) comparing the test to the control, wherein the amount of replication correlates with the modulatory effect of the test inhibitor. The present invention also provides a corresponding method for screening antibacterial drug candidates that modulate or inhibit the activity of P. aeruginosa or Y. pestis primosome. Examples of such methods may be found in the Examples section.

This invention provides methods for the assembly of a complete P. aeruginosa and Y. pestis chromosomal DNA replication elongation system. This invention also provides methods for the expression and purification of the anticipated five centrally important components of the P. aeruginosa and Y. pestis DNA polymerase III holoenzyme. This invention further provides a P. aeruginosa and Y. pestis minimal processive replicase. This invention also provides methods for the use of the fully reconstituted P. aeruginosa and Y. pestis replication system in a high-throughput screening format to identify compounds that are active as inhibitors of Pseudomonas and Yersinia DNA replication. Examples of such methods may be found in the Examples section.

### EXAMPLES

The following examples include a number of recombinant DNA and protein chemistry techniques known to those skilled in the art; see, for example, Sambrook et al., ibid. General Procedures (including starting cloning vectors)

### Example 1

### Construction of pA1-CB-NcoI Vector

To construct plasmid pA1-CB-NdeI, pA1-CB-NcoI was digested with NdeI. The overhanging ends were blunted with Klenow fragment to destroy the NdeI restriction site outside of the polylinker region. The linear plasmid was resealed forming pA1-CB-NcoI(NdeI-). This plasmid was transformed into DH5a and plasmids were isolated from one resulting ampicillin-resistant colony. The plasmids were screened for loss of the NdeI site. The region filled in by Klenow fragment was sequenced to confirm the loss of the NdeI site (ATG SEQ 661, primer P65-S2529). pA1-CB-NcoI(NdeI-) was digested with PacI and SpeI restriction enzymes. This removed the polylinker containing PacINcoI-spacer-KpnI-spacer-FseI-SpeI restriction sites. An annealed DNA duplex or adaptor/linker (shown below) containing PacI and SpeI sticky ends (ATG linker/adaptor P65-S1 and P65-A1) was inserted into the digested pA1-CB-NcoI(NdeI-) plasmid.

| | |
|---|---|
| 5′-TAACATATGAAAAAAAAAACCAGGTTGCTAG | (SEQ ID NO:134) |
| CGGTACCA-3′ | |
| 3′-TAATTGTATACTTTTTTTTTTGGTCCAACGA | (SEQ ID NO:135) |
| TCGCCATGGTGATC-5′ | |

The introduction of this adaptor/linker into pA1-CB-NcoI(NdeI-) formed a new polylinker containing the restriction sites PacI-NdeI-spacer-NheI-KpnI-FseI-SpeI. This plasmid was transformed into DH5a and the plasmids were isolated from one resulting ampicillin-resistant colony. These plasmids were screened for the introduction of the NdeI site. The region containing the inserted sequence was subjected to DNA sequencing to confirm insertion of the correct sequence (ATG SEQ #718, primer P38-S5576). This plasmid was named pA1-CB-NdeI and the positive isolate was grown and stored as a glycerol stock culture.

### Example 2

### Construction of pA1-CB-NdeI

To construct plasmid pA1-CB-NdeI, pA1-CB-NcoI was digested with NdeI. The overhanging ends were blunted with Kienow fragment to destroy the NdeI restriction site outside of the polylinker region. The linear plasmid was resealed forming pA1-CB-NcoI(NdeI-). This plasmid was transformed into DH5a and plasmids were isolated from one resulting ampicillin -resistant colony. The plasmids were screened for loss of a NdeI site. The region filled in by Klenow fragment was sequenced to confirm the loss of the NdeI site (ATG SEQ 661, primer P65-S2529). pA1-CB-NcoI(NdeI-) was digested with PacI and SpeI restriction enzymes. This removed the polylinker containing PacI/NcoI-spacer-KpnI-spacer-FseI-SpeI restriction sites. An annealed DNA duplex or adaptor/linker (shown in SEQ ID NO:134 and SEQ ID NO:135) containing PacI and SpeI sticky ends (ATG linker/adaptor P65-S1 and P65-A1) was inserted into the digested pA1-CB-NcoI(NdeI-) plasmid.

The introduction of this adaptor/linker into pA1-CB-NcoI(NdeI-) formed a new polylinker containing the restriction sites PacI-NdeI-spacer-NheI-KpnI-FseI-SpeI. This plasmid was transformed into DH5a and the plasmids were isolated from one resulting ampicillin-resistant colony. These plasmids were screened for the introduction of a NdeI site. The region containing the inserted sequence was subjected to DNA sequencing to confirm insertion of the correct sequence (ATG SEQ #718, primer P38-S5576). This plasmid was named pA1-CB-NdeI and the positive isolate was grown and stored as a glycerol stock culture.

### Example 3

### Identification of Subunits

P. aeruginosa subunits α #1, β, δ, δ′, and DnaX were identified using Ψ-BLAST or tblastn BLAST searches as described in copending U.S. patent application Ser. No. 09/906,179, filed Jul. 16, 2001).

To identify the genes encoding α #2, ε, χ, DnaG and SSB subunits from P. aeruginosa, databases were searched from sequencing projects underway at TIGR, The Institute for Genomic Research. The searches were initiated with α, 68 , χ, DnaG and SSB from E. coli, respectively. The searches were conducted using the tblastn BLAST search. The settings used in these tblastn BLAST (Gish, W. and States, D. J., Nature Genet. 3:266-272 (1993) This program compares a protein query sequence against a nucleotide database that has been translated in all six reading frames.

Y. pestis subunits α, β, δ, δ′, and DnaX were identified using tblastn BLAST searches as described in copending United States patent application Ser. No. 09/906,179, filed Jul. 16, 2001, incorporated by reference herein in its entirety. The genes encoding ε, θ, χ, ψ, DnaG and SSB subunits from Y. pestis were identified by searching sequencing projects underway at The Sanger Centre. The searches were conducted using the tblastn BLAST search as describe above. The searches were initiated with ε, θ, χ, ψ, DnaG and SSB from E. coli, respectively. Once the genes encoding the subunits were identified, the gene sequences were translated to protein sequences.

### Example 4

### General Procedures for Expressing and Analyzing Proteins

### 4.1. Transformation of Cells and Expression of Proteins

All intermediate plasmids are transformed into DH5α bacteria. Amp^{R} colonies are selected and the plasmids are screened for gain or loss of the appropriate restriction sites. The sequences of inserted DNA are confirmed by DNA sequencing. Ligations are performed in the presence of T4 DNA ligase and ATP. Protruding ends are blunted by treatment with the Klenow fragment of DNA polymerase I in the presence of Mg⁺⁺ and dNTPs.

Plasmids are transformed into MGC1030 E. coli bacteria (mcrA, mcrB, lamBDA(-), (RRND-RRNE)1, lexA3) and AP1.L1 E. coli. The parent to the AP1.L1 bacterial strain is Novagen BLR bacterial strain [F-, ompT hsdSB(rB- mB-) gal dcm.(srl-recA)306::Tn10]. A T1 phage-resistant version of this BLR strain was designated AP1.L1. Single colonies (at least 3 colonies from each transformation) of transformed cells selected for by ampicillin-resistance are inoculated into 2 ml of 2×YT culture media containing 100 μg/ml ampicillin and grown overnight at 37° C. in a shaking incubator. In the morning, 0.5 ml of the turbid culture from the overnight growth are inoculated into 1.5 ml of fresh 2×YT culture media. The cultures are grown for 1 hour at 37° C. with shaking and expression is induced by addition of isopropyl-β-D-thiogalactopyranoside (IPTG) to a final concentration of 1 mM. The cells are harvested by centrifugation 3 hours post-induction. The cell pellets are immediately resuspended in 1/10 culture volume of 2× Laemelli sample buffer (2× solution: 125 mM Tris-HCl (pH 6.8), 20% glycerol, 4% sodium dodecyl sulfate (SDS), 5% β-mercaptoethanol, and 0.005% bromophenol blue w/v), and sonicated to complete lysis of cells and to shear the DNA. The samples are heated for 10 minutes at 90-100° C., and centrifuged to remove insoluble debris. A small aliquot of each supernatant (3 μl) containing total cellular protein is electrophoresised onto a 4-20% gradient SDS-polyacrylamide mini-gel (Novex, EC60255; 1 mm thick, with 15 wells/gel) in 25 mM in Tris base, 192 mM glycine, and 0.1% SDS. The mini-gels are stained with Coomassie Blue. Proteins from induced cultures are compared with samples from non-induced cultures to ascertain the presence of target proteins only seen in induced cultures.

### 4.2. Expression of Tagged Proteins

If proteins are expressed as tagged proteins, d-biotin (10 μM) is included in the growth media. The total protein in each lysate is electrophoresised onto polyacrylamide gels as described above. The total protein in each lysate is then transferred (blotted) from polyacrylamide gel to nitrocellulose membrane using a Novex transfer apparatus at 30 V constant voltage in 12 mM Tris base, 96 mM glycine, 0.01% SDS (w/v), and 20% methanol (v/v) for 60 minutes at room temperature. The membrane is blocked in 0.2% Tween 20 (v/v)-TBS (TBST) (tris-buffered saline; 8 g/L NaCl, 0.2 g/L KCl, 3 g/L Tris-HCl (pH 7.4)) containing 5% non-fat dry milk (w/v) for 1 hour at room temperature. The blotted nitrocellulose is next rinsed with TBST, and then incubated in 2 μg/ml alkaline phosphatase-conjugated streptavidin (Pierce Chemical Co. #21324) in TBST for 1 hour at room temperature. Following extensive washing with TBST, the blots are developed with BCIP/NBT (KPL #50-81-07; one component system). The endogenous E. coli biotin-carboxyl carrier protein (biotin-CCP), ˜20 kDa is detectable in both induced and non-induced samples. Target proteins are observed as distinct bands in the induced cultures, but are not observed in the uninduced controls.

### 4.3. Verification of Protein Expression

Plasmids are transformed into AP1.L1 strain of E. coli. The parent to the AP1.L1 bacterial strain was Novagen BLR bacterial strain [F-, ompT hsdSB(rB- mB-) gal dcm δ(srl-recA)306::Tn10]. A T1 phage-resistant version of this BLR strain was designated AP1.L1. Single colonies (3 colonies form each transformation) of transformed cells selected for by ampicillin-resistance are inoculated into 2 ml of 2×YT culture media containing 100 μg/ml ampicillin and grown overnight at 37° C. in a shaking incubator. In the morning, 0.5 ml of the turbid culture from the overnight growth is inoculated into 1.5 ml of fresh 2×YT culture media. The cultures are grown for 1 hour at 37 ° C. with shaking and expression is induced by addition of isopropyl-β-D-thiogalactopyranoside (IPTG) to a final concentration of 1 mM. The cells are harvested by centrifugation 3 hours post-induction. The cell pellets are immediately resuspended in 1/10 culture volume of 2× Laemelli sample buffer (2× solution: 125 mM Tris-HCl (pH 6.8), 20% glycerol, 4% sodium dodecyl sulfate (SDS), 5% β-mercaptoethanol, and 0.005% bromophenol blue w/v), and sonicated to complete lysis of cells and to shear the DNA. The samples are heated for 10 minutes at 90-100 ° C., and centrifuged to remove insoluble debris. A small aliquot of each supernatant (3 μl) containing total cellular protein is analyzed by SDS-polyacrylamide electrophoresis on a 4-20% mini-gel (Novex, EC60255; 1 mm thick, with 15 wells/gel) in 25 mM in Tris base, 192 mM glycine, and 0.1% SDS. The mini-gels are stained with Coomassie Blue. Proteins bands migrating at sites expected for proteins with molecular masses corresponding to target subunits should be detected as distinct protein bands, but should not be observed in the uninduced control.

### 4.4. Large Scale Growth of Bacteria

Bacteria were grown in a 250-liter fermentor to produce cells for purification of Y. pestis proteins. F-medium was sterilized, glucose was added to 1% from a 40% sterile solution and ampicillin (100 mg/l) was added. A large-scale inoculum (28 l), was initiated from a 1 ml glycerol stock culture (i.e., culture stored in 15% glycerol at −80° C.) and grown overnight at 37 ° C. with 40 l/min aeration. The inoculum was transferred (approximately 4.2 l) to the 250-liter fermentor containing 180 l of F-medium with 1% glucose, and 100 mg/l ampicillin (starting OD₆₀₀ of 0.06). To calculate the amount of overnight culture to add to the fermentor, in this fermentation there was 180 l initial F-media, enough should be added to bring the media present in the fermentor to an OD₆₀₀=0.06. This allows enough time for the cell density to double 3-4 times before induction. The culture was incubated at 37° C., with 40 l/min aeration, and stirred at 20 rpm. Expression of Y. pestis proteins was induced by addition of IPTG to 1 mM when the culture reached an OD₆₀₀ of 0.79. Additional ampicillin (100 mg/l) was added at same time as induction. The temperature was maintained at approximately 37° C. throughout the growth. The pH was maintained at 7.2 throughout the growth by addition of NH₄OH. Cell harvest was initiated at various times (normally 3-4 hours) after induction at OD₆₀₀=approximately 6, and the cells were chilled to 10° C. during harvest. The harvest volume is normally between 150 and 200 L, and the final harvest weight is normally between 1.5 and 2.0 kg of cell paste. An equal amount (w/w) of 50 mM Tris (pH 7.5) and 10% sucrose solution is added to the cell paste. Cells are frozen by pouring the cells suspension into liquid nitrogen, and stored at −20° C., until processed. Quality control is carried out on cells in the inoculum and at harvest. Positive colonies are colonies grown from samples streaked on LB plates that also grow when the colony is transferred to LB plates containing a selective antibiotic.

### 4.5. Preparation of Fr I (Cell Lysis)

Lysis of cells is accomplished by creation of spheroplasts of the cells carrying the expressed Y. pestis proteins. Various amounts of a 1:1 suspension of frozen cells in Tris-sucrose, which had been stored at −20° C. is added to Tris-sucrose buffer that had been pre-warmed to 55° C. (2.75 ml/g of cells). To the stirred mixture, 0.5 M 1,4-dithiothreitol (DTT) (0.05 ml/g of cells) and lysis buffer (2M NaCl, 0.3M spermidine in Tris-sucrose adjusted to pH 7.5) (0.25 ml/g of cells) is added. Spermidine (18 mM) in the lysis buffer is added to keep the nucleoid condensed within partially disrupted cells and to displace DNA binding proteins. The pH of the slurry is adjusted to pH 7.5 by the addition of of 2 M Tris base, and lysozyme (Worthington Biochemical Corporation, cat #38H2088) is added resuspended in 5 ml of Tris-sucrose buffer (4 mg lysozyme/g of cells). The slurry is placed in a 250 ml centrifuge bottles after stirring 5 min and incubated at 4° C. for 1 hour. The 250 ml centrifuge bottle are then placed in a 37° C. swirling water bath and gently inverted every 30 seconds for 4 minutes. The insoluble cellular components are removed by centrifugation (23,000×g, 60 min, 4° C.). The recovered supernatant (typically approximately 100 ml) constitutes Fraction I (Fr I).

### 4.6. Reconstitution Assays

The template in reconstitution assays is RNA primed M13Gori single stranded circular DNA. RNA primed M13Gori single-stranded DNA was prepared (9.5 ml) by adding: 0.5 ml MgOAc (250 mM), 1.125 ml M13Gori (240 μM, nt), 0.2 ml purified E. coli SSB proteins (4.3 mg/ml), 1.5 ml dNTP mix (400 μM dATP, dCTP, dGTP and 150 μM [³H]-dTTP; ca. 100 cpm/pmol of total nucleotide incorporated by polymerase (sp. act dTTP/4)), 0.5 ml rNTP mix (5 mM of each ATP, CTP, GTP and UTP), 0.025 ml purified E. coli primase (0.665 mg/ml) and 5.65 ml EDB (50 mM HEPES (pH 7.5), 20% glycerol, 0.02 % NP40, 0.2 mg/ml BSA). The radioactive dNTP mix was not used in the priming reaction but was used by the replication polymerase when it is added in the actual reconstitution reaction. The priming mix was incubated at 30° C. for 5 min and then placed on ice. The mixture was divided into 400 μl aliquots and stored at −80° C. until used. This mixture was used in all reconstitution assays and is referred to as the primed-template mix. The RNA primed template mixtures were used in all M13Gori assays and are referred to as the primed template mix. The Y. pestis (or E. coli) components (core, clamp-loading complex, and β) are diluted to the desired concentration in EDB buffer in a total volume of 6 μl and combined with 19 μl of the primed template mix to yield a 25 μl reaction. Following mixing, the reaction contents were incubated for 5 min at 30° C. The reaction is terminated by placing the reaction tube on ice and adding 2 drops of 0.2 M sodium pyrophosphate and 0.5 ml 10% TCA. The solution is filtered under vacuum through Whatman GF/C glass microfiber filters. The filters are then washed with 3 assay tube volumes (3×5 ml) of 1 M HCl/0.2 M sodium pyrophosphate and 1 assay tube volume (5 ml) of 95% EtOH and dried using a heat lamp. The amount of radiolabeled nucleotides incorporated is quantified by scintillation counting. One unit of activity is 1 pmol of total deoxyribonucleotide incorporated per min at 60° C.

### Examples Relating to Yerisinia pestis Nucleic Acids and Proteins

Pol III Core

The goal was to make a 3-gene operon containing the pol III core subunits (ε, θ and α). A step-wise procedure was utilized, starting with each gene individually cloned into a vector so that it can be expressed alone followed by a step-wise combination of the genes into a 3-gene operon.

### Example 5

### Molecular Cloning of Y. pestis dnaE, the Gene Encoding the Replicative Polymerase dnaE (alpha)

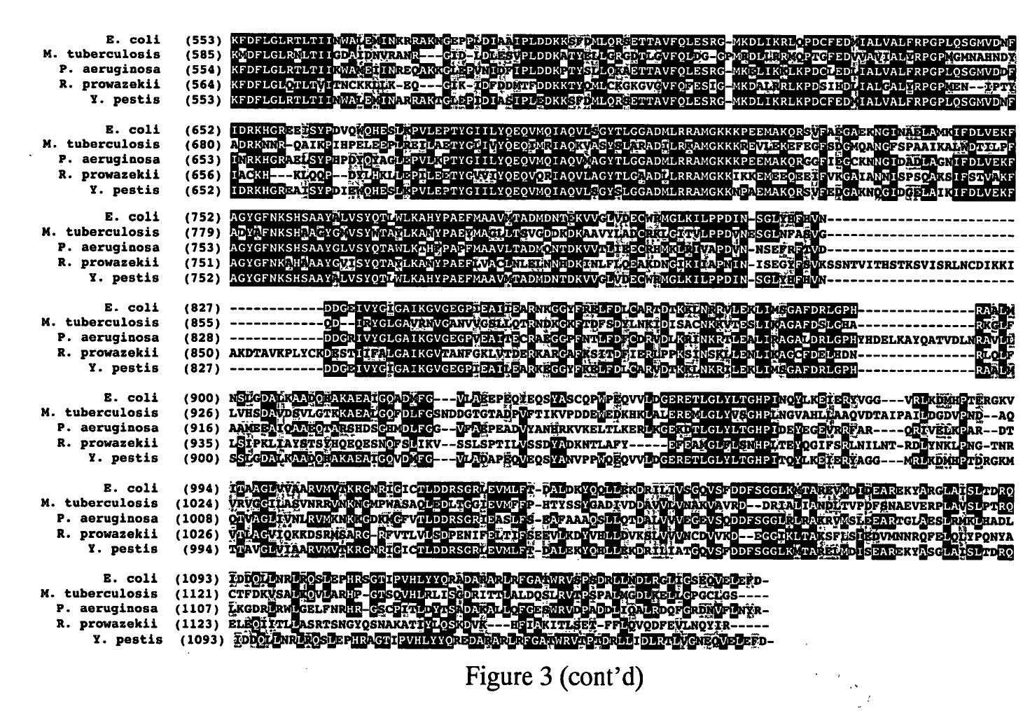

The recently completed Y. pestis genome database (Sanger Database (2001)) was searched for genes that encode the DNA polymerase III holoenzyme subunits. The gene for dnaE (α) subunit was identified by its homology to other known DnaE (α) subunits. Comparison of amino acid sequences for DnaE proteins from Y. pestis and a selected subset of bacteria uniformly used for comparison throughout this application, shows that they are homologous and align over the entire length of the protein sequences (FIG. 3).

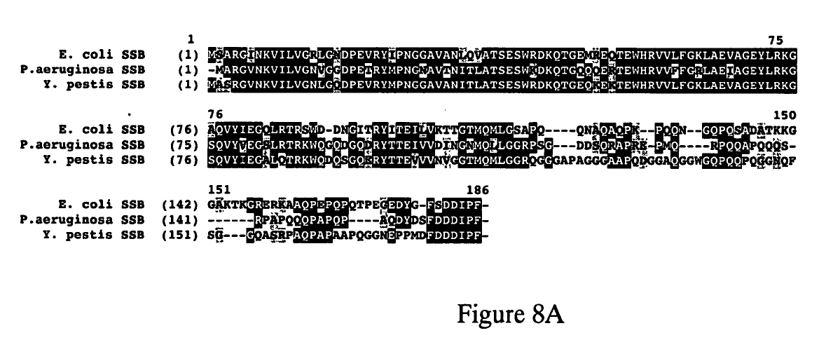

In this example, PCR amplification of the dnaE gene (α subunit) out of the genomic DNA is performed. The PCR product was inserted into the vector pA1-CB-NdeI. The polyclonal region of this vector is shown in FIG. 10. The α subunit is composed of 1161 amino acids encoded by 3483 nucleotides of the dnaE gene. The nucleotide sequence of dnaE is represented by SEQ ID NO:1. Codons 2 and 4 (shown in the nucleotide sequence in underlined letters) which code for Ala and Pro are low usage codons in E. coli and are changed to high usage codons in the PCR reaction. The change is from ATGGCCGAACCT (SEQ ID NO:4) to ATGGCTGAACCG (SEQ ID NO:5). The amino acid sequence of alpha is represented by SEQ ID NO:3.

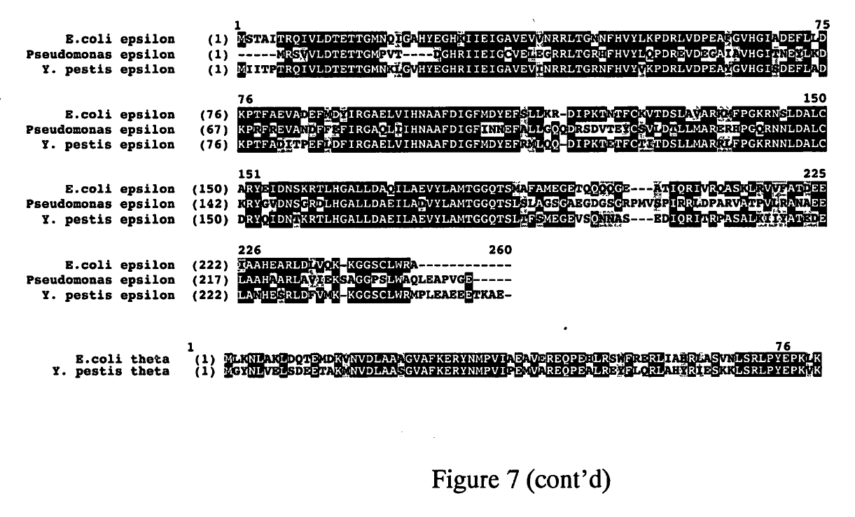

Because this is a large gene, it was amplified by PCR it in two different reactions. In the first PCR reaction the 5′ 1445 nucleotides are amplified. There is a unique NheI restriction site located at nucleotide position 1440-1445 in the dnaE gene. This region of the dnaE gene is shown in FIG. 9. The forward/sense primer for the first PCR reaction is: 5′-GATCttaattaacctaggATGGCTGAACCGCGTTTTGTCCACCTGCGT-3′ (48-mer) (SEQ ID NO:7). The 5′ clamp region (GATC) is followed by a PacI restriction site (underlined italic letters) for insertion into pA1-CB-NdeI, followed by an AvrII restriction site (italic letters) for use in the later construction of the core operon. Next, there are the 12 nts (first four codons, with mutations in codons 2 and 4), shown in capital italic letters. Finally, there are the 18 nucleotides complementary to the 5′ end of dnaE beginning at codon 5 (underlined capital letters).

The reverse/antisense primer is complementary to a region downstream of the unique NheI restriction site. This primer is: 5′-CTGACCTCTTCATCAGCTT-3′ (19-mer) (SEQ ID NO:1 17). There is also a unique KpnI site just upstream of the unique NheI that could be used, and also a unique PstI site about 800 bases downstream. The NheI restriction site is shown in boldface letters and the sequence of the selected primers upstream (forward second PCR reaction) and downstream (reverse first PCR reaction) of the unique NheI are shown in boldface underlined letters.

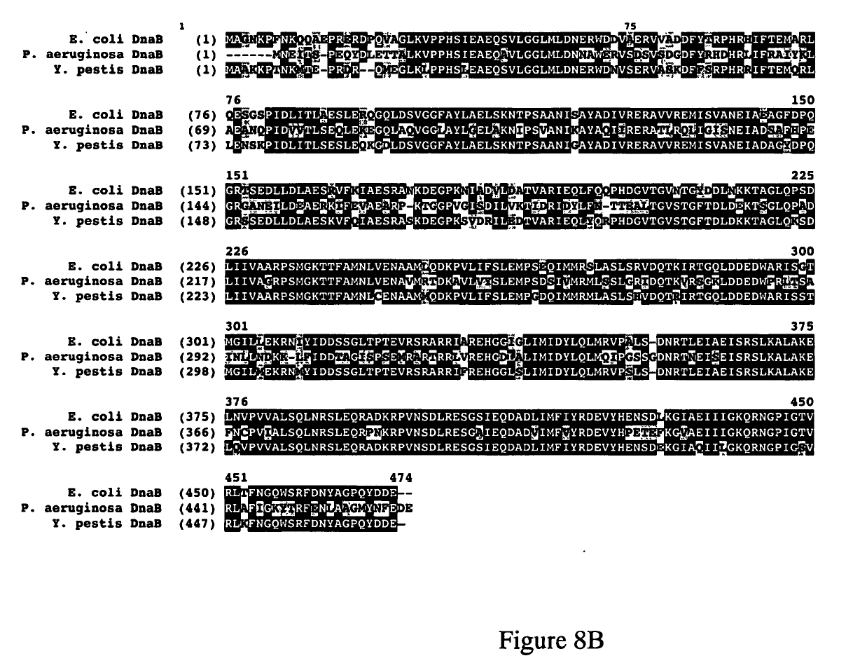

Both the PCR product and the plasmid pA1-CB-NdeI were digested with PacI/NheI restriction enzymes. The PCR fragment containing the 5′ portion of the dnaE gene was inserted into the PacI/NheI digested pA1-CB-NdeI. This resulted in plasmid pA1-YP-dnaE5′, which contained the 5′ portion of the dnaE gene that is spaced 14 nucleotides downstream of the ribosome binding site (RBS) in pA1-CB-NdeI, which is a little above the optimal spacing distance. However, when placed in the core operon the spacing is optimal. A schematic depiction of pA1-YP-dnaE5′ is shown in FIG. 11. In the second PCR reaction, the 3′ 2038 nucleotides of the dnaE gene were amplified. The forward/sense primer located upstream of the unique NheI restriction site is: 5′-CTGGAAAAAGCGTTTGCAG-3′ (19-mer) (SEQ ID NO:6). The reverse/antisense primer is: 5′-GATCactagtttaTTAGTCAAATTCCAGTTCC-3′ (32-mer) (SEQ ID NO:118). The 5′ clamp region (GATC) is followed by a SpeI restriction site (italic letters) for insertion into pA1-YP-dnaE5′ (this site will also be used in construction of the core operon) and an additional stop codon (double underlined letters). Finally, there are 19 nucleotides (including the native stop codon) that are complementary to the 3′ end of the dnaE gene.

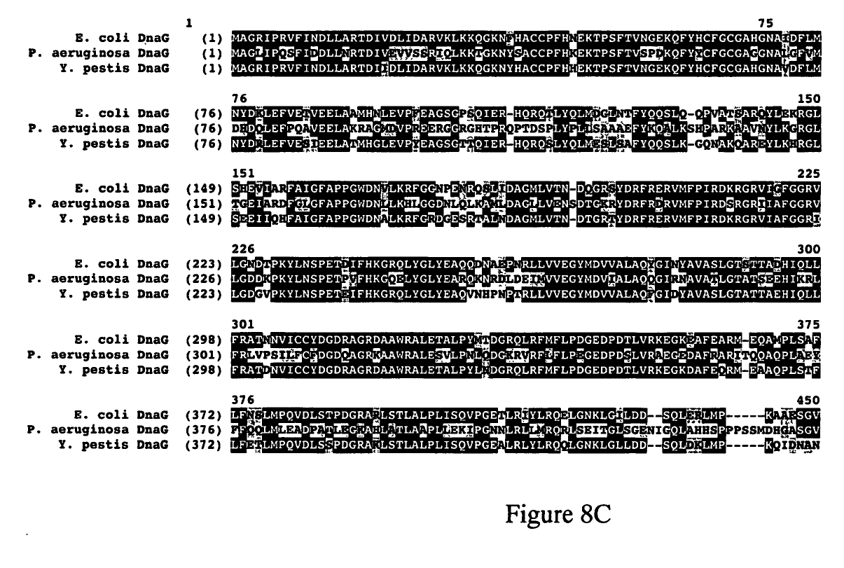

Both the PCR product and the plasmid pA1-YP-dnaE5′ were digested with NheI/SpeI restriction enzymes. The PCR fragment containing the 3′ portion of the dnaE gene were inserted into the NheI/SpeI digested pA1-YP-dnaE5′. This resulted in plasmid pA1-YP-dnaE, which contains the entire dnaE gene that is spaced 14 nucleotides downstream of the RBS in pA1-CB-NdeI, which is somewhat longer than the optimal spacing distance. However, when placed in the core operon, the spacing is optimal. Sequencing of the dnaE insert has been completed confirming the presence of the native (unmutated) dnaE gene in the vector as outlined. A schematic depiction of pA1-YP-dnaE vector is shown in FIG. 12.

### Example 6

### Expression of Alpha in E. coli

Purification for Y. pestis DnaE expressed by itself is accomplished with reference to previous work. Enzyme recovery and location can be monitored using an art-known simple assay that does not require auxiliary factors—the nonprocessive filling of gaps in nuclease-activated DNA (Kim, D. R. and McHenry, C. S. (1996) J Biol Chem 271, 20681-2068979). Generally, DNA polymerase III a subunits are relatively insoluble in ammonium sulfate. The lowest concentration of ammonium sulfate that precipitates at least 90% of Y. pestis DnaE was used. The majority of E. coli proteins is left in the supernatant. Cation exchange chromatography generally gives good purification of polymerases. They bind the resin under conditions where most E. coli proteins flow through, the polymeraseis eluted with high purification in a salt gradient. Obtaining pure protein at this stage is anticipated, but if it is not achieved, additional art-known chromatographic steps will be developed that achieve maximal purification with preservation of activity. Total protein levels will be determined by the Bradford procedure (Bradford, ibid.) in this and other purifications described in this application.

### Example 7

### Molecular Cloning of Y. pestis dnaQ, the Gene Encoding the Epsilon Subunit

The dnaQ gene (ε subunit) was amplified out of the genomic DNA by PCR and inserted into the vector pA1-CB-NdeI (the polyclonal region of this vector is shown in FIG. 10). The ε subunit is composed of 255 amino acids encoded by 765 nucleotides of the dnaQ gene. The nucleotide sequence of dnaQ is represented by SEQ ID NO:8. The amino acid sequence of ε subunit is represented by SEQ ID NO:10.

The forward/sense primer is: 5′-GATCcatATGATCATTACACCGAC-3′ (24-mer) (SEQ ID NO:11). The 5′ clamp region (GATC) is followed by a NdeI site for insertion into pA1-CB-NdeI (italic letters). The NdeI restriction site overlaps the ATG start codon on dnaQ. The 17 nucleotides corresponding to the 5′ end of dnaQ are shown in underlined capital letters.

The reverse/antisense primer is:

| 5′-GATCggtacctgtctgcagattcc | (49-mer) | (SEQ ID NO:12) |
|---|---|---|
| tcctttaTTATTCTGCTTTTGTCTC- | | |
| 3′. | | |

The 5′ clamp region (GATC) is followed by a KpnI restriction site (italic letters), for insertion into pA1-CB-NdeI, and is separated from a PstI site (italic letters), which will be used in the construction of the core operon, by a 3-base spacer (boldface tgt). This 3-base spacer is needed to allow for efficient restriction digestion by both KpnI and PstI in a later step for constructing the core operon. Following the restriction sites is another 3-base spacer (bold underline letters) to optimize the spacing between the RBS and a downstream gene in the construction of the core operon described below. Following this spacer is a RBS (boldface letters) for the downstream gene that will be added later in construction of the core operon and an additional stop codon (double underline letters). Finally, there are the 18 nucleotides complementary to the 3′ end of the dnaQ gene, which includes the native stop codon (underlined capital letters).

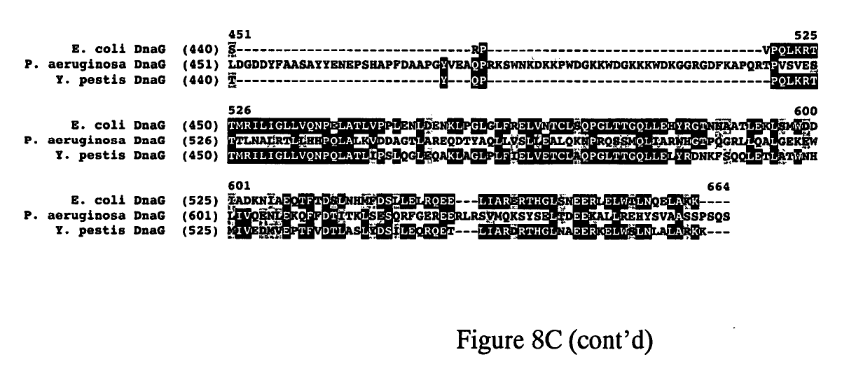

Both the dnaQ PCR product and the plasmid pA1-CB-NdeI were digested with NdeI/KpnI restriction enzymes. The fragment of the PCR product containing the dnaQ gene was inserted into the NdeI/KpnI digested pA1-CB-NdeI. This placed the dnaQ gene optimally spaced from the upstream RBS and resulted in the plasmid pA1-YP-dnaQ, which is schematically shown in FIG. 13.

### Example 8

### Molecular Cloning of Y. pestis holE, the Gene Encoding the Theta Subunit

This example describes PCR amplification of the holE gene (θ) out of the genomic DNA and inserting it into the vector pA1-CB-NdeI. θ subunit is composed of 77 amino acids encoded by 231 nucleotides of the holE gene. The nucleotide sequence of holE is represented by SEQ ID NO:13. Codons 2, 3 and 4 which code for Gly, Tyr and Asn are low usage codons in E. coli and were changed to high usage codons in the PCR reaction. The change will be from GGATATAAT to GGCTACAAC. The amino acid sequence of θ is represented by SEQ ID NO:15.

The forward/sense primer is: 5′-GATCttaattaactgcagATGGGCTACAACTTGGTAGAGCTTTCTGACG-3′ (49-mer) (SEQ ID NO:16). The 5′ clamp region (GATC) is followed by a PacI restriction site (underlined italic letters) for insertion into pA1-CB-NdeI, followed by a PstI restriction site (italic letters) for use later in core operon construction. This is followed by 12 nucleotides (four codons) that include the start ATG and mutated codons 2, 3 and 4 (shown as capitalized italic letters). Finally, the 19 nucleotides (capital letters and underlined) that correspond to the 5′ nucleotides beginning at codon 5 of holE.

The reverse/antisense primer is:

| | | |
|---|---|---|
| 5′-GATCggtacccctaggattcctcc | (46-mer) | (SEQ ID NO:17) |
| tttaTTACTTCACTTTAGGTTC-3′. | | |

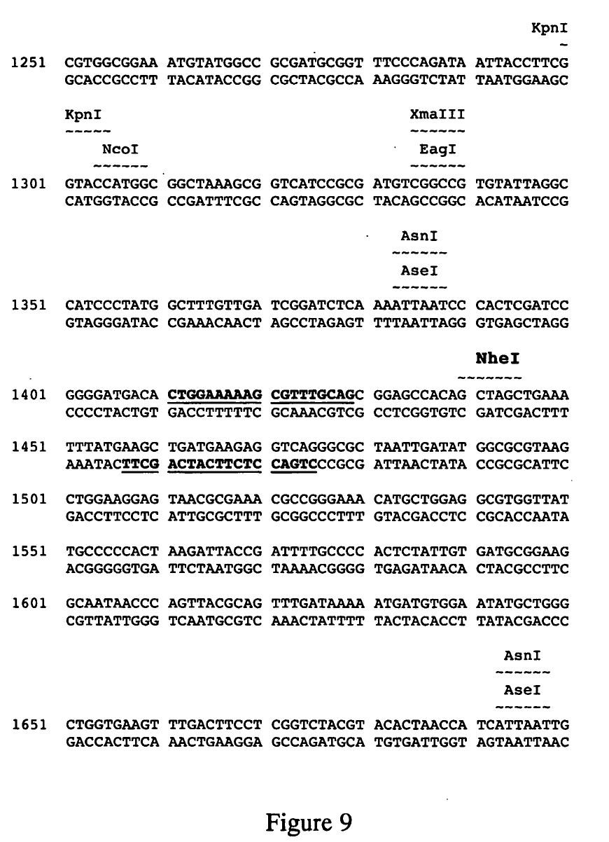

The 5′ clamp region (GATC) is followed by a KpnI restriction site (italic letters) for insertion into pA1-CB-NdeI, followed by an AvrII restriction site (underlined italic letters) for later construction of the core operon. A 3-base spacer (boldface att) for optimal spacing of the downstream gene (in later construction of the core operon) follows the restriction sites. This is followed by the RBS (bold underlined letters) and an additional stop codon (double underlined letters). Finally, 18 nucleotides complementary to the 3′ end of the holE gene are shown in underlined capital letters. Both the PCR product and the plasmid pA1-CB-NdeI were digested with PacI/KpnI restriction enzymes. The PCR fragment containing the holE gene was inserted into the PacIKpnI digested pA1-CB-NdeI. This resulted in plasmid pA1-YP-holE, which contains the holE gene that is spaced 14 nucleotides downstream of the RBS in pA1-CB-NdeI, which is somewhat longer than the optimal spacing distance. However, when placed in the core operon, the spacing is optimal. A schematic depiction of pA1-YP-holE is shown in FIG. 14.

### Example 9

### Operon Construction

### 9.1. Construction of HolQE operon

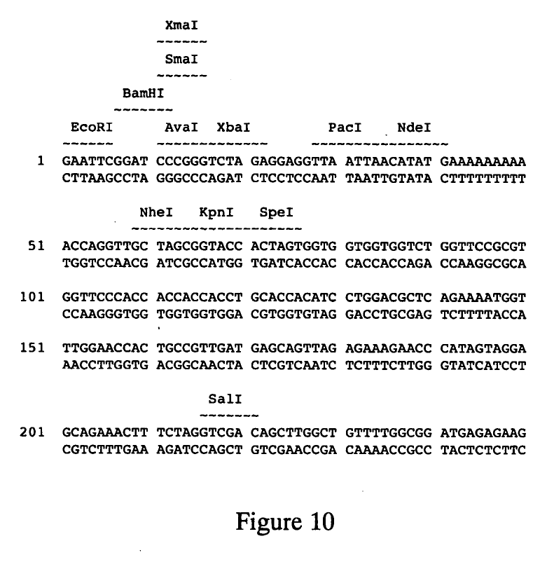

In this example, a 2-gene operon that contains dnaQ and holE genes was constructed. This was accomplished by digesting both pA1-YP-dnaQ and pA1-YP-holE with PstI/KpnI. The small fragment from pA1-YP-holE that contains the holE gene between the PstI/KpnI sites was inserted into the digested pA1-YP-dnaQ plasmid. This placed holE downstream of dnaQ and also downstream with optimal spacing from the RBS created in pA1-YP-dnaQ. This plasmid is referred to as pA1-YP-QE and is schematically shown in FIG. 15.

### 9.2. Construction of Core Operon

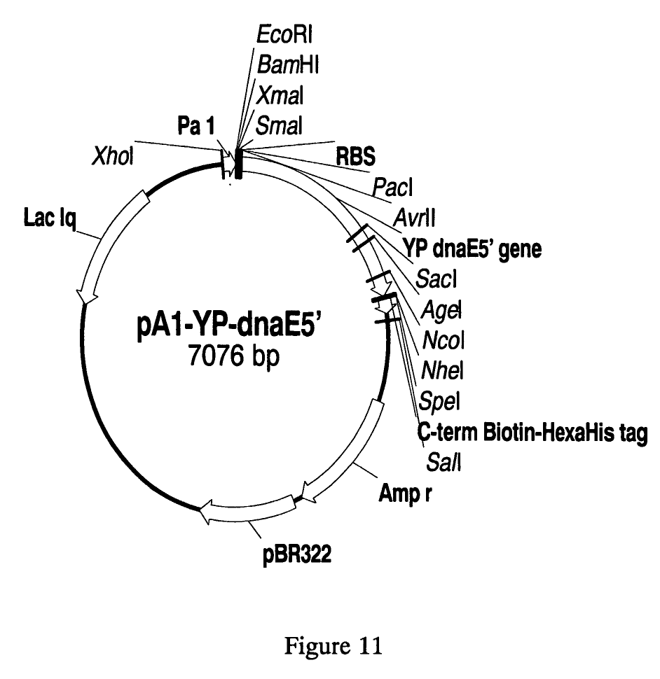

This example describes the 3-gene (core) operon that contains dnaQ, hole and dnaE. This was accomplished by digesting both pA1-YP-QE and pA1-YP-dnaE with AvrII/SpeI. The small fragment from pA1-YP-dnaE that contains the dnaE gene (a subunit) between the AvrII/SpeI sites was inserted into the digested pA1-YP-dnaQE plasmid. This placed dnaE downstream of dnaQ-holE and also downstream with optimal spacing from the RBS created in pA1-YP-holE. This plasmid was named pA1-YP-core and is depicted schematically in FIG. 16.

### 9.3. Expression of Core Operon

pA1-YP-core was transformed into AP1.L1 strain of E. coli and expressed as described in EXAMPLE 4.1. Expressed proteins were analyzed by SDS-PAGE as described in EXAMPLE 4.1. The mini-gels were stained with Coomassie Blue. Proteins bands migrating at sites expected for proteins with molecular masses corresponding to a (130 kDa), ε (28.9 kDa), and θ (9 kDa) subunits could be detected as distinct protein bands, but were not observed in the uninduced control.

### Example 10

### Optimization of expression of Y. pestis core proteins in E. coli

In an attempt to optimize the yield of expressed recombinant Y. pestis core proteins, induction times were analyzed for pA1-YP-core. F-media (Bacto Yeast Extract, 14 g/l; Bacto Tryptone, 8 g/l; potassium phosphate-dibasic, 12 g/l; potassium phosphate-monobasic, 1.2 g/l; pH 7.2, 1% glucose) were used as a growth medium. A small amount of F-media (10-20 ml) containing ampicillin was inoculated with the target bacteria and grown overnight at 37° C. while shaking. This overnight growth was used to inoculate fresh F-media containing ampicillin pre-warmed to 37 ° C. The fresh media was inoculated at a 20:1 ratio using the culture grown overnight. This allowed enough time for cell density to double 3-4 times before induction. The freshly inoculated cultures were grown to an OD₆₀₀=0.6-0.8 and expression was induced by addition of IPTG to 1 mM.

Equal sample volumes (5 ml) of cultures were collected at the time of induction and every hour after induction up to 5 hours post induction for analysis to determine optimum growth times. The OD₆₀₀ for each sample was determined. The samples collected were centrifuged in a Fisher Centrific Model 228 (1380×g) for 10 min. The supernatant was discarded and the cell pellets were retained for analysis. To maintain equal concentration of total protein in each sample, 50 μl of Laemmli lysis buffer (125 mM Tris-HCl, (pH 6.8), 20% glycerol, 5% SDS) was added per OD₆₀₀ unit of each sample multiplied by the sample volumes (5 ml). The cell pellets were resuspended and heated to 90-100° C. for 10 min. The samples were centrifuged at maximum rpm (16,000×g) for 10 min using a tabletop microfuge, and the supernatant was retained. Small aliquots containing total cellular protein, of each supernatant (5 μl) were loaded onto a 10% or a 5-20% gradient SDS-polyacrylamide gel (16×18×0.75 cm) in 25 mM in Tris base, 192 mM glycine, and 0.1% SDS. The gels were run for 2 h at 250 volts and the proteins were visualized with Coomassie Blue staining.

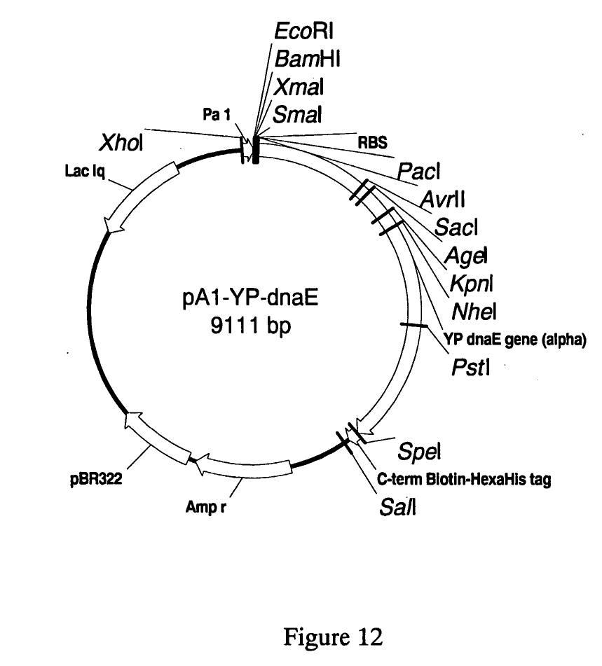

The results of the growth optimization analyzed by SDS-polyacrylamide gel electrophoresis for pA1-YP-core is shown in FIG. 17. In this gel, Y. pestis a expresses to high levels, approaching about 10% of the total cellular protein. Molecular mass markers are shown on the right side of the gel.

### Example 11

### Large Scale Growth of Core Operon

The large-scale growth of E. coli containing the pA1-YP-core plasmid was as described in Example 4.4, however, the bacteria were allowed to continue growing for 3.5 h post-induction giving a harvest weight of 1.44 kg. Quality control results showed 10 out of 10 positive colonies on ampicillin-containing medium in the inoculum and 10 out of 10 positive colonies on ampicillin-containing medium at harvest.

### Example 12

### Purification of Y. pestis Core

As an initial purification step, many endogenous E. coli proteins can be removed by adding ammonium sulfate (AS) to concentrations that cause the protein of interest (and some endogenous proteins) to precipitate out of solution, while other proteins remain in solution. The precipitated protein can then be separated from the proteins still in solution by centrifugation. Each protein precipitates out of solution at different concentrations of ammonium sulfate (depending on amino acid composition, distribution of polar/non-polar surface exposed amino acids, molecular shape and level of hydration). Therefore, the concentration of ammonium sulfate (expressed as percent saturation) in which each target protein precipitates out of solution has to be determined.

70 g of a 1:1 suspension of frozen cells (35 g cells) in Tris-sucrose, which had been stored at −20° C. were lysed to determine the optimum concentration of ammonium sulfate with which to precipitate the largest amount of the core complex containing maximum activity but retaining the smallest amount of contaminating protein.

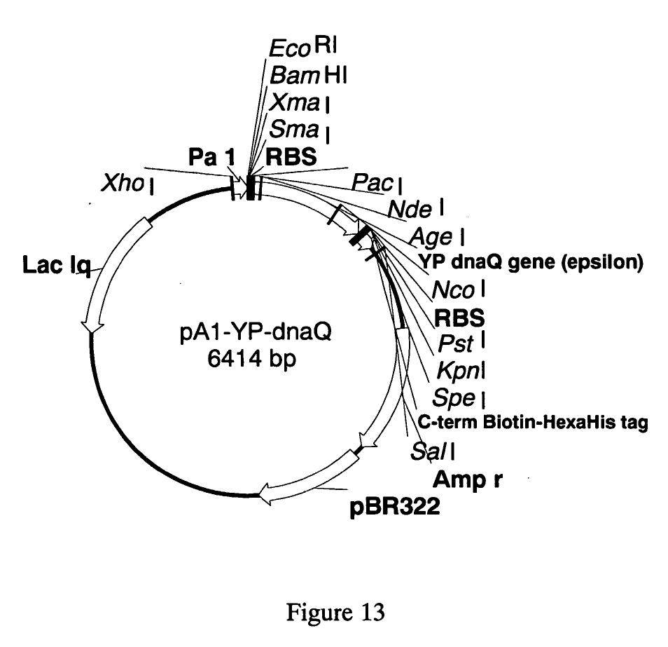

Fr I was divided into seven samples of 15 ml each and labeled 25%, 30%, 35%, 40%, 45%, 50% and 60%. The protein in each sample was precipitated by adding varying amounts ammonium sulfate so that the final concentration of ammonium sulfate was: 25%, 30%, 35%, 40%, 45%, 50%, and 60% saturation, respectively, at 4° C. The mixture was stirred for an additional 30 min at 4° C. and the precipitate was collected by centrifugation (23,000×g, 45 min, 0° C.). The supernatant was removed from each sample and the resulting pellets were resuspended in buffer (25 mM Hepes (pH 7.5), 5 mM EDTA, 10% glycerol, 5 mM DTT, 100 mM NaCl) resulting in a Fr II for each (7) ammonium sulfated precipitated samples. Protein concentrations of samples from both the supernatant and the different Fr II's are shown in FIG. 18. This was confirmed by SDS-polyacrylamide gel analysis of the resuspended protein pellets (data not shown).

To purify Y. pestis core 400 g of cells containing Y. pestis core was lysed to form Fr I as described above and resulted in 1270 ml of Fr I lysate. The protein in the sample was precipitated by gradually adding ammonium sulfate so that the final concentration of ammonium sulfate was 45% saturation at 4° C. The mixture was stirred for an additional 30 min at 4° C. and the precipitate was collected by centrifugation (23,000×g, 45 min, 0° C.). The protein pellet was resuspended in TG.51 buffer and diluted (2100 ml, Fr II) to the conductivity of a DEAE chromatography column (300 ml, 5 cm×15 cm) equilibrated in TG.51/25 mM NaCl buffer. The sample was loaded onto the DEAE column, washed with 3 column volumes of TG.51/25 mM NaCl buffer and eluted with 10 column volumes of TG.51 buffer containing a 25-400 mM NaCl gradient. Fractions (150) were collected containing 25 ml each. The protein eluted in one broad peak between fractions 40-140. The activity was contained in a single peak encompassed within the broad protein peak in fractions 96-111 (400 ml). 100% of the activity was recovered within this peak. SDS-polyacrylamide gel analysis indicated that the activity peak contained Y. pestis core, but the core complex only made up approximately 20% of the total protein. The protein in the sample was precipitated by ammonium sulfate so that the final concentration of ammonium sulfate was 70% saturation at 4° C. The mixture was stirred for an additional 30 min at 4° C. and the precipitate was collected by centrifugation (23,000×g, 45 min, 0° C.), quick frozen in liquid nitrogen and stored at −80° C.

The protein pellets containing Y. pestis core were resuspended in 200 ml imidazole buffer (50 mM imidazole, (pH 6.8), 10% glycerol, 50 mM NaCl, 5.0 mM DTT) and homogenized using a Dounce homogenizer. The sample was clarified by centrifugation (16,000×g) and the resulting sample constituted Fr III. The sample was diluted (430 ml) using Imidazole buffer minus NaCl to the conductivity of a hydroxylapatite chromatography column (200 ml, 5 cm×10 cm) equilibrated in imidazole buffer. The sample was loaded onto the hydroxylapatite column, washed with 3 column volumes of imidazole buffer/plus 10 mM KPO₄ and eluted with 10 column volumes of Imidazole buffer containing a 10-150 KPO₄ gradient. Fractions (80) were collected containing 25 ml each. The protein eluted in a peak between fractions 15 and 50. The activity eluted in a single peak within the broader protein peak. Fractions 25-37 were pooled (325 ml) and contained 100% of the total activity loaded onto the column. SDS-polyacrylamide gel electrophoresis indicated that the proteins of Y. pestis core complex made up approximately 50% of the total protein in the pool. The protein in the sample was precipitated by ammonium sulfate so that the final concentration of ammonium sulfate was 50% saturation at 4 ° C. The mixture was stirred for an additional 30 min at 4° C. and the precipitate was collected by centrifugation (23,000×g, 45 min, 0° C.), quick frozen in liquid nitrogen and stored at −80° C.

The protein pellets containing Y. pestis core from the hydroxylapatite column were resuspended in 100 ml imidazole buffer plus 1 mM EDTA and homogenized using a Dounce homogenizer. The sample was clarified by centrifugation (16,000×g) and the resulting sample constituted Fr IV. The sample was diluted (280 ml) using imidazole buffer minus NaCl to the conductivity of a P-11 phosphocellulose (Whatman) chromatography column (200 ml, 5 cm×10 cm) equilibrated in Imidazole buffer plus 1 mM EDTA. The sample was loaded onto the P-11 phosphocellulose column, washed with 4 column volumes of imidazole buffer plus 1 mM EDTA and eluted with 10 column volumes of imidazole buffer containing a 25-300 mM NaCl gradient. Fractions (80) were collected containing 25 ml each. The protein eluted in a peak between fractions 10 and 30. The activity eluted in a peak defined by the protein peak. Fractions 11-22 were pooled (300 ml) and contained 75% of the total activity loaded onto the column. SDS-polyacrylamide gel electrophoresis analysis of the fractions indicated that Y. pestis core was greater than 90% homogeneous. The protein in the sample was precipitated by ammonium sulfate so that the final concentration of ammonium sulfate was 50% saturation at 4° C. The mixture was stirred for an additional 30 min at 4° C. and the precipitate was collected by centrifugation (23,000×g, 45 min, 0° C.), quick frozen in liquid nitrogen and stored at −80° C.

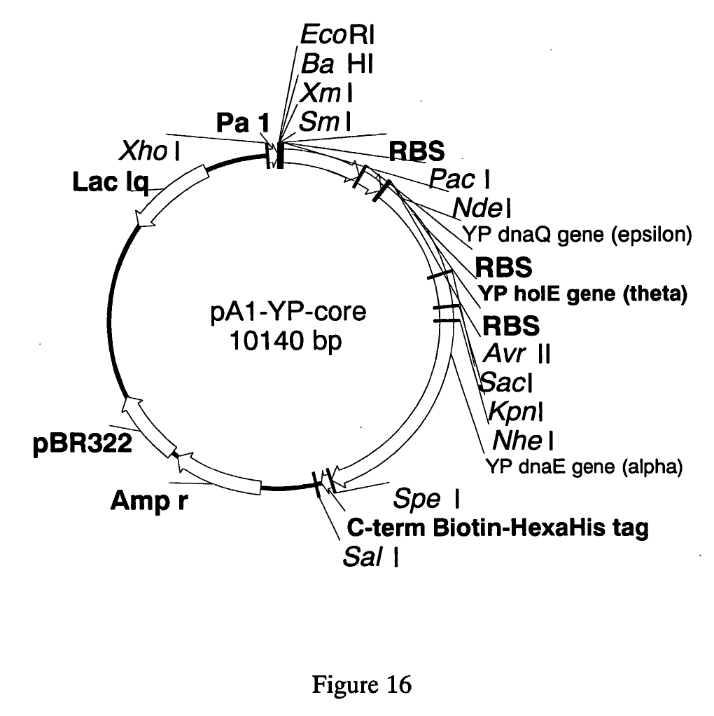

The protein pellets containing Y. pestis core was resuspended in 6 ml of HG.05 buffer (25 mM HEPES, (pH 7.5), 50 mM KCl, 10% glycerol, 5 mM DTT and 1 mM EDTA) and homogenized using a Dounce homogenizer. The sample was clarified by centrifugation (16,000×g) and the resulting sample was Fr V. The sample was loaded onto a Sephacryl® S-200 (Amersham Pharmacia Biotech) chromatography column (250 ml, 1:20 diameter:heigth ratio) equilibrated with HG.05 buffer. The procedure included running the buffer above the resin down to the resin bed, gently pipetted the sample onto the resin, pumping the sample into the resin, then 2 ml of buffer was gently added onto the top of the resin and pumped into the resin. The running buffer was then added to the top of the resin and elution was continued. The protein was eluted at a flow rate of 0.22 ml/min and 3 ml fractions were collected. Fractions 35-38 (12 ml) contained all of the loaded activity and were pooled (Fr VI). Fr VI was aliquoted and stored at −80° C. and represents the stock supply of Y. pestis core. At each step of the purification process the purity of Y. pestis core was analyzed by SDS polyacrylamide gel electrophoresis. A summary gel of the pooled protein for each purification step is shown in FIG. 21). Densitometry scan of the protein bands in Fr VI indicate that the ratio of α, ε, and θ in the core complex is 1:1:1, respectively. The protein concentration and activity was also characterized at each purification step (Table II).

TABLE II

| | | | Protein | | Specific |
|---|---|---|---|---|---|
| | Volume | | concentration | Activity | Activity |
| Fraction | (ml) | Protein (mg) | (mg/ml) | (Units) | (Units/mg) |
| Fr I | 1270 | 22000 | 17.3 | — | — |
| Fr II | 2100 | 5440 | 2.6 | 1.3 × 10⁹ | 2.4 × 10⁵ |
| Fr III | 430 | 900 | 2.1 | 2.0 × 10⁹ | 2.2 × 10⁶ |
| Fr IV | 280 | 370 | 1.3 | 8.6 × 10⁸ | 2.3 × 10⁶ |
| Fr V | 6 | 57 | 9.5 | 6.0 × 10⁸ | 1.1 × 10⁷ |
| Fr VI | 12 | 51 | 4.3 | 5.8 × 10⁸ | 1.1 × 10⁷ |

### Example 13

### Characterization of Gap-Filling Activity of Y. pestis Core and Determination of Optimal Ammonium Sulfate Conentration for Precipitation of Y. pestis Core

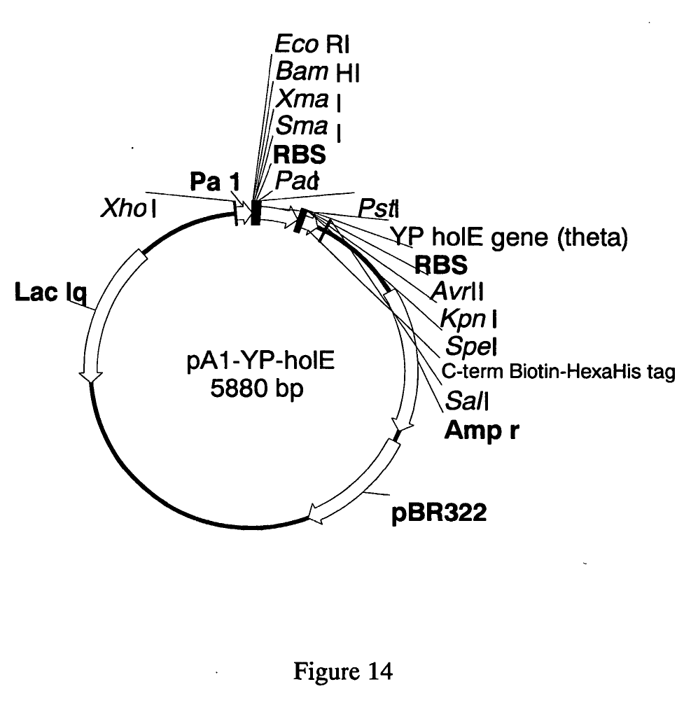

The activity of Y. pestis core was initially assayed using gap-filling assays. A series of reactions containing titration of each Fr II was preformed (FIG. 19). The reactions (25 μl) were initiated by addition of 2 μl of various dilutions of Fr II to a solution containing dNTPs (100 cpm [³H]/pmol dNTPs), 10 mM MgCl₂ and 5 μg of activated calf thymus DNA. The DNA was prepared as previously described (McHenry, C. S. and Crow, W. (1979) J Biol Chem 254, 1748-1753). The reaction was incubated for 5 min at 30° C. One unit is defined as the amount of enzyme required to incorporate 1 pmol of dNTPs per min at 30° C.

### Example 14

### Reconstitution of Core with E. coli Beta and Tau Complex

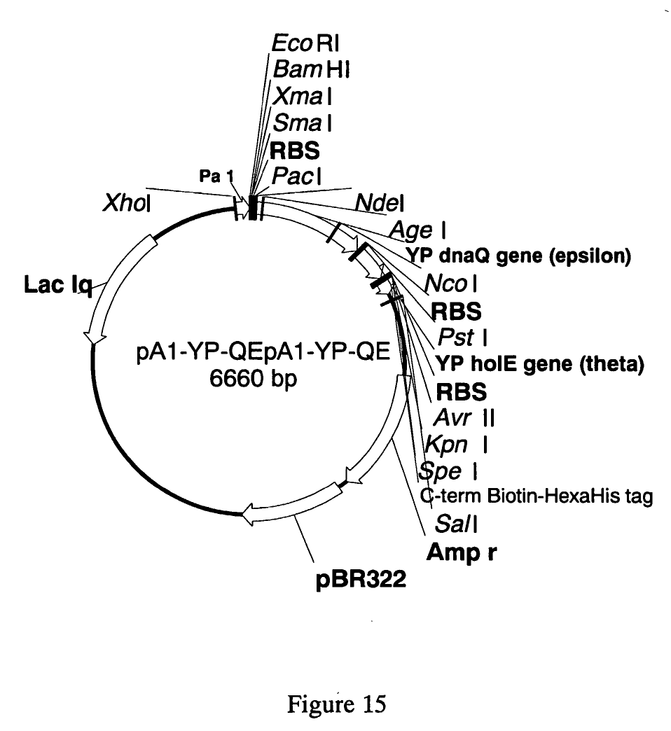

To determine if Y. pestis core could function in reconstitution assays, described above in Example 4.6, 1 μl of a 1:8 dilution of Fr II from the 45% ammonium sulfate precipitated sample or 1 μl E. coli core (3×10⁶ units/mg, 4 mg/ml) was assayed in reconstitution assays (25 μl) containing 1 μl E. coli β (1.7×10⁶ units/mg, 1 mg/ml), 4 μl τ complex (2.8×10⁶ units/mg, 2 mg/ml) and 19 μl of RNA primed M13Gori template. The results shown in FIG. 20 indicate that Y. pestis core can indeed function with E. coli β and E. coli τ complex.

Beta Clamp

### Example 15

### Molecular Cloning of the Y. pestis DnaN (β subunit) Processivity Factor

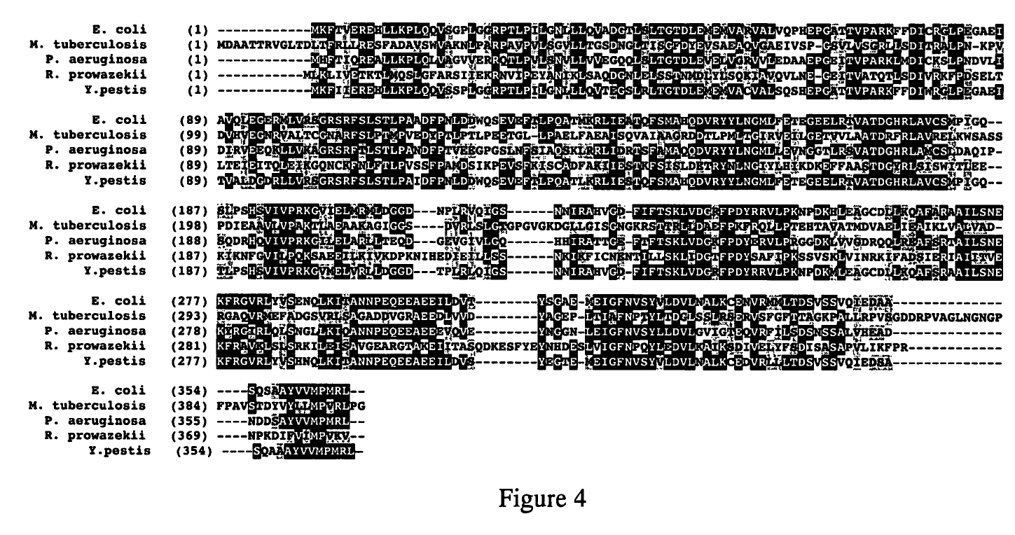

The gene for Y. pestis dnaN gene encoding the β subunit of DNA pol III holoenzyme was identified from the Y. pestis database (Sanger Database (2001) by its homology to other known DnaN (β) subunits. Comparison of amino acid sequences for DnaN proteins from Y. pestis and a selected subset of bacteria shows that they are highly homologous and align over the entire length of the protein sequence (FIG. 4).

This example describes PCR amplification of the dnaN gene (β subunit) out of the genomic DNA and inserting it into the vector pA1-CB-NdeI. The region of pA1-CB-NdeI encompassing the polyclonal region is shown in FIG. 10. The beta subunit is composed of 367 amino acids encoded by 1101 nucleotides of the dnaN gene. The nucleotide sequence of dnaN is represented by SEQ ID NO:18. The amino acid sequence of the β subunit is represented by SEQ ID NO:20. The forward/sense primer is: 5′-GATCcatATGAAATTTATCATTGAACGT-3′ (28-mer) (SEQ ID NO:21). The 5′ clamp (GATC) is followed by a NdeI restriction site shown in italic letters. The NdeI overlaps the ATG start codon and is used for insertion into pA1-CB-NdeI. The 21 nucleotides corresponding to the 5′ end of dnaN are shown in underlined capital letters.

The reverse/antisense primer is: 5′-GATCactagttcaCTACAAACGCATTGGCATG-3′ (32-mer) (SEQ ID NO:22). The 5′ clamp region (GATC) is followed by a SpeI restriction site for insertion into pA1-CB-NdeI (italic letters) and an additional stop codon (double underlined letters) in tandem with the native stop codon. Finally, the 19 nucleotides complementary to the 3′ end of the dnaN gene are shown in underlined capital letters.

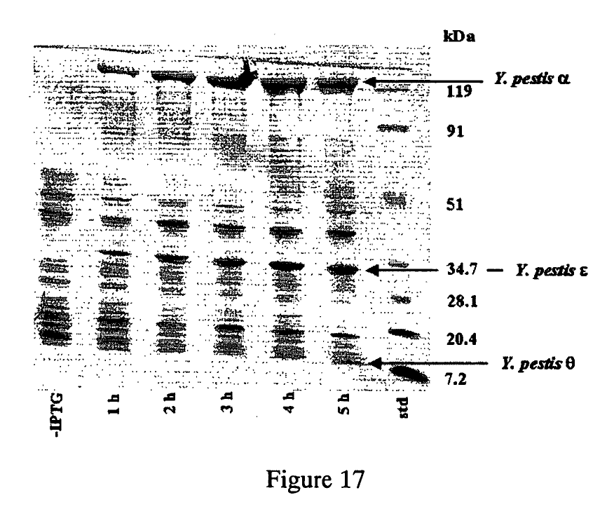

Both the dnaN PCR product and the plasmid pA1-CB-NdeI were digested with NdeI/SpeI restriction enzymes. The fragment of the PCR product containing the dnaN gene was inserted into the NdeI/SpeI pA1-CB-NdeI. This placed the dnaN gene optimally spaced from the upstream RBS and resulted in the plasmid pA1-YP-dnaN, which is schematically shown in FIG. 22. Sequencing of the dnaN insert confirmed the presence of native (unmutated) dnaN gene in the vector.

### Example 16

### Expression of dnaN in E. coli

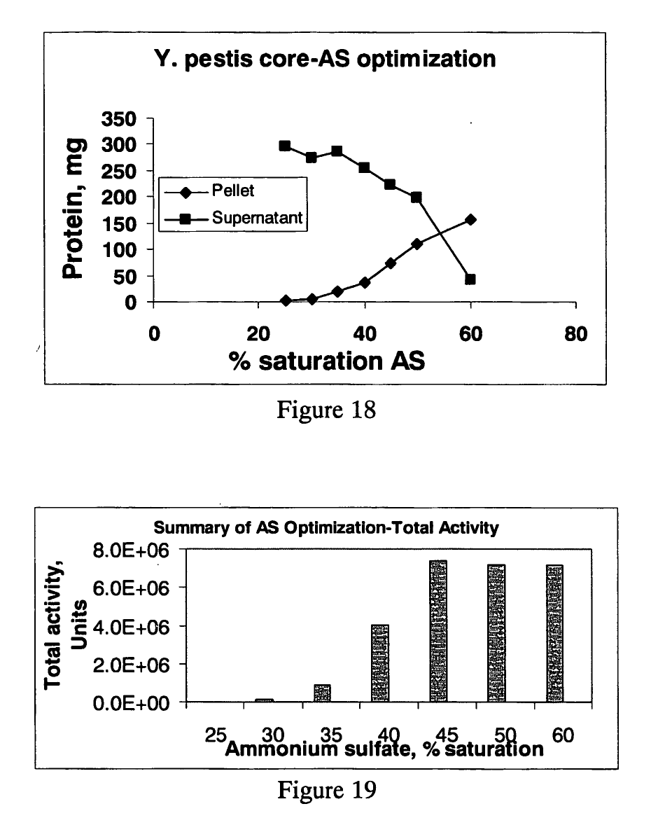

The correct sequence of the dnaN gene in pA1-YP-dnaN has been confirmed by DNA sequencing. The pA1-YP-dnaN plasmid was transformed into AP1.L1 strain of E. coli in the same manner as described in Example 4.1. SDS-polyacrylamide electrophoresis shows a protein band migrating at sites expected for β (40.9 kDa) in FIG. 23.

### Example 17

### Optimization of Expression of Y. pestis β in E. coli

In an attempt to optimize the yield of expressed recombinant Y. pestis β, induction times were analyzed for the pA1-YP-dnaN vector as described in Example 10. The results of the growth optimization analyzed by SDS-polyacrylamide gel electrophoresis for pA1-YP-dnaN is shown in FIG. 23. In this gel, E. coli β was used as a control as the molecular mass is almost identical with that of Y. pestis β. As can be seen in the gel, β is expressed to extremely high levels, approaching 50% of the total cellular protein! Molecular mass markers are shown on the right side of the gel.

### Example 18

### Large Scale Growth of β

Large-scale growth conditions were as described for the growth of core operon in Example 4.4, the length of growth time after induction for bacteria expressing β was 3 hours.

### Example 19

### Purification of β

Preparation of Fr I from 70 g of a 1:1 suspension of frozen cells (35 g cells) in Tris-sucrose was as described in Example 4.5 The recovered supernatant (110 ml) constituted Fraction I (Fr I) (30 mg/ml).

FrI was divided into seven samples of 15 ml each and labeled 25%, 30%, 35%, 40%, 45%, 50% and 60%. The protein in each sample was precipitated by adding varying amounts ammonium sulfate so that the final concentration of ammonium sulfate was: 25%, 30%, 35%, 40%, 45%, 50%, and 60% saturation, respectively, at 4 ° C. The mixture was stirred for an additional 30 min at 4° C. and the precipitate was collected by centrifugation (23,000×g, 45 min, 0° C.). The supernatant was removed from each sample and the resulting pellets were resuspended in buffer (25 mM Hepes (pH 7.5), 5 mM EDTA, 10% glycerol, 5 mM DTT, 100 mM NACl). The resulting mixture represents Fr II. The protein concentration of each sample from the resuspended pellets and the supernatants was determined using the Coomassie Protein Assay Reagent (Pierce) and bovine serum albumin (BSA) as a standard as shown in FIG. 24. The protein in the resuspended pellets was also analyzed using SDS-polyacrylamide gel analysis (not shown) and along with the protein assays indicated that Y. pestis β only precipitated in higher concentrations of ammonium sulfate (70%).

After ammonium sulfate precipitation conditions were optimized, lysis of 35 g of cells containing Y. pestis β to form Fraction I (FrI) (75 ml) was accomplished as described above. The protein in the sample was precipitated by ammonium sulfate so that the final concentration of ammonium sulfate was 70% saturation at 4° C. The mixture was stirred for an additional 30 min at 4° C. and the precipitate was collected by centrifugation (23,000×g, 45 min, 0° C.). The supernatant was removed from each sample and the resulting pellets were resuspended in 200 ml of TG.51/50 mm NaCl buffer (50 mM Tris-HCl (pH 7.0), 0.5 mM EDTA, 10% glycerol, 5 mM DTT plus 50 mM NaCl) and homogenized using a Dounce homogenizer. The sample was clarified by centrifugation (16,000×g) and the supernatant constituted Fr II (10.8 mg/ml). The protein concentration of each sample from the resuspended pellets was determined using the Coomassie Protein Assay Reagent (Pierce) and bovine serum albumin (BSA) as a standard. The sample was dilute to 1000 ml with TG.51 buffer containing no NaCl to adjust the conductivity to that of a DEAE chromatography column (200 ml, 5 cm×10 cm) equilibrated in TG.51/50 mM NaCl buffer. The sample was loaded onto the DEAE column, washed with 2.5 column volumes of TG.51/50 mM NaCl buffer and eluted with 10 column volumes of TG.51 buffer containing a 50-600 mM NaCl gradient. Fractions (40) were collected containing 25 ml each. The protein eluted in one peak between fractions 15-30 and 100% of the activity was recovered in fractions 20-26 (175 ml) containing Y. pestis β that was greater than 90% homogeneous. The protein in the sample was precipitated by ammonium sulfate so that the final concentration of ammonium sulfate was 70% saturation at 4° C. The mixture was stirred for an additional 30 min at 4° C. and the precipitate was collected by centrifugation (23,000×g, 45 min, 0° C.), quick frozen in liquid nitrogen and stored at −80° C.

The protein pellets containing Y. pestis β were resuspended in imidazole buffer (50 mM imidazole, (pH 6.8), 10% glycerol, 50 mM NaCl, 5.0 mM DTT) and homogenized using a Dounce homogenizer. The sample was clarified by centrifugation (16,000×g) and the resulting sample constituted FrIII. The sample was diluted (460 ml) using Imidazole buffer minus NaCl to the conductivity of a hydroxylapatite chromatography column (200 ml, 5cm×10 cm) equilibrated in imidazole buffer. The sample was loaded onto the hydroxylapatite column, washed with 3 column volumes of Imidazole buffer/plus 10 mM KPO₄ and eluted with 10 column volumes of imidazole buffer containing a 20-130 KPO₄ gradient. Fractions (80) were collected containing 25 ml each. The protein and the activity eluted in a single peak. Fractions 25-35 were pooled (275 ml) and contained 95% of the total activity loaded onto the column. The protein in the sample was precipitated by ammonium sulfate so that the final concentration of ammonium sulfate was 70% saturation at 4° C. The mixture was stirred for an additional 30 min at 4° C. and the precipitate was collected by centrifugation (23,000×g, 45 min, 0° C.), quick frozen in liquid nitrogen and stored at −80° C.

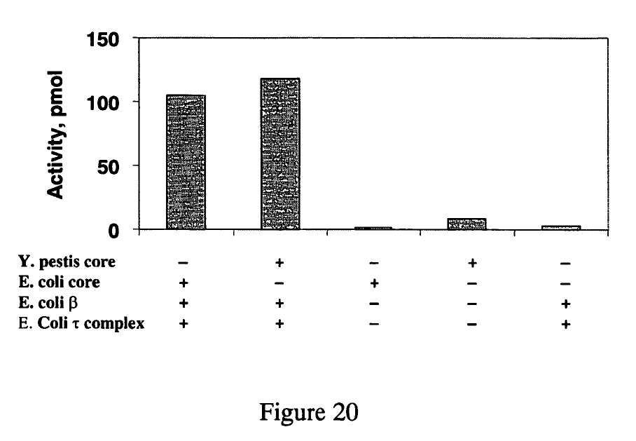

The protein pellets containing Y. pestis β was resuspended in HG.05 buffer (25 mM HEPES, (pH 7.5), 50 mM KCl, 10% glycerol, 5 mM DTT and 1 mM EDTA) and homogenized using a Dounce homogenizer. The sample was clarified by centrifugation (16,000×g) and the resulting sample was Fr IV. The sample was loaded onto a Sephacryl® S-200 (Amersham Pharmacia Biotech) chromatography column (250 ml, 1:20 ratio) equilibrated with HG.05 buffer. The procedure included running the buffer above the resin down to the resin bed, gently pipetted the sample onto the resin, pumping the sample into the resin, then 2 ml of buffer was gently added onto the top of the resin and pumped into the resin. The running buffer was then added to the top of the resin and elution was continued. The protein was eluted at a flow rate of 0.22 ml/min and 2 ml fractions were collected. Fractions 45-55 (22 ml) contained all of the loaded activity and were pooled (Fr V). Fr V was aliquoted and stored at −80° C. and represents the stock supply of Y. pestis β. At each step of the purification process the purity of Y. pestis β was analyzed by SDS polyacrylamide gel electrophoresis. A summary gel of the pooled protein for each purification step is shown in FIG. 25.

The protein concentration and activity was also characterized at each purification step (Table III).

TABLE III

| | | | Protein | | Specific |
|---|---|---|---|---|---|
| | Volume | | concentration | Activity | Activity |
| Fraction | (ml) | Protein (mg) | (mg/ml) | (Units) | (Units/mg) |
| Fr I | 75 | 2610 | 34.8 | — | — |
| Fr II | 200 | 2164 | 10.8 | 1.6 × 10¹⁰ | 7.4 × 10⁶ |
| Fr III | 460 | 632 | 1.4 | 1.0 × 10¹⁰ | 1.6 × 10⁷ |
| Fr IV | 7 | 411 | 58.7 | 9.5 × 10⁹ | 2.3 × 10⁷ |
| Fr V | 22 | 440 | 20.0 | 1.3 × 10¹⁰ | 3.0 × 10⁷ |

### Example 20

### Characterization of Processivity Activity via Reconstitution with E. coli Core and Tau Complex

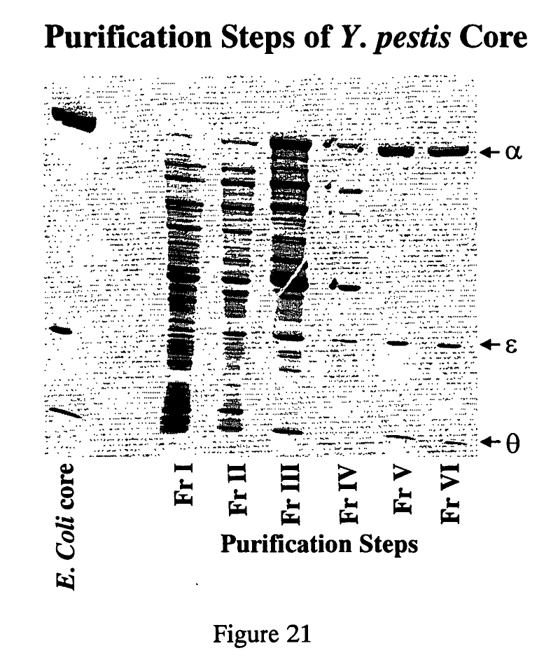

To determine if Y. pestis β could stimulate E. coli core and τ complex, assays were carried out in which Y. pestis β from the 35% AS precipitated protein was titrated into E. coli reconstitution assays as described in Example 4.6. The reconstitution assays (26 μl) contained 1 μl E. coli core (3×10⁶ units/mg, 4 mg/ml), 4 μl τ complex (2.8×10⁶ units/mg, 2 mg/ml) and 19 μl of RNA primed M13Gori template. To this is added 2 Al of various dilutions of Fr II (35% AS precipitation) containing Y. pestis μ. The results of these assays are shown in FIG. 26.

These results show that Y. pestis μ subunit in FrII is active since it can fully stimulate rapid and processive DNA syntesis in the presence of pol III and τ complex from E. coli. These same dilutions of Fr II (35% AS precipitation) was assayed in the absence of E. coli pol III and found to contain only very low levels of background activity.

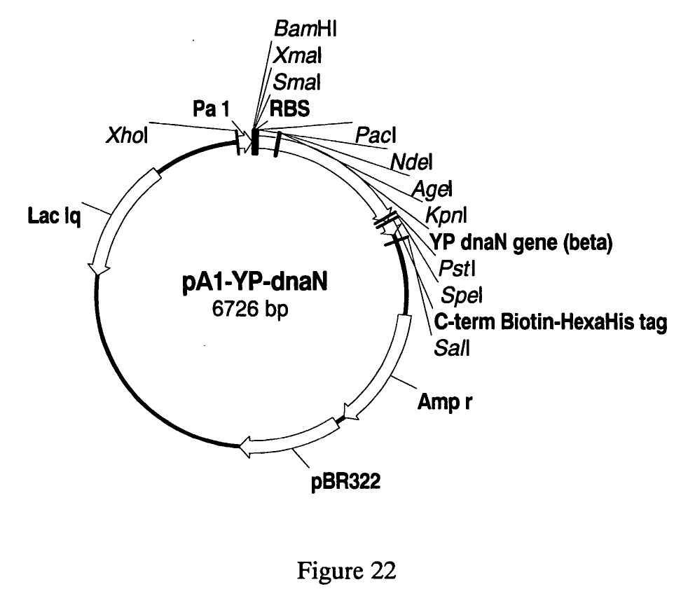

These same assays were initially used to confirm the ammonium sulfate precipitation optimizations described in Example 19. These assays were carried out for all seven Fr II samples resulting from the different concentrations of ammonium sulfate precipitation of protein described in Example 19. The results confirm that Y. pestis β is most efficiently precipitated out of solution at high concentrations of ammonium sulfate even though many endogenous contaminating proteins are also precipitated (FIG. 27).

Clamp Loading Complex

### Example 21

### Molecular Cloning of Y. pestis dnaX, the Gene Encoding the Gamma and Tau Subunits

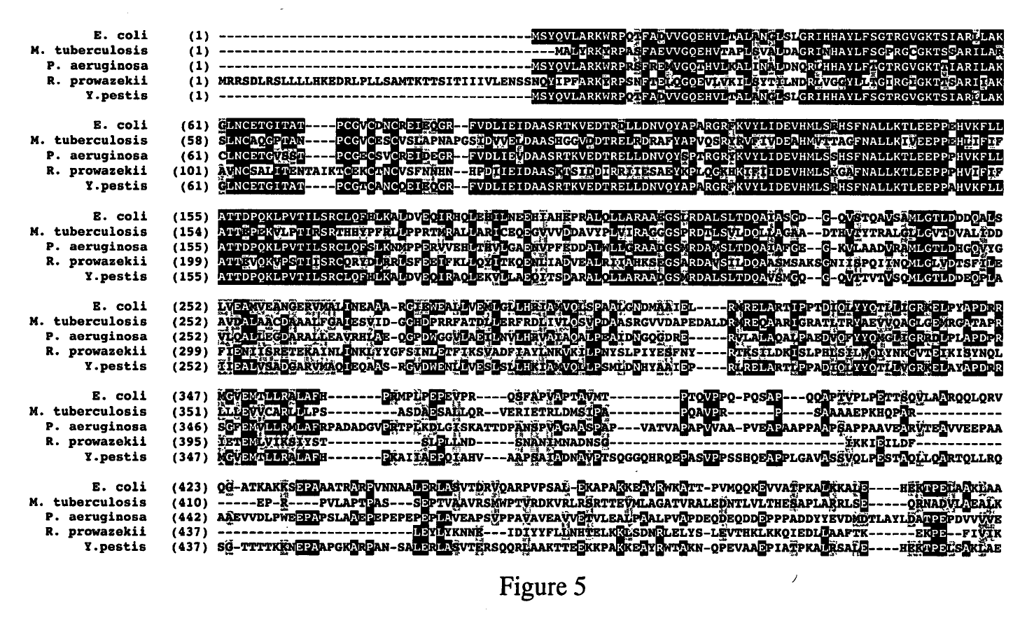

The gene for Y. pestis dnaX gene of DNA pol III holoenzyme was identified by its homology to other known DnaX subunits from the Y. pestis database (Sanger database (2001)). Comparison of amino acid sequences for DnaX proteins from Y. pestis and a selected subset of bacteria shows that they are homologous and align over the functional region of the protein sequence, with the highest sequence identity occurring in the amino-terminal region of the protein (FIG. 5).

This example describes PCR amplification of the dnaX gene (τ) out of the genomic DNA and inserting it into the vector pA1-CB-NcoI. The τ subunit is composed of 659 amino acids SEQ ID NO:25 encoding by 1977 nucleotides. The nucleotide sequence of dnaX is represented by SEQ ID NO:23. Expression of the dnaX gene in E. coli results in two natural products; τ, the full-length product and γ, a truncated product. γ is the C-terminally truncated variant of τ formed as a result of −1translational frameshifting. The −1 frameshift occurs at a sequence of six A's. The same frameshift sequence is present in the nucleotide sequence of Y. pestis dnaX gene. To force the expression of only the full-length protein product (τ), three nucleotides in the dnaX gene were mutated. The mutations are 1329A>G, 1332G>A and 1335T>C. The 1329A>G eliminates the −1 frameshift, the 1332G>A replace a low usage codon with a high usage codon, and the 1335T>C mutation eliminates the stop codon in case of a −1 frameshift. These mutations maintain the correct sequence of the protein while eliminating the frameshift, as shown below.

| | | |
|---|---|---|
| tau expression | | gamma expression |
| Lys Lys Asn Glu | | Lys Lys Glu Stop |
| (SEQ ID NO:26) | | |
| A AAA AAG AAT | the −1 frame- | AAA AAA GAA |
| GAG ... | shift causes | TGA ... |
| (SEQ ID NO:27) | | (SEQ ID NO:28) |
| the mutation will | tau expression | |
| only allow | | |
| Lys Lys Asn Glu | (SEQ ID NO:29) | |
| A AAG AAA AAC | (SEQ ID NO:30) | |
| GAG ... | | |

The amino acid sequence of τ is represented by SEQ ID NO:25. The cloning of dnaX results from two separate steps, during which the above described mutations are introduced.

In the first step there are two PCR reactions, PCR#1 and PCR#2. In the PCR#1 reaction, the forward/sense primer at the 5′ end of the dnaX gene was: 5′-GATCttaattaagagctccccATGAGCTATCAGGTCCTTG-3′ (40-mer) (SEQ ID NO:31). The GATC clamp is followed by a PacI restriction site (italic letters) and a SacI restriction site (underlined italic letters). The PacI site is needed for insertion into pA1-CB-NcoI and the SacI site is used subsequently for building the 5-gene operon (see below). The 3-base spacer (boldface ccc) is needed for optimal spacing between the upstream RBS and the dnaX start codon in the 5-gene operon. The 3′ 19 nucleotides are complementary to the 5′ end of the dnaX gene. The reverse/antisense primer containing mutations was 5′-GCTCGTTTTCTTTGTFGTGGTCGTCCCCG-3′ (SEQ ID NO:136). This PCR fragment contained the 5′ 1359 nucleotides from dnaX.

In the PCR#2 reaction, the forward/sense primer contained the mutations. The forward/sense primer was 5′-CAAAGAAAAACGAGCCGGCGGCGCCAGGAAAA-3′. The reverse/antisense primer is: 5′-GATCactagtttaTTAAATTGGTCGGATAC-3′ (30-mer) (SEQ ID NO:32). The reverse/antisense primer contained a GATC clamp is followed by a SpeI restriction site and an additional stop codon (double underlined letters). This is followed by 17 nucleotides complementary to the 3′ end of the dnaX gene.

In step 2, the two PCR fragments from step 1 were mixed and served as a template and the forward/sense primer from PCR#1 reaction and the reverse/antisense primer from PCR#2 reaction were used in PCR#2 reaction. This reaction resulted in a PCR product containing the full-length dnaX gene, mutated at the appropriate sites.

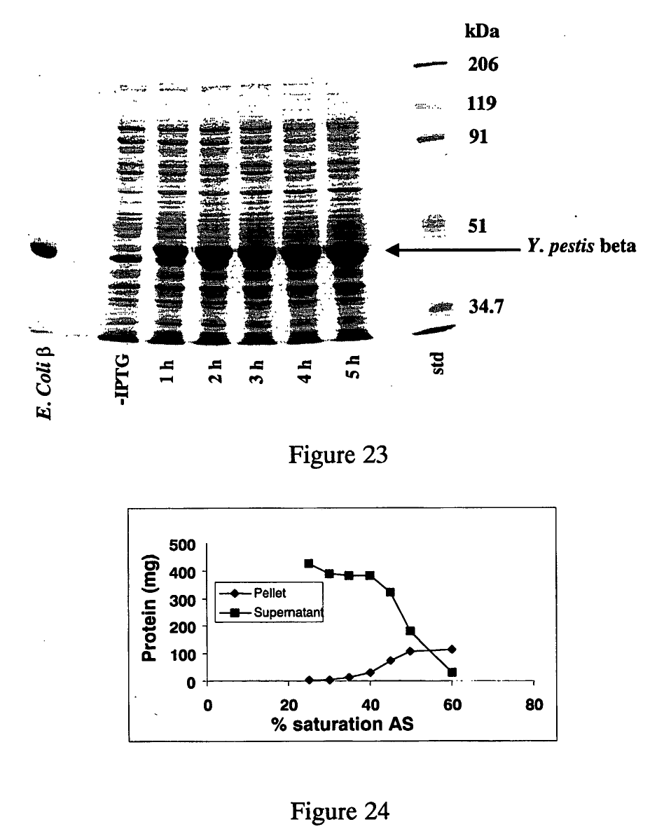

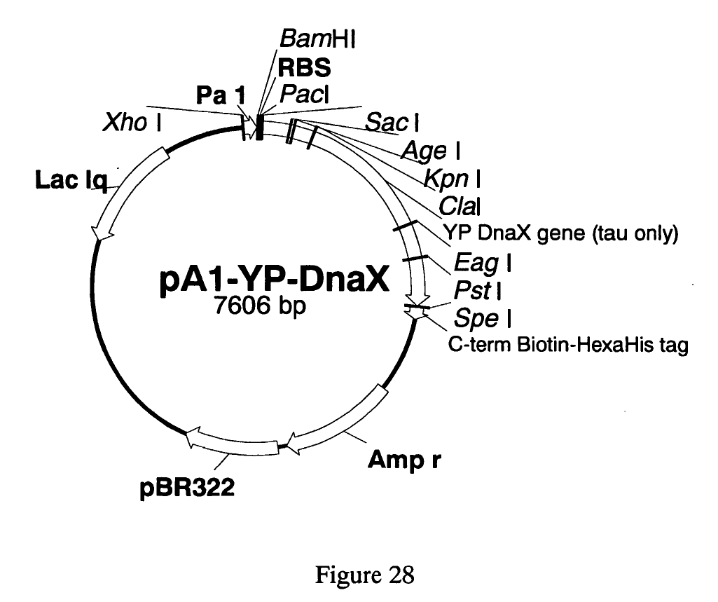

Both the PCR#3 product and the plasmid pA1-CB-NcoI are digested with PacI/SpeI restriction enzymes. The fragment of the digested PCR product containing the dnaX gene is inserted into the digested pA1-CB-NcoI plasmid (the cloning region of pA1-CB-NcoI is shown in FIG. 31) to produce plasmid pA1-YP-dnaX (shown in FIG. 28), which contains the dnaX gene containing the three mutated nucleotides. Sequencing of the dnaX insert has been completed confirming the presence of correctly mutated dnaX gene in the vector. The SacI and SpeI restriction sites are used subsequently in the formation of the 5-gene operon.

### Example 22

### Purification and Characterization of Tau Subunit

The Y. pestis DnaX protein will be purified using a reconstitution assay to monitor the purification. Although complex, these procedures have become more commonplace and are used in E. coli, T. thermophilus (as described in copending U.S. patent application Ser. No. 09/818,780, filed Mar. 28, 2001) and S. pyogenes (as described in International Application Serial No. PCT/US01/48396, filed Oct. 29, 2001) DNA replication studies. Routinely, expression at adequate levels can be obtained such that the protein can be observed using SDS-PAGE. If this is not realized in these studies, expression may be verified with monoclonal antibodies directed against E. coli subunits. Significant cross-reactivity has been observed with selected antibodies against the holoenzyme subunits of more evolutionarily distant organisms (McHenry, C. S., Seville, M., and Cull, M. G. (1997) J Mol Biol 272, 178-189) in the past. Once overproducers of the DnaX subunit is in hand, cells will be lysed and ammonium sulfate pellets will be generated using optimal concentration of saturated ammonium sulfate initially to guarantee precipitation of the sought subunits. The redissolved, dialyzed, pellets will be used for reconstituting Y. pestis DNA polymerase III holoenzyme to provide the basis for a quantitative functional assay to direct the purification of individual subunits.

### Example 23

### Molecular Cloning of Y. pestis holA, the Gene Encoding the Delta Subunit Protein

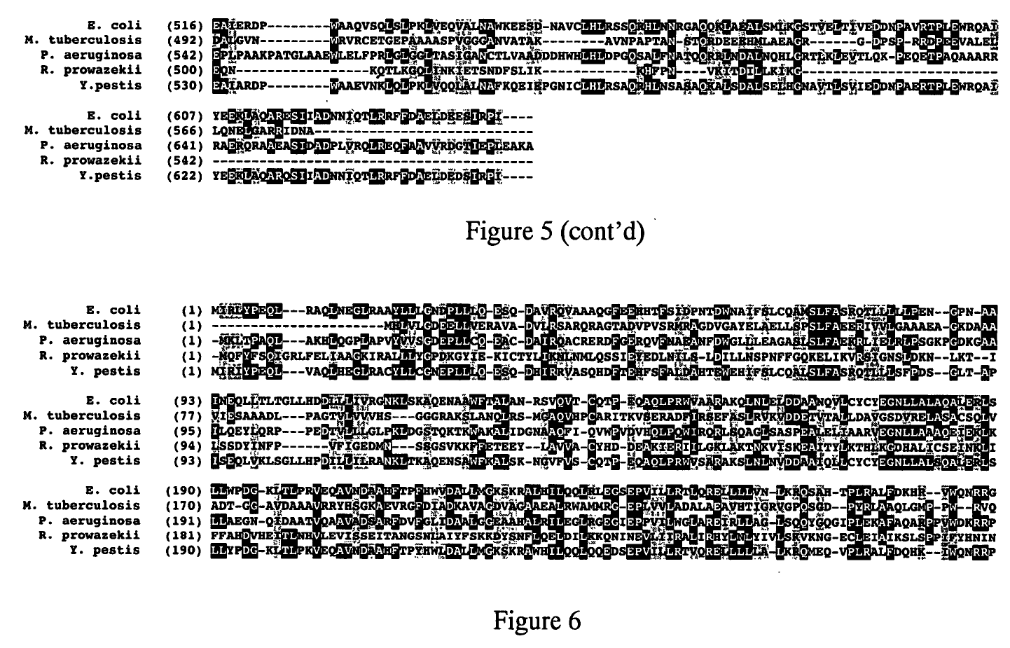

The Y. pestis holA gene of DNA pol III holoenzyme was identified from the Y. pestis database (100) by its homology to other known HolA (6) subunits. Comparison of amino acid sequences for 8 subunits from Y. pestis and a selected subset of bacteria show that they have low homologous over the entire length of the protein sequence (FIG. 6).

This example describes PCR amplification of the holA gene (6 subunit) out of genomic DNA and inserting it into the vector pA1-CB-NcoI. The 6 subunit is composed of 345 amino acids encoded by 1035 nucleotides. The nucleotide sequence of holA is represented by SEQ ID NO:33. Codon 3 is a low usage codon in E. coli (CGG) and will be changed to a high usage codon (CGT). The amino acid sequence of delta is represented by SEQ ID NO:35. The forward/sense primer is:

| | | |
|---|---|---|
| 5′-GATCttaattaagcttcccATGAT | (47-mer) | (SEQ ID NO:36) |
| CCGTATTTACCCTGAACAACTTG-3′. | | |

The GATC clamp is followed by a PacI restriction site (italic letters) that overlaps a HindIII restriction site (underlined italic letters). Next, there is a 3-base spacer (ccc, boldface letters) for optimal spacing between the RBS and holA gene during operon formation. The spacing in this construct between the RBS and holA start codon will be 15 nucleotides. Following the 3-base spacer are the first three codons of holA (capitalized italic letters) that will not be complementary because of the modification of codon 3 to a high usage codon as described above. The last 19 nucleotides (underlined capital letters) correspond to the 5′ end of holA gene beginning at codon 4. The reverse/antisense primer is: 5′-GATCactagtcccgagctccctcctttaTTAATGGGCATCAAAAAAGCTC-3′ (50-mer) (SEQ ID NO:37). The GATC clamp is followed by a SpeI restriction site (italic letters) separated from a SacI restriction site (underlined italic letters) by a 3-base spacer (boldface ccc) for efficient digestion with both SpeI and SacI. RBS sequence follows. The SacI and SpeI sites and the RBS are needed for construction of the 5-gene operon. Next, is a second stop codon (double underlined letters) in tandem with the native stop codon. Finally the last 22 nucleotides (underlined capital letters) are complementary to the holA 3′ end including the native stop codon.

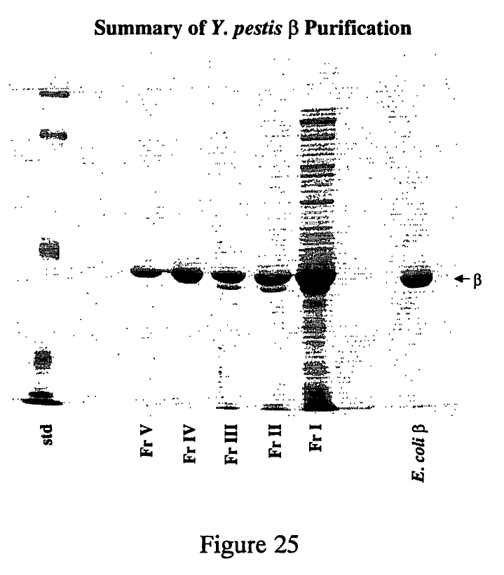

Both the PCR product and the plasmid pA1-CB-NcoI were digested with PacI/SpeI restriction enzymes. The fragment of the digested PCR product containing the holA gene was inserted into the digested pA1-CB-NcoI plasmid (cloning region of pA1-CB-NcoI is shown in FIG. 31). This results in the formation of the plasmid pA1-YP-holA, which contains the holA gene followed by an RBS for the gene placed downstream in formation of the 5-gene operon. Sequencing of the holA insert has been completed confirming the presence of native (unmutated) holA gene in the vector. The HindIII and Sac I/SpeI will be used in the formation of the 5-gene operon. This plasmid, referred to as pA1-YP-holA, and is shown schematically in FIG. 29.

### Example 24

### Purification and Characterization of HolA (Delta) Subunit

Purified DnaE, DnaN, DnaX and HolB will be used to provide an assay for HolA. HolA will be purified using the logic described for the other proteins, until highly purified.

### Example 25

### Molecular Cloning of Y. pestis holB, the Gene Encoding the HolB (Delta Prime) Subunit

The gene for Y. pestis holB gene of DNA pol III holoenzyme were identified from the Y. pestis database (100) by its homology to other known HolB subunits. Comparison of amino acid sequences for 8′ subunits from Y. pestis and a selected subset of bacteria shows that they are homologous and align over the entire length of the protein sequence (FIG. 6).

This example describes PCR amplification of the holB gene (6′) out of genomic DNA and inserting it into the vector pA1-CB-NcoI. The 8′ subunit is composed of 341 amino acids encoded by 1023 nucleotides. The nucleotide sequence of holB is represented by SEQ ID NO:38. Codon 2 encodes Asn. This is a low usage codon in E. coli and is therefore modified to a high usage codon in the PCR reaction. The codon is AAT and in the PCR reaction is changed to AAC. The amino acid sequence of 6′ is represented by SEQ ID NO:40. To carry out the PCR reaction the forward/sense primer is: 5′-GATCttaattaacctaggcccATGAACTGGTATCCGTGGCTTAACGC-3′ (47-mer) (SEQ ID NO:41). The GATC clamp is followed by restriction sites for PacI (italic letters) and AvrII (underlined italic letters). A 3-base spacer that follows (boldface “ccc”) allows for optimal spacing between an upstream RBS and the start codon of holB in the 5-gene operon. The spacing between the RBS and holB start codon for individual expression here is 17 nucleotides, which is somewhat longer than optimal, but it is expected to be functional. The first two codons that are not complementary and contain the modified codon 2 described above are shown in capital italic letters. The remaining 20 nucleotides are complementary to the 5′ end of holB beginning at codon 3 and are shown in underlined capital letters. The reverse/antisense primer is: 5′-GATCggtaccaagcttcctcctttaTTACAACGAAGGTAAGGTG-3′ (44-mer) (SEQ ID NO:42). The GATC clamp is followed by restriction sites for KpnI (italics) and HindIII (underlined italic letters). A RBS sequence (boldface letters) and a second stop codon (double underlined letters) follow. The additional stop codon is followed by 20 nucleotides containing the native stop codon that is complementary to the 3′ end of the holB gene. The KpnI site is used to insert holD into pA1-CB-NcoI and the HindIII site will be used in the formation of the 5-gene operon.

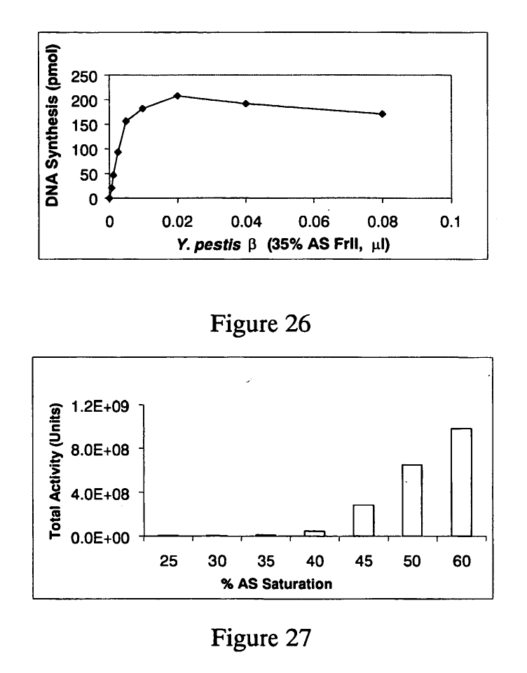

Both the PCR product and the plasmid pA1-CB-NcoI were digested with PacI/KpnI restriction enzymes. The fragment of the digested PCR product containing the holB gene was inserted into the digested pA1-CB-NcoI plasmid. This resulted in the formation of the plasmid pA1-YP-holB (FIG. 30), which contains the holB gene followed by an RBS for the gene placed downstream in the 5-gene operon (shown below). Sequencing of the holB insert has been completed confirming the presence of native (unmutated) holB gene in the vector. The AvrII and Hin dIII restriction sites will be used in the formation of the 5-gene operon. This plasmid, referred to as pA1-YP-holB, and is shown schematically in FIG. 30.

### Example 26

### Purification and Characterization of Y. pestis HolB (Delta Prime Subunit)

Hol B will be purified using purifications for E. coli HolB (δ′) and T. thermophilus HolB as a model.

### Example 27

### Molecular Cloning of Y. pestis holC, the gene encoding HolC (Chi) Subunit Protein

This example entails PCR amplification of the holC gene (χ subunit) out of the genomic DNA and inserting it into the pA1-CB-NcoI vector. The χ subunit is relatively small (149 amino acids, encoded by 450 nucleotides including a stop codon). All of the primers used in PCR amplification have been purified by either polyacrylamide gel electrophoresis or by HPLC. The nucleotide sequence of holC gene is represented by SEQ ID NO:43. The start and stop codons are shown in boldface letters here and throughout this document. The amino acid sequence of the χ subunit is represented by SEQ ID NO:45. To PCR the holC gene, the forward/sense primer used is 5′-GATCttaattaacATGAAAAACGCTACCTTCTAC-3′ (34-mer) (SEQ ID NO:46). There is a GATC clamp region on the 5′ end of the primer to allow for efficient digestion with restriction enzymes. Next, there is a PacI restriction site, shown in italic letters. There is a “c” (boldface and double underlined) between the PacI site and the ATG start codon for proper spacing between the ribosome binding site (RBS), AGGAGG, located upstream of the PacI site and the start codon. The region complementary to the 5′ end of the holC gene is composed of 21 nucleotides and is shown in capitalized and underlined letters. The reverse/antisense primer used is: 5′-GATCggtacccctccttcaTCAATTAGTTGGCGGCG-3′ (36-mer) (SEQ ID NO:47). There is a GATC clamp on the 5′ end for efficient digestion by restriction enzymes. Next is a KpnI site, shown in italic letters. Next, a RBS site that is used an operon for the downstream gene is made. A second non-complementary stop codon is next, shown in double underline. The complementary region of the primer begins with the natural stop codons and is complementary to 17 nucleotides on genomic DNA. This is shown as capitalized and underlined letters. The region of the pA1-CB-NcoI vector that contains the polyclonal site is shown in FIG. 31.

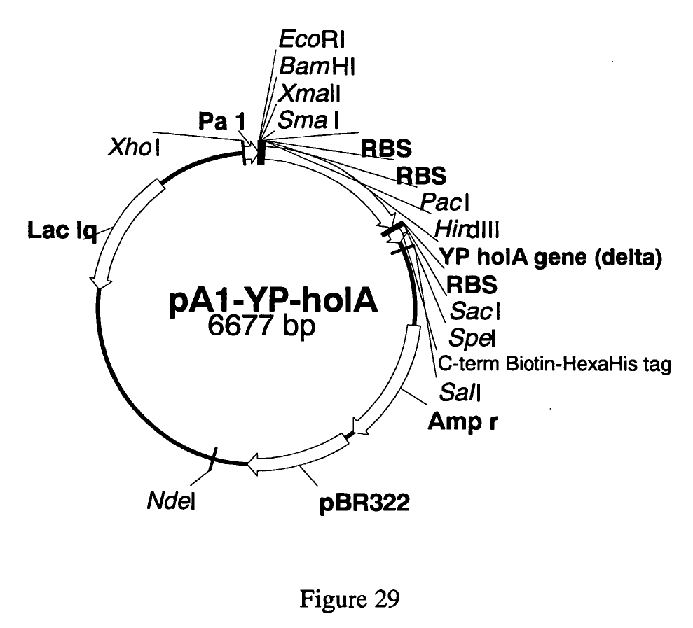

Both the PCR product and the plasmid pA1-CB-NcoI were digested with PacI/KpnI restriction enzymes. The fragment of the PCR product containing the holC gene was inserted into the digested pA1-CB-NcoI plasmid. This created pA1-YP-holC, which contains a RBS sequence just downstream of the holC gene, which can be utilized by the next downstream gene as an operon is built. Schematic representation of pA1-YP-holC vector is shown in FIG. 32.

### Example 28

### Molecular Cloning of Y. pestis holD, the Gene Encoding HolD (psi) Subunit Protein

This example describes PCR amplification of the holD gene (ψ subunit) out of the genomic DNA and inserting it into the pA1-CB-NcoI vector. The ψ subunit is also relatively small (146 amino acids, encoded by 438 nucleotides). The nucleotide sequence of holD gene is represented by SEQ ID NO:48. Codons 3, 4 and 5 (shown in the nucleotide sequence in underlined letters) which code for Ser Arg Arg are low usage codons in E. coli and will be changed to high usage codons in the PCR reaction. The change will be from TCAAGACGA to TCCCGCCGT. The amino acid sequence of ψ is represented by SEQ ID NO:50. To PCR the holD gene, the forward/sense primer was: 5′-GATCttaattaaggtacctccATGGCATCCCGCCGTGACTTGCTGTTACAGCAG-3′ (54-mer) (SEQ ID NO:51). The GATC clamp is followed by a PacI site shown in italic letters. Adjacent to the PacI site is a KpnI site, shown in underlined italic letters followed by a “tcc” spacer shown in boldface letters. This spacer lengthens the region between the RBS in pA1-CB-NcoI and the start codon of holD to 17 nucleotides, which is well above the optimal spacing, but ψ subunit is not expected to be soluble in the absence of χ subunit, based on the E. coli precedent, and will not be expressed alone. The spacer does however optimally position the start of holD from the RBS that is downstream of holC in pA1-YP-holC when the holCD operon is made. The following 15 nucleotides encompassing codons 1-5, which do not exactly correspond to the 5′ end of holD gene because of the changes described above, are shown as capital italic letters. Finally, the 18 nucleotides that correspond to codons 6-12 are shown in underlined capital letters.

The reverse/antisense primer is: 5′-GATCactagtccccctaggcctccttcaTCAATTTGGTATGCTGTG-3′ (46-mer) (SEQ ID NO:52).There is a GATC clamp for efficient restriction digestion at the 5′ end of the primer. Next are the SpeI and AvrII restriction sites (shown as underlined italic letters) separated by a three base spacer (bold-“ccc”). This three base spacer is needed to allow for digestion by both SpeI and AvrII later during operon construction. The restriction sites are followed by a RBS sequence in bold and an additional (second) stop codon (double underlined letters), which will be adjacent to the native stop codon. Finally, the 18 nucleotides that are complementary to the 3′ end of the hold gene, including the native stop codon are shown in underlined capital letters. The SpeI site was used to insert hold into pA1-CB-NcoI and the AvrII site will be used later in the formation of the 5-gene operon.

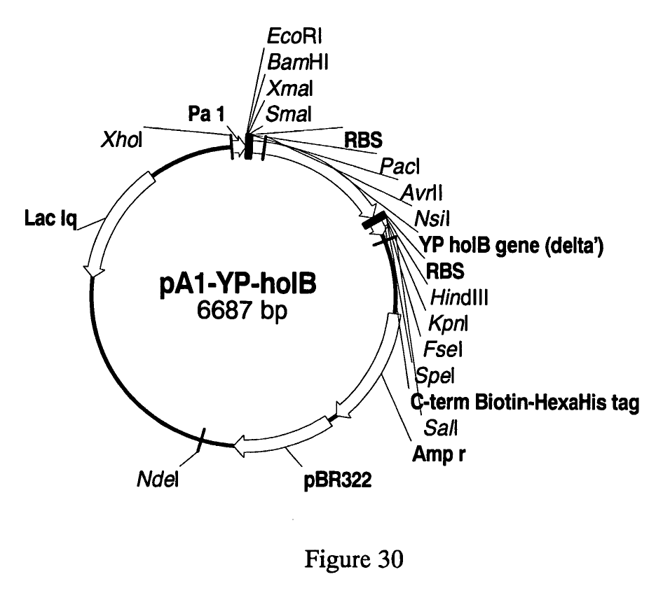

Both the PCR product and the plasmid pA1-CB-NcoI were digested with PacI/SpeI restriction enzymes. The fragment of the digested PCR product containing the holD gene was inserted into the digested pA1-CB-NcoI plasmid, resulting in the formation of the plasmid pA1-YP-holD, which contains the holD gene followed by an RBS for the gene placed downstream in formation of the 5-gene operon. Sequencing of the holD insert has been completed confirming the presence of native (unmutated) holD gene in the vector. Schematic representation of pA1-YP-holD vector is shown in FIG. 33.

Operon Cloning

### Example 29

### Construction of 2-Gene Operon Containing Both holC and holD

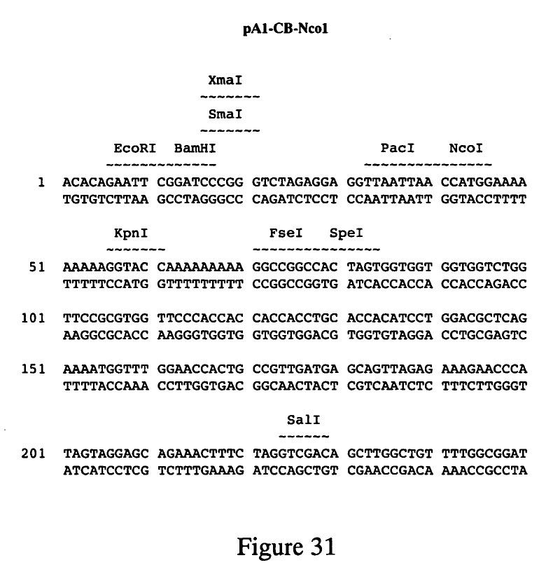

This example describes the formation of a 2-gene operon that contains both holC and holD genes in the same expression vector. This is accomplished by digesting both pA1-YP-holC and pA1-YP-holD with KpnI and SpeI. The small fragment from pA1-YP-holD that contains the holD gene between the KpnI and SpeI sites was inserted into the digested pA1-YP-holC plasmid. This placed hold downstream of holC with optimal spacing from the RBS created in the holC construct. This plasmid, referred to as pA1-YP-holCD, and is shown schematically in FIG. 34.

### Example 30

### Construction of 2-Gene Operon Containing Both holB and holA

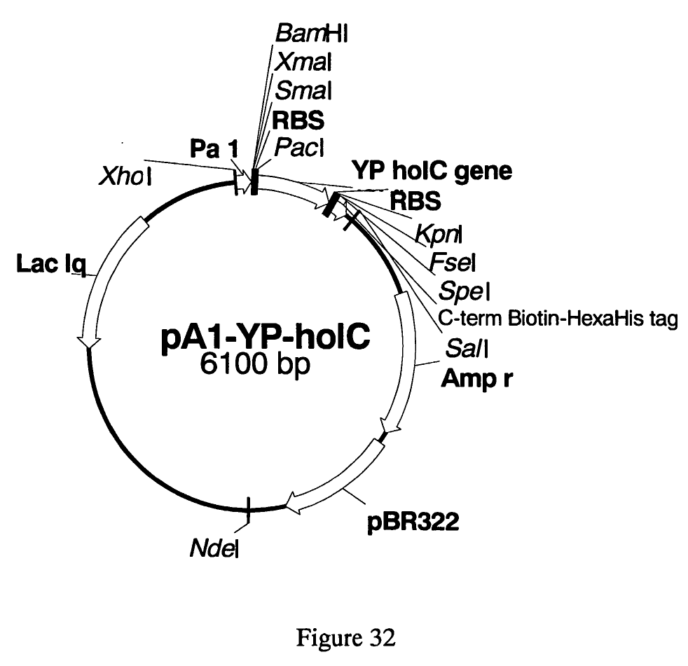

This example describes the formation of a 2-gene operon containing holB and holA genes. This is accomplished by digesting both pA1-YP-holB and pA1-YP-holA with Hin dIII and SpeI. The small fragment from pA1-YP-holA that contains the holA gene between the HindIII and SpeI sites is inserted into the digested pA1-YP-holB plasmid. This places holA downstream of holB and also downstream with optimal spacing from the RBS created in pA1-YP-holC (χ vector). This plasmid is referred to as pA1-YP-holBA, and is shown schematically in FIG. 35.

### Example 31

### Construction of 3-Gene Operon Containing holB, holA, and dnaX

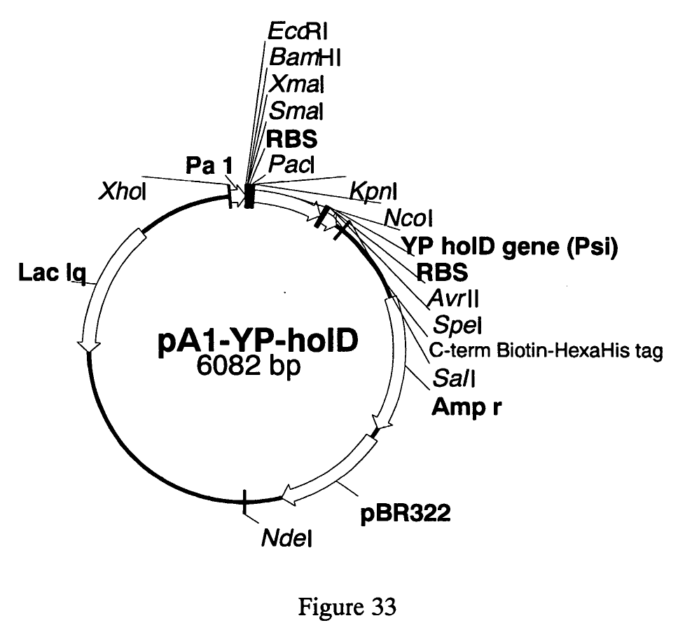

This example describes the preparation of a 3-gene operon containing holB, holA and dnaX. This is accomplished by digesting both pA1-YP-holBA and pA1-YP-dnaX with SacI and SpeI. The small fragment from pA1-YP-dnaX that contains the dnaX gene between the SacI and SpeI sites was inserted into the digested pA1-YP-holBA plasmid. This places the dnaX gene downstream of the holA gene and also downstream with optimal spacing from the RBS created in the holA vector construct. This plasmid is referred to as pA1-YP-holBAX and is schematically shown in FIG. 36.

### Example 32

### Construction of 5-Gene Operon holC, holD, holB, holA, and dnaX

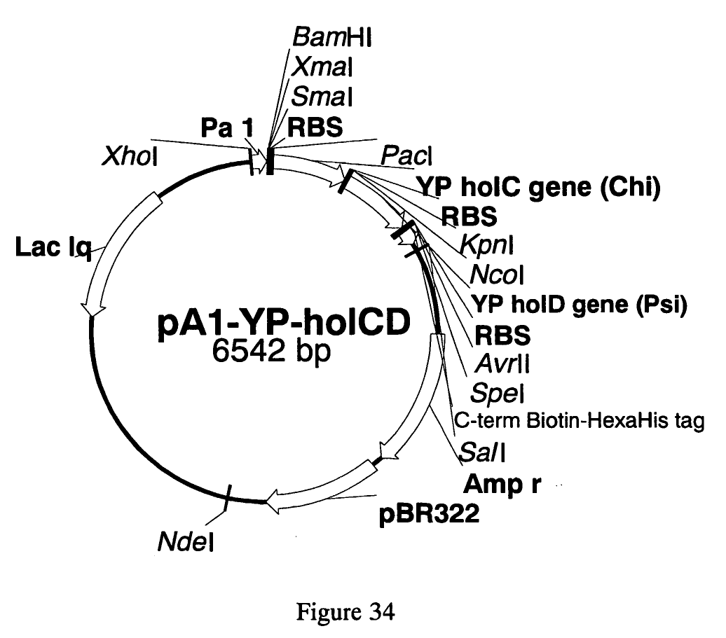

This example describes the combination of the 2-gene operon containing holC and holD genes (χ and ψ, pA1-YP-holCD vector) with the 3-gene operon containing holB, holA and dnaX (δ′, δ and τ, pA1-CB-holBAX vector) to produce a 5-gene operon composed of all the subunits needed to form the clamp-loading complex. This plasmid is referred to as pA1-YP-CLcomplex. Both pA1-YP-holCD and pA1-YP-holBAX were digested with AvrII/SpeI. The AvrII/SpeI fragment from the digested pA1-YP-holBAX containing the 3-gene operon was inserted into the AvrII/SpeI-digested pA1-YP-holCD. This placed the 3-gene operon downstream of the 2-gene operon. The RBS placed downstream of the holD gene serves as the upstream RBS for the holB gene. This produces a 5-gene operon with each gene optimally spaced from its own unique RBS (pA1-YP-CLcomplex). This is schematically shown in FIG. 37.

### Example 33

### Expression of Y. pestis Clamp Loader Complex

With the construction of pA1-YP-CLcomplex completed, the correct sequences of all genes have been confirmed by DNA sequencing (not shown).

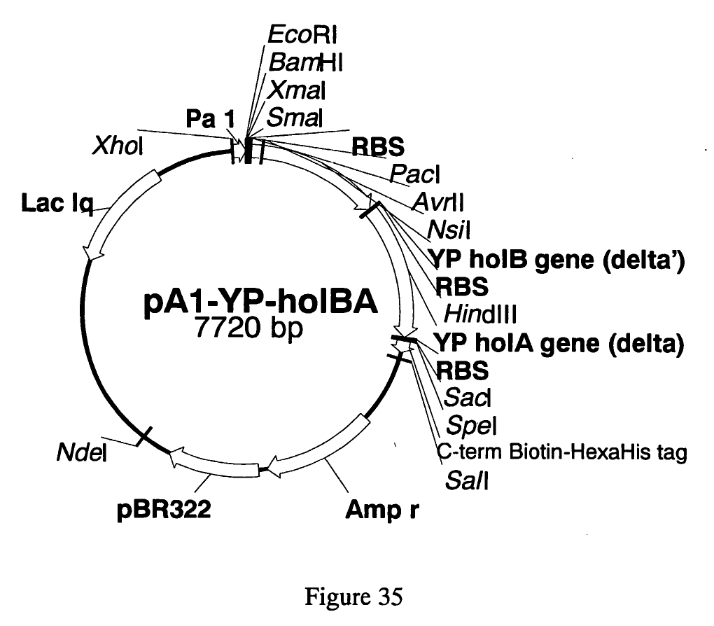

The plasmid was transformed into AP1.L1 strain of E. coli and expressed as described for the core operon in EXAMPLE 9.3. A Coomassie Blue stained protein gel in FIG. 38 shows protein bands migrating at sites expected for proteins with molecular masses corresponding to δ (39.6 kDa), δ′ (38.2 kDa), χ (17 kDa), ψ (16.2 kDa) and τ (72.3 kDa) subunits, which could be detected as distinct protein bands, but were not observed in the uninduced control. The δ and δ′ subunits and the χ and ψ subunits could not be resolved from each other because of the similarity in molecular masses between these subunits.

### Example 34

### Large Scale Growth of Clamp Loader Complex

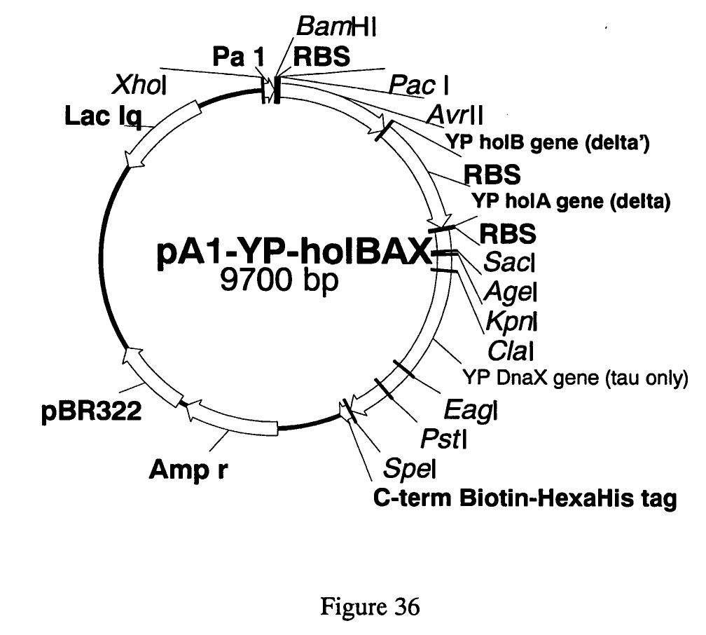

The large-scale growth of E. coli containing the pA1-YP-CL plasmid was as described in Example 4.4, except that in this large-scale growth to produce Y. pestis τ complex, the bacteria were allowed to continue growing for 3.5 h post-induction giving a harvest weight of 1.6 kg. Quality control results showed 10 out of 10 positive colonies on ampicillin-containing medium in the inoculum and 0 out of 10 positive colonies on ampicillin-containing medium at harvest. 70 g of a 1:1 suspension of frozen cells (35 g cells) in Tris-sucrose, which had been stored at −20° C. were lysed to determine the optimum concentration of ammonium sulfate with which to precipitate the largest amount of the core complex containing maximum activity but retaining the smallest amount of contaminating protein. This lysis was as described above in Example 4.5. Fr I was divided into seven samples of 15 ml each and labeled 20%, 30%, 40%, 50%, 60% and 70%. The protein in each sample was precipitated by adding varying amounts ammonium sulfate so that the final concentration of ammonium sulfate was: 20%, 30%, 40%, 50%, 60%, and 70% saturation, respectively, at 4° C. The mixture was stirred for an additional 30 min at 4° C. and the precipitate was collected by centrifugation (23,000×g, 45 min, 0° C.). The supernatant was removed from each sample and the resulting pellets were resuspended in buffer (25 mM Hepes (pH 7.5), 5 mM EDTA, 10% glycerol, 5 mM DTT, 100 mM NaCl) resulting in Fr II for all (6) ammonium sulfated precipitated samples. Protein concentrations of samples from both the supernatant and the different Fr II's are shown in FIG. 39.

### Example 35

### Purification of the Clamp Loader Complex

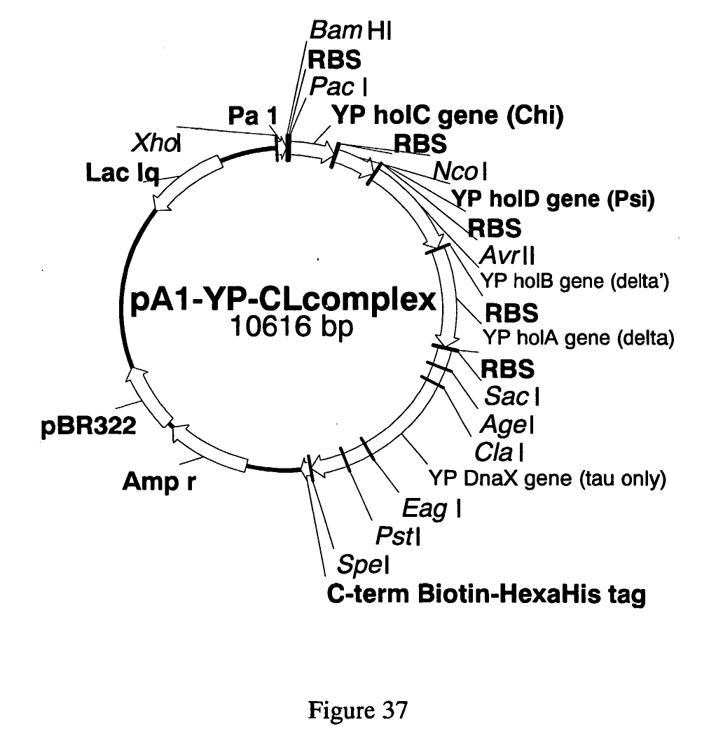

To determine if Y. pestis τ complex could stimulate E. coli core and β, assays were carried out in which Y. pestis τ complex from each of the ammonium sulfate precipitated protein Fr IIs was titrated into E. coli reconstitution assays as described in Example 4.6. The reconstitution assays (25 μl) contained 1 μl E. coli core (3×10⁶ units/mg, 4 mg/ml), 1 μl E. coli β (1.7×10⁶ units/mg, 1 mg/ml) and 19 μl of RNA primed M13Gori template. To this is added 2 μl of various dilutions of Fr II (AS precipitated protein) containing Y. pestis τ complex. The total amount of activity for each of the ammonium sulfate cuts is shown in FIG. 40. The Fr II from the 30% ammonium sulfate precipitation gave the greatest amount of activity with the lower concentration of contaminating protein and was use for purification of the τ complex.

To purify Y. pestis τ complex, 400 g of cells containing Y. pestis τ complex were lysed to form Fr I as described in Example 4.5 and resulted in 1650 ml of Fr I lysate. The protein in the sample was precipitated by gradually adding ammonium sulfate so that the final concentration of ammonium sulfate was 30% saturation at 4° C. The mixture was stirred for an additional 30 min at 4° C. and the precipitate was collected by centrifugation (23,000×g, 45 min, 0° C.). The protein pellet was resuspended in TG.51 buffer and diluted (480 ml, Fr II) to the conductivity of a DEAE chromatography column (300 ml, 5 cm×15 cm) equilibrated in TG.51/50 mM NaCl buffer. The sample was loaded onto the DEAE column (4.0 ml/min), washed with 5 column volumes of TG.51/50 mM NaCl buffer (5.0 ml/min) and eluted with 10 column volumes of TG.51 buffer containing a 50-400 mM NaCl gradient. Fractions (120) were collected containing 25 ml each. The protein eluted in two major peaks between fractions 6-20 and 30-50. The activity was contained in a single peak encompassed within the first protein peak in fractions 12-17 (140 ml). These fractions were pooled and SDS-polyacrylamide gel analysis indicated that the activity peak contain Y. pestis τ complex making up approximately 70% of the total protein. The protein in the sample was precipitated by ammonium sulfate so that the final concentration of ammonium sulfate was 60% saturation at 4° C. The mixture was stirred for an additional 30 min at 4° C. and the precipitate was collected by centrifugation (23,000×g, 45 min, 0° C.), quick frozen in liquid nitrogen and stored at −80° C.

The protein pellets containing Y. pestis τ complex were resuspended in imidazole buffer and homogenized using a Dounce homogenizer. The sample was clarified by centrifugation (16,000×g) and diluted (365 ml) using imidazole buffer minus NaCl to the conductivity of a hydroxylapatite chromatography column (150 ml, 5cm×9 cm) equilibrated in imidazole buffer plus 50 mM NaCl. This sample constituted Fr III. The sample was loaded onto the hydroxylapatite column (2.5 ml/min), washed with 2.5 column volumes of imidazole buffer/plus 10 mM KPO₄ and eluted with 10 column volumes of imidazole buffer containing a 10-150 KPO₄ gradient. Fractions (60) were collected containing 25 ml each. The protein eluted in a single peak between fractions 15 and 50. The activity eluted in a single peak within the broader protein peak. Fractions 36-45 were pooled (190 ml) and SDS-polyacrylamide gel electrophoresis indicated that the proteins of Y. pestis τ complex made up greater than 90% of the total protein in the pool. The protein in the sample was precipitated by ammonium sulfate so that the final concentration of ammonium sulfate was 50% saturation at 4° C. The mixture was stirred for an additional 30 min at 4° C. and the precipitate was collected by centrifugation (23,000×g, 45 min, 0° C.), quick frozen in liquid nitrogen and stored at −80°0 C.

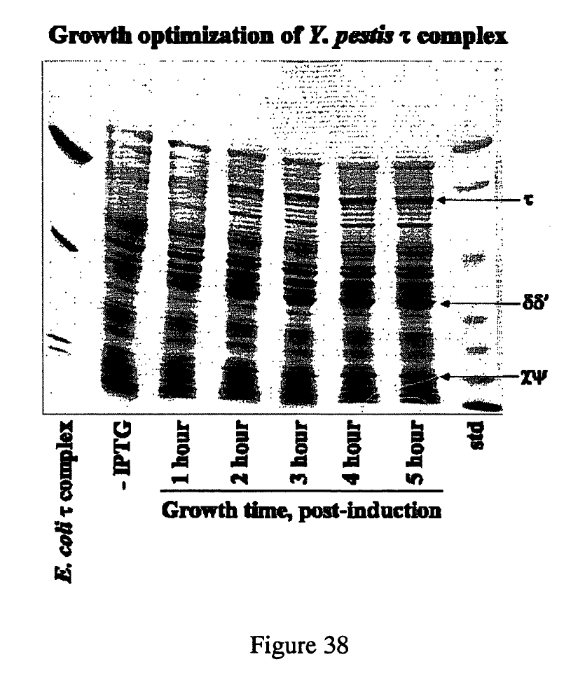

The protein pellets containing Y. pestis τ complex were resuspended in HG.05 buffer (25 mM HEPES, (pH 7.5), 50 mM KCl, 10% glycerol, 5 mM DTT and 1 mM EDTA) and homogenized using a Dounce homogenizer. The sample was clarified by centrifugation (16,000×g) and the resulting sample was Fr IV (7.0 ml). The sample was loaded onto a Sephacryl® S-200 (Amersham Pharmacia Biotech) chromatography column (250 ml, 1:20 ratio) equilibrated with HG.05 buffer. The procedure included running the buffer above the resin down to the resin bed, gently pipetting the sample onto the resin, pumping the sample into the resin, then 2 ml of buffer was gently added onto the top of the resin and pumped into the resin. The running buffer was then added to the top of the resin and elution was continued. The protein was eluted at a flow rate of 0.24 ml/min and 2.5 ml fractions were collected. Fractions 22-34 (32 ml) contained all of the loaded activity and were pooled (Fr V). Fr V was aliquoted and stored at −80° C. and represents the stock supply of Y. pestis τ complex. At each step of the purification process the purity of Y. pestis τ complex was analyzed by SDS polyacrylamide gel electrophoresis. A summary gel of the pooled protein for each purification step is shown in FIG. 41.

Densitometry scan of the protein bands in Fr V indicate that the ratio of τ, δ/δ′, and χ/ψ in the core complex is 3:1:1, respectively. This is indicative of three τ subunits, and one of each δ, δ′, χ and ψ subunits. The protein concentration and activity was also characterized at each purification step (Table IV).

TABLE IV

| | | | Protein | | Specific |
|---|---|---|---|---|---|
| | Volume | | concentration | Activity | Activity |
| Fraction | (ml) | Protein (mg) | (mg/ml) | (Units) | (Units/mg) |
| Fr I | 1650 | 30460 | 18.5 | — | — |
| Fr II | 480 | 2665 | 5.6 | 3.2 × 10⁹ | 1.2 × 10⁶ |
| Fr III | 365 | 1138 | 3.1 | 2.8 × 10¹⁰ | 2.5 × 10⁷ |
| Fr IV | 7 | 427 | 61.1 | 6.8 × 10¹⁰ | 1.6 × 10⁷ |
| Fr V | 32 | 352 | 11.0 | 2.2 × 10¹⁰ | 6.3 × 10⁷ |

### Example 36

### Optimization of Y. pestis Holoenzyme Components in Reconstitution Assays

To determine optimal concentration of the three components of the Y. pestis DNA polymerase III holoenzyme to be used in reconstitution assays for screening of chemical compound libraries, each component was titrated individually in assays while holding the other two components at constant concentrations. General procedures for the reconstitution assays are described in Example 4.5. In the first set of experiments the components held at constant concentrations were at concentration approximated to be saturating concentrations as determined from assays carried out during purification of the individual components. Saturating amounts of β₂, core (α, ε and θ) and τ complex (τ₃, δ, δ′, χ and ψ) were assumed to be 2.5, 0.5 and 0.7 pmol of component per reaction. The initial reaction showed that β₂, core and τ complex reached optimal activity at 0.03, 0.1 and 0.07 pmol, respectively.

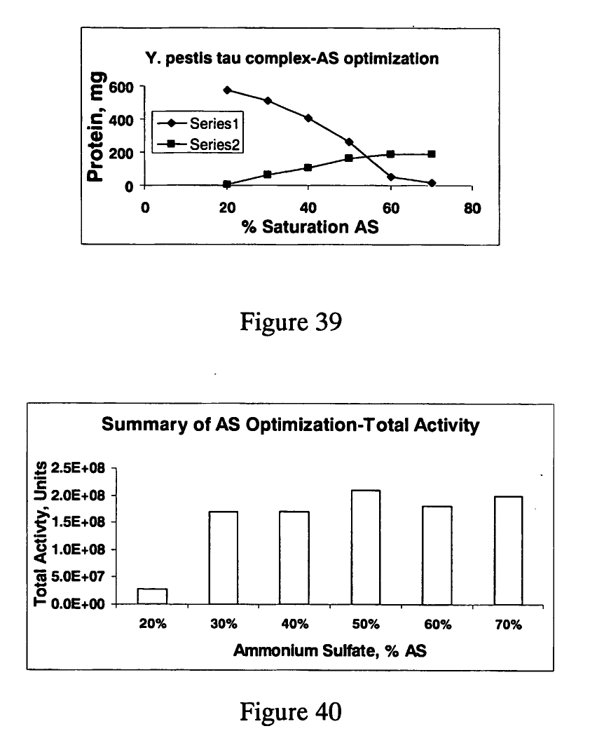

In the second set of experiments, the two components held constant, were at the concentration determined to have maximum activity in the first set of experiments and the third component was varied. In this set of experiments the lowest concentration giving maximum activity for β₂, core and τ complex was 0.03, 0.1 and 0.12 pmol, respectively. These concentrations for the three components in reconstitution assays are just above the linear response range (FIG. 42A-C). Therefore, any inhibition of activity of any of the three Y. pestis pol III holoenzyme components due to the influence of a screened chemical compound can be immediately detected.

Accessory Proteins

### Example 37

### Molecular Cloning of Y. pestis ssb, the Gene Encoding SSB Subunit Protein

The first project entailed PCRing the ssb gene (SSB) out of Y. pestis genomic DNA and inserting it into the vector pA1-CB-NcoI. The SSB subunit is composed of 183 amino acids. The gene encoding SSB is ssb, and is composed of 549 nts. The nucleotide sequence of ssb is represented by SEQ ID NO:53. The #4 codon, which codes for Arg, is a very low usage codon in E. coli. This was changed in the PCR reaction to a high usage codon. The sequence of the first four codons was changed from ATGGCCAGCAGA (SEQ ID NO:119) to ATGGCCAGCCGC (SEQ ID NO:120). The amino acid sequence of SSB is represented by SEQ ID NO:55. To PCR the ssb gene from Y. pestis genomic DNA, the forward/sense primer was: 5′-GATCccatggCCAGCCGCGGCGTAAACAAAGTGATTTTGG-3′ (40 mer) (SEQ ID NO:56). At the 5′ end is a clamp region to allow for efficient digestion with the restriction enzyme (upper case). Next, is an NcoI restriction site shown as lower case italic letters. The NcoI restriction site overlaps the first 4 nts of the 5′ end of the ssb gene (boldface letters). At codon #4 there is a very low usage Arg codon that was changed here. The first four codons (12 nts) are shown in boldface letters, and because of the modification of codon #4 do not correspond to the ssb gene. The next 22 nts that make up the sense strand (correspond) of the 5′ end of the ssb gene starting at codon #5 are shown as underlined capitals. The change at codon #4 creates a sequence beginning at position 14 that is GCCGCGGC and although capable of forming secondary structure, did not effect annealing of the primer to the substrate. The reverse/antisense primer was: 5′-GATCactagtttaTTAGAACGGGATGTCGTC-3′ (31 mer) (SEQ ID NO:57). At the 5′ end of the primer is a clamp region (GATC) to allow for efficient digestion with the restriction enzyme (uppercase letters). Next, is a SpeI restriction site shown as lowercase italic letters. This is followed by an additional (second) non-complementary stop codon (lowercase boldface letters). The second stop is adjacent to the native stop codon. The 18 nts complementary to 3′ end of the ssb gene (including the stop codon) are shown as uppercase underlined letters. The ssb gene was inserted into the pA1-CB-NcoI plasmid. The polyclonal region of pA1-CB-NcoI is shown in FIG. 31.

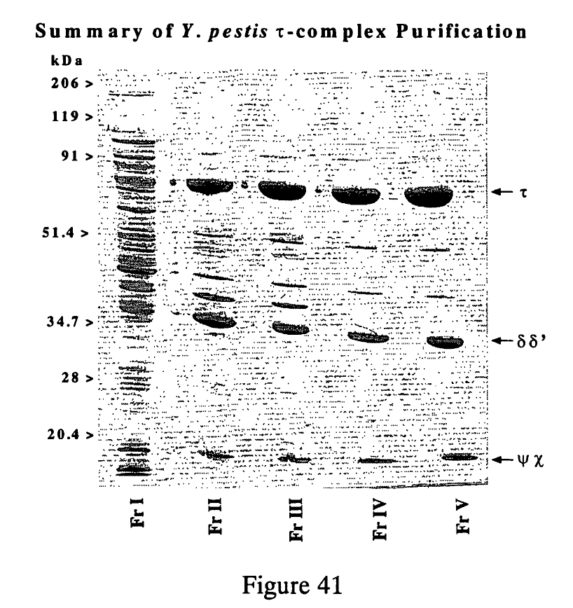

Both the PCR product and the plasmid pA1-CB-NcoI were digested with NcoI/SpeI restriction enzymes. The fragment of the PCR product containing the ssb gene was inserted into the digested pA1-CB-NcoI plasmid. This placed the ssb gene optimally spaced downstream of the RBS. This plasmid was named pA1-YP-ssb and is graphically shown in FIG. 43.

### 37.1. Expression of ssb

Construction of the plasmid shown in FIG. 43, pA1-YP-ssb has been completed. The correct sequences of all genes have been confirmed by DNA sequencing. The plasmid was transformed into AP1.L1 strain of E. coli and expressed as described in EXAMPLE 4.1. Mini-gels were used for analyses of expressed proteins as described in EXAMPLE 4.1, and was stained with Coomassie Blue. A protein band migrating at site expected for proteins with molecular mass corresponding to SSB (19.3 kDa) could be detected as distinct protein band, but was not observed in the uninduced control.

### 37.2. Large Scale Growth and Purification of Y. pestis SSB

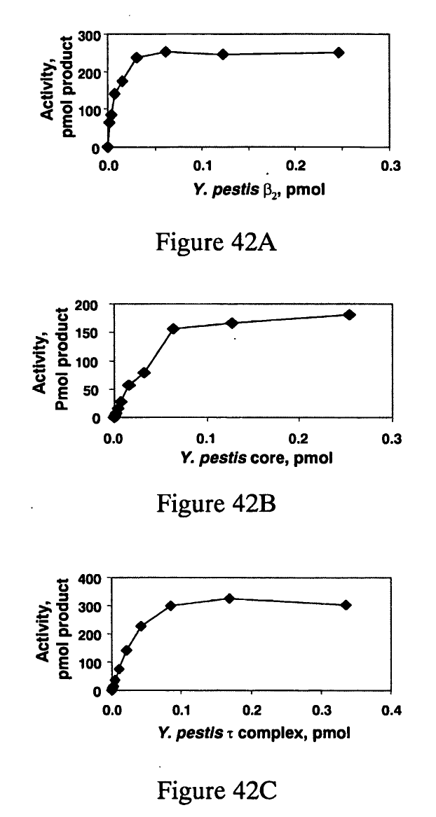

Time growth analysis for optimal expression of Y. pestis SSB was performed as described in EXAMPLE 10. The results of the growth optimization analyzed by SDS-polyacrylamide gel electrophoresis for E. coli containing pA1-YP-ssb is shown in FIG. 44. In these timed growth experiments, Y. pestis SSB was expressed at low levels.

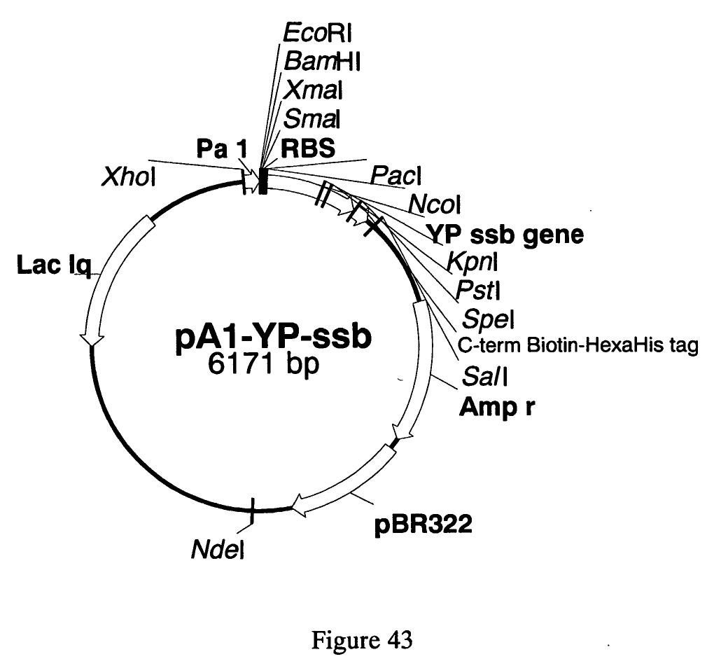

The large-scale growth of E. coli containing the pA1-YP-SSB plasmid was as described in Example 4.4. The exception was that in this large-scale growth to produce Y. pestis τ complex, the bacteria were allowed to continue growing for 3 h post-induction giving a harvest weight of 2.0 kg. Quality control results showed 10 out of 10 positive colonies on ampicillin-containing medium in the inoculum, 10 out of 10 positive colonies at induction and 10 out of 10 positive colonies on ampicillin-containing medium at harvest. 70 g of a 1:1 suspension of frozen cells (35 g cells) in Tris-sucrose, which had been stored at −20° C. were lysed to determine the optimum concentration of ammonium sulfate with which to precipitate the largest amount of the core complex containing maximum activity but retaining the smallest amount of contaminating protein. This lysis was as described in Example 4.5. Fr I was divided into seven samples of 15 ml each and labeled 30%, 35%, 40%, 45%, 50% and 55%. The protein in each sample was precipitated by adding varying amounts ammonium sulfate so that the final concentration of ammonium sulfate was: 30%, 35%, 40%, 45%, 50%, and 55% saturation, respectively, at 4° C. The mixture was stirred for an additional 30 min at 4° C. and the precipitate was collected by centrifugation (23,000×g, 45 min, 0° C.). The supernatant was removed from each sample and the resulting pellets were resuspended in buffer (25 mM Hepes (pH 7.5), 1 mM EDTA, 10% glycerol, 5 mM DTT, 100 mM NaCl) resulting in a Fr II for all (6) ammonium sulfated precipitated samples. Protein concentrations of samples from both the supernatant and the different Fr II's are shown in FIG. 45.

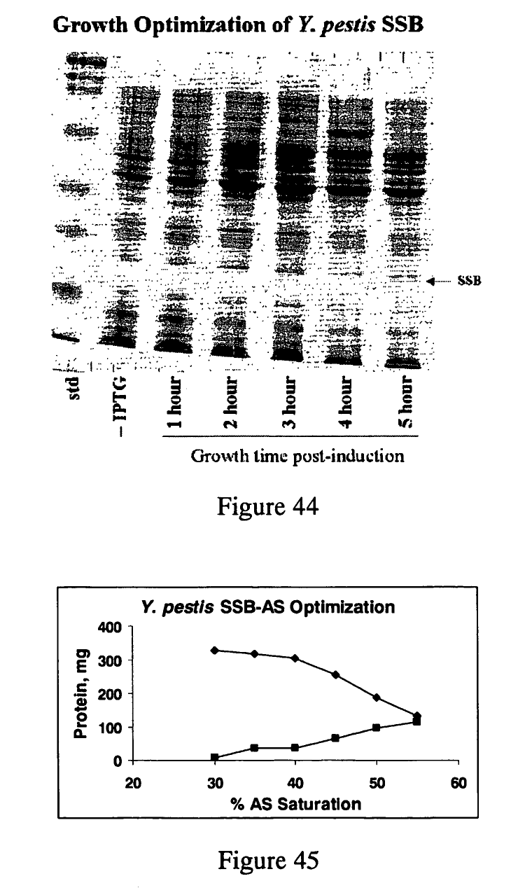

The samples containing the resuspended protein pellets (Fr IIs) were also analyzed by SDS polyacrylamide gel electorphoresis and the results are shown in FIG. 46.

To purify Y. pestis SSB 600 g of cells containing Y. pestis SSB was lysed to form Fr I as described in Example 4.5 and resulted in 2380 ml of Fr I lysate. The protein in the sample was precipitated by gradually adding ammonium sulfate so that the final concentration of ammonium sulfate was 30% saturation at 4° C. The mixture was stirred for an additional 30 min at 4° C. and the precipitate was collected by centrifugation (23,000×g, 45 min, 0° C.). The protein pellets were resuspended in TG.52 buffer (50 mM Tris-HCl, (pH 7.5), 20% glycerol, 1 mM EDTA, 5 mM DTT) and diluted (100 ml, Fr II) to the conductivity of a Blue Dextran-Sepharose chromatography column (800 ml, 5 cm×38 cm) equilibrated in TG.52/100 mM NaCl buffer. The sample was loaded onto the blue dextran-sepharose column (1 ml/min), and eluted in a stepwise fashion by washing the column with TG.52 buffer containing increasing concentrations of NaCl. The column was initially washed (5 ml/min) with 1 column volume of TG.52/0.1 M NaCl buffer. Second the column was washed (5 ml/min) with 3 column volumes of TG.52/0.5 M NaCl buffer. Third, the column was washed (1.5 ml/min) with 2 column volumes of TG.52/1 M NaCl buffer. At this point 25 ml fractions (130 total) were collected through final elution step. The column was next washed (5 ml/min) with 1 column volume of TG.52/2 M NaCl buffer. Finally the column was washed with 3 column volumes of TG.52/4 M NaCl buffer. The protein eluted in numerous peaks across all washes. The Y. pestis SSB eluted in a single peak encompassed within fractions 85-130 (Fr III, 1050 ml). SDS-polyacrylamide gel analysis indicated that Y. pestis SSB was greater than 95% homogeneous.

The Y. pestis SSB was loaded (1.5 ml/min) directly onto a hydroxylapatite column (40 ml, 2.5×8 cm) equilibrated in imidazole buffer (50 mM imidazole, (pH 6.8), 10% glycerol, 50 mM NaCl, 5.0 mM DTT), washed (2 ml/min) with 2 column volumes of imidazole buffer and eluted with 5 column volumes of imidazole buffer containing 70 mM KPO₄. Fractions (34) were collected containing 3 ml each. The protein eluted in a single peak between fractions 11-22 (Fr IV, 36 ml). The protein in the sample was precipitated by ammonium sulfate so that the final concentration of ammonium sulfate was 50% saturation at 4° C. The mixture was stirred for an additional 30 min at 4° C. and the precipitate was collected by centrifugation (23,000×g, 45 min, 0° C.), quick frozen in liquid nitrogen and stored at −80° C.

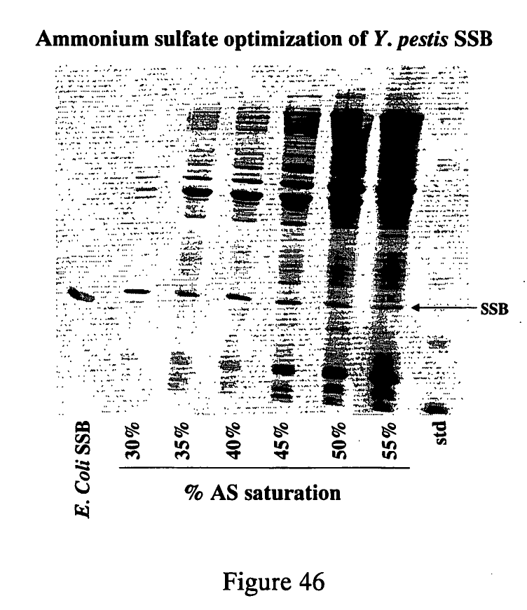

The protein pellets were resuspended in 3 ml of TG.52 buffer and loaded onto a Sephacryle S-200 (Amersham Pharmacia Biotech) chromatography column (250 ml, 1:25 ratio) equilibrated with HG.05 buffer. The procedure included running the buffer above the resin down to the resin bed, gently pipetted the sample onto the resin, pumping the sample into the resin, then 2 ml of buffer was gently added onto the top of the resin and pumped into the resin. The running buffer was then added to the top of the resin and elution was continued. The protein was eluted at a flow rate of 0.2 ml/min and 1 ml fractions (100) were collected. Fractions 47-57 (11 ml) were pooled (Fr V). Fr V was aliquoted and stored at −80° C. and represents the stock supply of Y. pestis SSB. At each step of the purification process the purity of Y. pestis SSB was analyzed by SDS polyacrylamide gel electrophoresis. A summary gel of the pooled protein for each purification step is shown in FIG. 47.

The protein concentration was also characterized at each purification step (Table V).

TABLE V

| | Protein | 37.3. Volume | Concentration |
|---|---|---|---|
| Y. pestis SSB | mg | ml | mg/ml |
| Fr I | 40,000 | 2380 | 17 |
| Fr II | 810 | 100 | 8 |
| Fr III | 150 | 1050 | 0.15 |
| Fr IV | 120 | 36 | 3.3 |
| Fr V | 120 | 11 | 11 |

### Example 38

### Molecular Cloning of dnaG, the Gene Encoding DnaG (Primase)

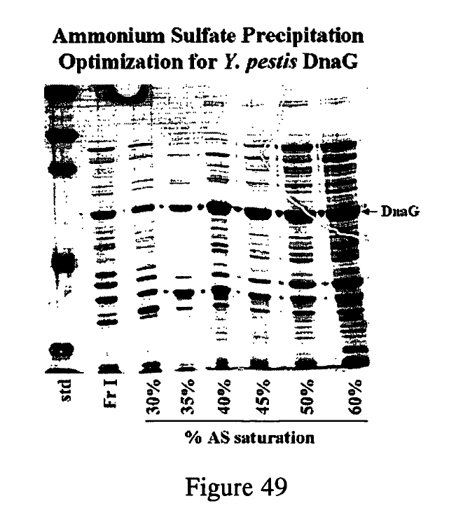

The second project entailed amplification of the dnaG gene (encoding DnaG primase) from the Y. pestis genomic DNA by PCR and inserting it into the vector pA1-CB-NcoI. The DnaG subunit is composed of 583 amino acids. The dnaG gene is composed of 1749 nts. The nt sequence of dnaG is represented by SEQ ID NO:58. Codons #3 and 4 are low usage codons in E. coli and were changed to high usage codons in the PCR reaction. The native sequence of the first 4 codons is ATGGCTGGACGA (SEQ ID NO:61), this was changed to ATGGCTGGTCGT (SEQ ID NO:62)in the PCR reaction. The modified codons are underlined. The amino acid sequence of DnaG is represented by SEQ ID NO:60. The forward/sense primer in the PCR reaction was: 5′-GATCccATGGCTGGTCGTATTCCACGTGTATTTATCAATG-3′ (40 mer) (SEQ ID NO:63). At the 5′ end of the primer is a clamp region (GATC) to allow for efficient digestion with the restriction enzyme (uppercase letters). Next, is an NcoI restrictions site that is shown as underlined italic letters. The first four nts of the 5′ end of the dnaG gene, starting with the ATG start codon overlaps the NcoI restriction site. The first four codons of the 5′ end of the dnaG gene, which contain the modified codons #3 and 4, are shown as capitalized italic letters. The 22 nts that correspond to the 5′ end of dnaG beginning at codon #5 are shown in underlined capital letters. The reverse/antisense primer was: 5′-GATCactagttcaTCATTTTTTTCTGGCTAGTG-3′ (33 mer) (SEQ ID NO:64). At the 5′ end of the primer is a clamp region (GATC) to allow for efficient digestion with the restriction enzyme (upper case). Following this is a SpeI restriction site, which is shown as lowercase italic letters. Next, is an additional stop codon (lower case, boldface letters), which is adjacent to the native stop codon. Finally, the 20 nts complementary to the 3′ end of dnaG including the native stop codon is shown as underlined uppercase letters. Both the PCR product and the plasmid pA1-CB-NcoI were digested with NcoI/SpeI restriction enzymes. The fragment of the PCR product containing the dnaG gene was inserted into the digested pA1-CB-NcoI plasmid. This placed the dnaG gene optimally spaced downstream of the RBS. This plasmid was named pA1-YP-dnaG and is graphically shown in FIG. 50.

### 38.1. Expression of DnaG

Construction of the plasmids shown in FIG. 50, pA1-YP-dnaG, has been completed. The correct sequences of all genes have been confirmed by DNA sequencing.

The plasmids was transformed into AP1.L1 strain of E. coli and expressed as described above in Example 4.1. Cellular protein was analyzed and a protein band migrating at the site expected for proteins with molecular mass corresponding to DnaG (65.6 kDa) could be detected as distinct protein band, but was not observed in the uninduced control.

### 38.2. Large Scale Growth and Purification of Y. pestis DnaG

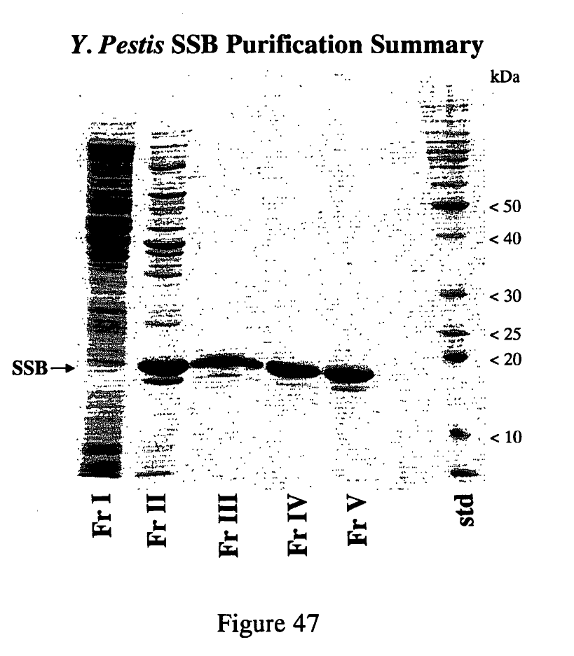

Time growth analysis for optimal expression of Y. pestis DnaG was as described in EXAMPLE 10. The results of the growth optimization analyzed by SDS-polyacrylamide gel electrophoresis for E. coli containing pA1-YP-dnaG is shown in FIG. 48. In this gel, Y. pestis DnaG was expressed at approximately 5% of the total cellular proteins.

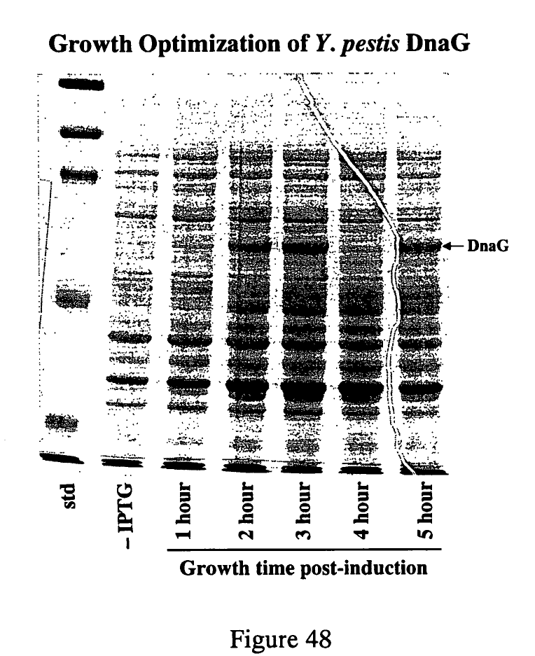

The large-scale growth of E. coli containing the pA1-YP-dnaG plasmid was as described in Example 4.4, exception was that in this large-scale growth to produce Y. pestis DnaG, the bacteria were allowed to continue growing for 3 h post-induction giving a harvest weight of 2.0 kg. Quality control results showed 10 out of 10 positive colonies on ampicillin-containing medium in the inoculum, 10 out of 10 positive colonies at induction and 9 out of 10 positive colonies on ampicillin-containing medium at harvest. 60 g of a 1:1 suspension of frozen cells (30 g cells) in Tris-sucrose, which had been stored at −20° C. were lysed to determine the optimum concentration of ammonium sulfate with which to precipitate the largest amount of the core complex containing maximum activity but retaining the smallest amount of contaminating protein. This lysis was as described in Example 4.5. Fr I was divided into six samples of 20 ml each and labeled 30%, 35%, 40%, 45%, 50% and 60%. The protein in each sample was precipitated by adding varying amounts ammonium sulfate so that the final concentration of ammonium sulfate was: 30%, 35%, 40%, 45%, 50% and 60% saturation, respectively, at 4° C. The mixture was stirred for an additional 30 min at 4° C. and the precipitate was collected by centrifugation (23,000×g, 45 min, 0° C.). The supernatant was removed from each sample and the resulting pellets were resuspended in TG.52 buffer (50 mM Tris-HCl (pH 7.5), 1 mM EDTA, 20% glycerol, 5 mM DTT) resulting in Fr II for all (5) ammonium sulfated precipitated samples. SDS-polyacrylamide gel analysis of the different Fr II's are shown in FIG. 49.

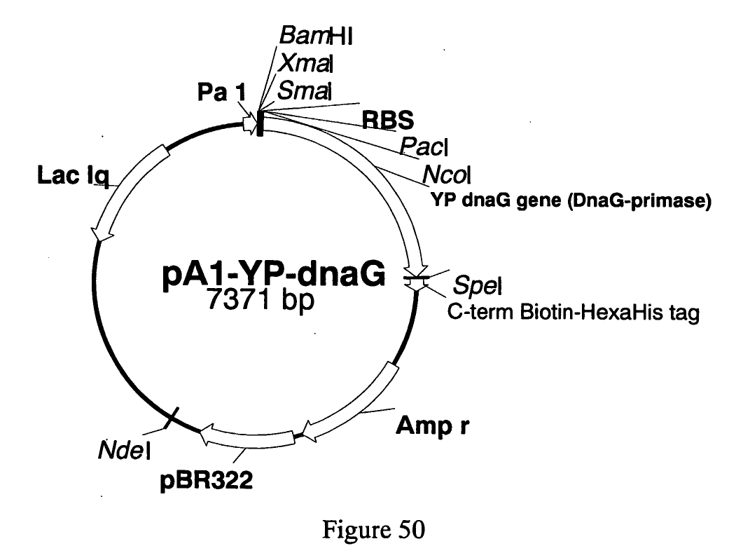

To determine if Y. pestis DnaG could function in reconstitution assays composed of E. coli DNA polymerase III holoenzyme reconstitution assays (described in Example 4.6), and to determine optimal ammonium sulfate concentrations to use in purifying Y. pestis DnaG, 2 μl of various dilution of Fr II from the different ammonium sulfate precipitated samples were tested in reconstitution assays. Reconstitution assays (25 μl) contained 1 μl E. coli core (3×10⁶ units/mg, 4 mg/ml) diluted 1:50, 1 μl E. coli β (1.7×10⁶ units/mg, 1 mg/ml) diluted 1:50, 2 μl τ complex (2.8×10⁶ units/mg, 2 mg/ml) diluted 1:100 and 19 μl of RNA primed M13Gori template. The results shown in FIG. 51 indicate that Y. pestis DnaG can indeed function with the other components from E. coli holoenzyme. These assays indicate that maximum amounts of Y. pestis DnaG containing minimal amounts of contaminating proteins precipitate at 45% ammonium sulfate.

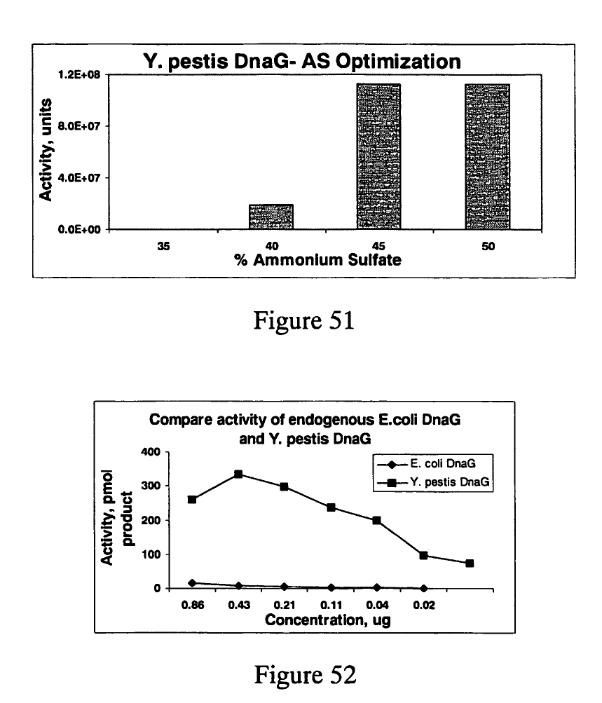

To determine the effect if any of endogenous E. coli DnaG, we carried out parallel experiments in which samples from the 45% ammonium sulfate precipitated Y. pestis DnaG and samples from the preparation of 45% ammonium sulfate precipitated Y. pestis SSB described above were titrated into reconstitution assays. These experiments indicate that endogenous E. coli DnaG does not contribute to activity of the 45% ammonium sulfate precipitated Y. pestis DnaG as shown in FIG. 52).

To purify Y. pestis DnaG 400 g of cells containing Y. pestis DnaG were lysed to form Fr I as described in Example 4.5 and resulted in 1400 ml of Fr I lysate. The protein in the sample was precipitated by gradually adding ammonium sulfate so that the final concentration of ammonium sulfate was 45% saturation at 4° C. The mixture was stirred for an additional 30 min at 4° C. and the precipitate was collected by centrifugation (23,000×g, 45 min, 0° C.). One-half of the protein pellets were resuspended in TG.51 buffer and diluted to the conductivity of a P-11 phosphocellulose (Whatman) chromatography column (500 ml, 6 cm×26 cm) equilibrated in TG.51/50 mM NaCl buffer. The sample was loaded onto the P-11 phosphocellulose column (4 ml/min), washed with 1 column volumes of TG.51/50 mM NaCl buffer and eluted with 10 column volumes of TG.51 buffer containing a 50-450 mM NaCl gradient. Fractions (150) were collected containing 25 ml each. The protein eluted across the second two-thirds of the gradient. The activity eluted in a peak between fractions 50-80. Fractions 60-79 were pooled (500 ml) and contained approximately 20% of the total activity loaded onto the column. SDS-polyacrylamide gel electrophoresis analysis of the fractions indicated that Y. pestis DnaG made up over 90% of the total protein. The protein in the sample was precipitated by ammonium sulfate so that the final concentration of ammonium sulfate was 50% saturation at 4° C. The mixture was stirred for an additional 30 min at 4° C. and the precipitate was collected by centrifugation (23,000×g, 45 min, 0° C.), quick frozen in liquid nitrogen and stored at −80° C. This entire process was then repeated with the second half of the protein pellets from ammonium sulfate precipitating the protein from FrI.

The protein pellets containing Y. pestis DnaG were resuspended in imidazole buffer (50 mM imidazole, (pH 6.8), 10% glycerol, 50 mM NaCl, 5.0 mM DTT) and homogenized using a Dounce homogenizer. The sample was clarified by centrifugation (16,000×g) and diluted (FrIII, 920 ml) using imidazole buffer minus NaCl to the conductivity of a hydroxylapatite chromatography column (500 ml, 5cm×25 cm) equilibrated in imidazole buffer. The sample was loaded onto the hydroxylapatite column, washed with 3 column volumes of imidazole buffer/plus 10 mM KPO₄ and eluted with 10 column volumes of Imidazole buffer containing a 10-225 KPO₄ gradient. Fractions (200) were collected containing 25 ml each. The protein and the activity eluted in a single peak between fractions 60-110. Fractions 68-88 were pooled (540 ml) and contained 30% of the total activity loaded onto the column. The protein in the sample was precipitated by ammonium sulfate so that the final concentration of ammonium sulfate was 55% saturation at 4° C. The mixture was stirred for an additional 30 min at 4° C. and the precipitate was collected by centrifugation (23,000×g, 45 min, 0° C.), quick frozen in liquid nitrogen and stored at −80° C.

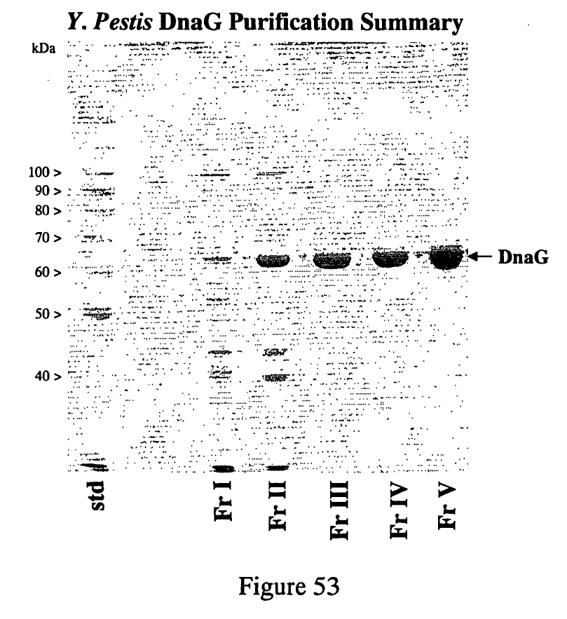

The protein pellets containing Y. pestis DnaG was resuspended in HG.05 buffer (25 mM HEPES, (pH 7.5), 50 mM KCl, 10% glycerol, 5 mM DTT and 1 mM EDTA) and homogenized using a Dounce homogenizer. The sample was clarified by centrifugation (16,000×g) and the resulting sample was Fr IV (7 ml, 39 mg/ml). The sample was loaded onto a Sephacryl® S-200 (Amersham Pharmacia Biotech) chromatography column (250 ml, 1:20 ratio) equilibrated with HG.05 buffer. The procedure included running the buffer above the resin down to the resin bed, gently pipetted the sample onto the resin, pumping the sample into the resin, then 2 ml of buffer was gently added onto the top of the resin and pumped into the resin. The running buffer was then added to the top of the resin and elution was continued. The protein was eluted at a flow rate of 0.2 ml/min and 2 ml fractions were collected. The protein eluted in two peaks, the first containing contaminating protein and the second larger peak containing Y. pestis DnaG. Fractions 38-46 (20 ml) were pooled (Fr V, 14 mg/ml). Fr V was aliquoted and stored at −80° C. and represents the stock supply of Y. pestis DnaG. At each step of the purification process the purity of Y. pestis DnaG was analyzed by SDS polyacrylamide gel electrophoresis. A summary gel of the pooled protein for each purification step is shown in FIG. 53.

The protein concentration and activity was also characterized at each purification step

TABLE VI

| | | | Protein | | Specific |
|---|---|---|---|---|---|
| | Volume | | concentration | Activity | Activity |
| Fraction | (ml) | Protein (mg) | (mg/ml) | (Units) | (Units/mg) |
| Fr I | 1400 | 31302 | 22.5 | 3.6 × 10⁹ | 1.0 × 10⁵ |
| Fr II* | 1260 | 7461 | 10.8 | 2.8 × 10¹⁰ | 3.8 × 10⁶ |
| Fr III | 920 | 1150 | 1.3 | 8.8 × 10⁸ | 0.8 × 10⁶ |
| Fr IV | 7 | 274 | 39 | 2.1 × 10⁸ | 0.7 × 10⁶ |
| Fr V | 20 | 131 | 6.6 | 7.6 × 10⁸ | 5.8 × 10⁶ |

*One-half of FrII was prepared for each P-11 phosphocellulose column run. Both FrII preparations and both FrIII pools were combined to give the numbers shown in this table.

Reconstitution

### Example 39

### Reconsitution of Y. pestis Holoenzyme Including SSB and DnaG

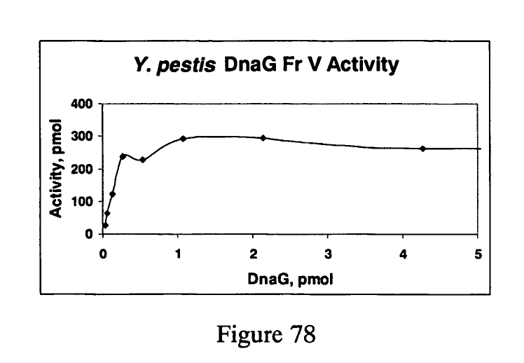

The activity of DnaG was determined using a modified reconstitution assay. In this assay 19 μl containing 0.06 pmol M13Gori DNA (ca. 500 pmol total nucleotide), 1.6 μg SSB, 10 mM magnesium acetate, 200 μM ATP, GTP, CTP and UTP, 48 μM dATP, dGTP, dCTP and 18 μM [³H]dTTP (100 cpm/pmol) were mixed with 4 μl containing 1000 units of Y. pestis DnaE, 14000 units of Y. pestis clamp-loading complex and 12000 units of β. The reaction was initiated by adding 2 μl of various dilutions of Y. pestis DnaG. The results of this are shown in FIG. 78.

### Example 40

### Complete Replicative Polymerase for Y. pestis

With a functional replicative polymerase from Y. pestis in hand, libraries of small molecules (obtained from several suppliers of combinatorial chemistry libraries) will be screened in a high-throughput format to identify inhibitors of the Y. pestis replicative polymerase. The initial target of drug screening will minimally include the components assembled as described above (pol III core, clamp-loader complex, beta, SSB and DnaG). Additional components of Y. pestis replicative polymerase (such as DnaB helicase) will be added to the screen as they become available. With replication systems from multiple pathogens in hand, a method of identifying both narrow- and broad-spectrum antibacterial drugs will comprise setting up parallel screens against replication systems from multiple organisms. Importantly, since the replication system represents a novel target for antibacterial drugs, resistance mechanisms should not yet exist.

### Example 41

### DNA Replication Multiplicative Target Screens™ for Use in Screening for New Antibacterial Drugs

The process of DNA replication is central to the propagation of all bacteria. To date, none of the antibacterials that are commercially available target any of the enzymes that make up the central replication system in bacteria. This is startling, considering that DNA replication is among the most essential of cellular processes. The DNA replication machinery of infectious organisms therefore stands as an unexplored target for drug development efforts and presents a significant opportunity. A replication system has not previously been targeted in large part because its complexity poses a formidable technological barrier to setting up drug screening assays. In addition, since activity of many individual subunits depends on proper association with other components of the replication apparatus, target-based assays using single subunits are generally not feasible. A substantial portion of the system must therefore be reconstituted for drug screening.

The DNA polymerase III holoenzyme of bacteria contains approximately 10 subunits that undergo marked changes in protein-protein association during each of the steps of the complex reaction they catalyze. Since all of these proteins must function together, the multitude of distinct interactions and catalytic events all provide targets for antibacterial action and thus provide a multiple of the proteins present as targets. Simple fluorescence-based assays in a high-throughput screening format have been developed that provide the basis for what is termed herein the Multiplicative Target Screening™ (MTS™) technology. This technology also included efficient high-throughput approaches for deconvolution and efficient identification of the specific target. Assays utilizing the MTS technology are profoundly more efficient compared to the more traditional screening assays using single target enzymes since activities of each of the proteins that comprise the holoenzyme are targeted simultaneously.

Parallel target screens can be used for the identification of hits with broad-spectrum and narrow-spectrum specificity. An E. coli MTS™ has been developed and will be used in the process of reconstituting other Gram-negative and Gram-positive MTS™ systems. These assays, together with a reconstituted human replicase will be used to identify ‘hits’ with broad and narrow spectrum potential and to eliminate compounds with toxic potential that inhibit the human (DNA polymerase δ, PCNA, RFC, RPA) replication system. For example, a compound that inhibits a Gram-negative and Gram-positive replicase without inhibiting the human replicase provides a potential candidate that can be developed into a broad-spectrum antibacterial. This strategy can be reversed (i.e., identify a compound that inhibits one Gram-negative replicase without inhibiting a different Gram-negative replicase) to obtain antibacterials that are useful for treatment of specific chronic infections where the ideal antibacterial drug would be fairly specific and not lead to the elimination of normal, nonpathogenic flora. Compounds that specifically inhibit the P. aeruginosa replication apparatus should be useful as chronically administered drugs to benefit cystic fibrosis and immuno-compromised patients.

### Example relating to P. aeruginosa Nucleic Acids and Proteins

Pol III Core

### Example 42

### Molecular Cloning of P. aeruginosa dnaE, the Gene Encoding the dnaE (Alpha) Subunit

The recently completed P. aeruginosa genome database (Stover, et al. ibid.) has been searched for genes that encode the DNA polymerase III holoenzyme subunits. The gene for dnaE (α) subunit has been identified and annotated. Comparison of amino acid sequences for DnaE proteins from P. aeruginosa and E. coli shows that they are 58% identical and align over the entire length of the protein sequence (FIG. 3). Two dnaE candidates have been identified and both are cloned and inserted into core operons. The first dnaE gene (dnaE #1) is described first.

### 42.1. Molecular Cloning of Candidate #1

The dnaE #1 gene, which encodes the a #1 subunit, is amplified from P. aeruginosa genomic DNA. The dnaE #1 gene is then inserted into the pA1-CB-NcoI plasmid (polyclonal region of pA1-CB-NcoI is shown in FIG. 9). The dnaE #1 gene does not have any low-usage codons for expression in E. coli. This procedure is a two-step process. The 5′ one-half of dnaE #1 is first PCR amplified and inserted into pA1-CB-NcoI thereby forming pA1-PA-dnaE#1-5′. The 3′ one-half of dnaE#1 is then PCR amplified and inserted into pA1-PA-dnaE#1-5′, which creates pA1-PA-core#1. The a #1 subunit is composed of 1173 amino acids. The gene-encoding α #1 is dnaE #1, and is composed of 3522 nucleotides including the stop codon. The nucleotide sequence of dnaE #1 is represented by SEQ ID NO:65. The amino acid sequence of α #1 is represented by SEQ ID NO:67. To PCR amplify the 5′ one-half of dnaE #1, the forward/sense primer is: 5′-gactTTAATTAAGCTTATGACCGTATCCTTCGTTCA-3′ (36 mer) (SEQ ID NO:68). There is a 4-nucleotide 5′ clamp (lowercase gact) allowing efficient digestion by the restriction enzyme. The clamp region is followed by a PacI site (uppercase, italic letters), which is used for insertion into pA1-CB-NcoI. The PacI site overlaps a HindIII restriction site (continued uppercase, italic letters), which is used in operon construction. This spaces the start codon 12 nucleotides downstream from the RBS, which is just outside the optimal spacing range of 9-11 nucleotides. The dnaE #1 gene is designed to be part of the core operon, but can express alone in this plasmid with spacing from the RBS just above optimal. Finally, the 20 nucleotides corresponding to the first 20 nucleotides at the 5′ end of the dnaE #1 gene are shown as upper-case and underlined.

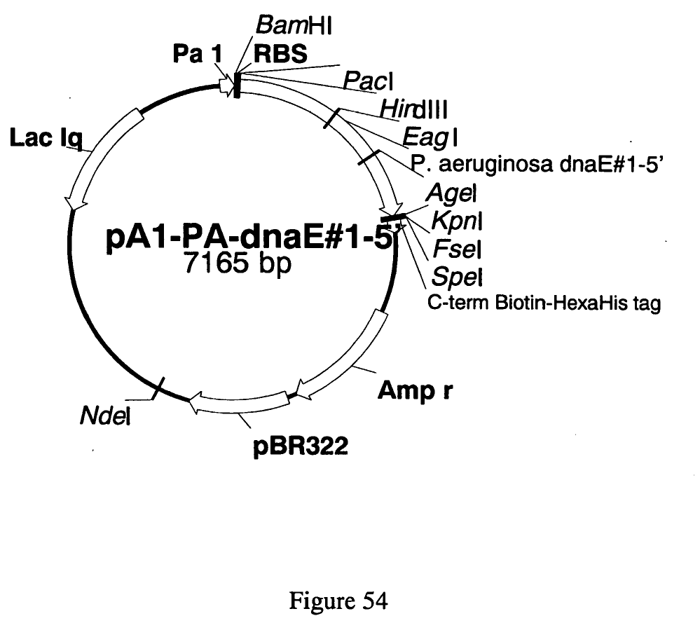

There is a unique KpnI restriction site located approximately 1527 nucleotides downstream of the dnaE #1 start codon. A reverse/antisense primer is selected just downstream of this unique KpnI restrictions site. The 1.5 kilobase (kb) PCR product contains the 5′ half of the dnaE #1 gene. The PCR product is digested with PacI/KpnI restriction enzymes. The PCR fragment (approximately 1.5 kb) is then inserted into pA1-CB-NcoI that has been digested with the same two restriction enzymes. This precursor plasmid is named pA1-PA-dnaE#1-5′ and is graphically shown in FIG. 54.

To PCR amplify the 3′ one-half of the dnaE #1 gene; a forward/sense primer is selected from the sequence just upstream of the unique KpnI restriction site. This allows digestion of the PCR product with KpnI and insertion of it at the KpnI site of pA1-PA-dnaE#1-5′ thereby reforming the entire dnaE #1 gene. The reverse/antisense primer is: 5′-agtcACTAGTtaTTAACGATAATTGAGGAAGA-3′ (32 mer) (SEQ ID NO:69). There is a 4-nucleotide 5′ clamp (lowercase agtc) to allow efficient digestion by the restriction enzyme. A SpeI restriction site (uppercase, italic letters) follows the clamp. The SpeI restriction site overlaps the “t” of the second non-complementary stop codon (bold). The second non-complementary stop codon is adjacent to the native stop and is in tandem with the native stop codon. The 20 nucleotides which are complementary to the 3′ 20 nucleotides of the dnaE #1 gene are shown as upper case and underlined. The 1.5 kb PCR product contains the 3′ half of the dnaE #1 gene.

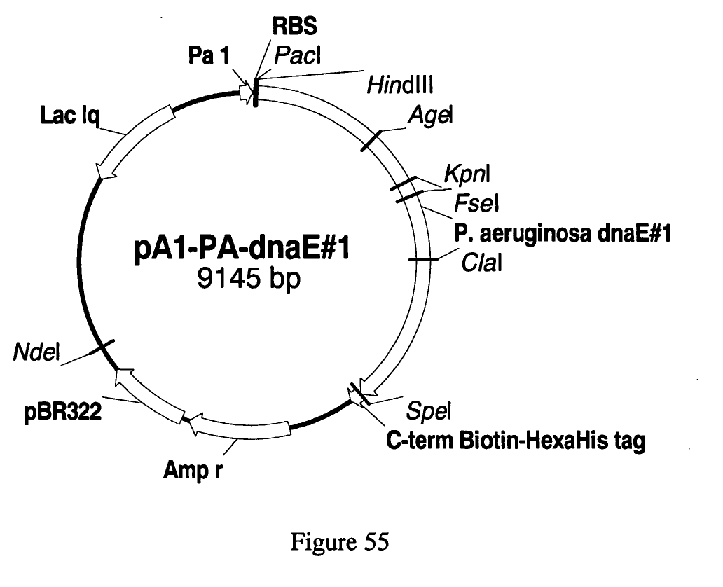

The PCR product is digested with KpnI/SpeI and inserted into pA1-PA-dnaE#1-5′ that has been digested with the same two restriction enzymes. This places the entire dnaE #1 gene in the plasmid with everything in the correct reading frame. This plasmid is named pA1-PA-dnaE#1 and is graphically shown in FIG. 55.

### 42.2. Molecular Cloning of Candidate #2

Here, the dnaE #2 gene, which encodes the a #2 subunit, is PCR amplified from P. aeruginosa genomic DNA. The dnaE #2 gene is inserted into the pA1-CB-NcoI plasmid (polyclonal region of pA1-CB-NcoI is shown above). The dnaE #2 gene has only one low usage codon, which is the first codon, for expression in E. coli. In P. aeruginosa dnaE #2 the start codon is GTG. This is changed to ATG by the forward/sense primer. This procedure will be a two-step process. First, the 5′ one-third of dnaE #2 is PCR amplified and inserted into pA1-CB-NcoI thereby forming pA1-PA-dnaE#2-5′. Second, the 3′ two-thirds of dnaE #2 is then PCR amplified and inserted into pA1-PA-dnaE#2-5′, which will create a plasmid containing the full-length dnaE #2 gene. The a #2 subunit is composed of 1031 amino acids. The gene encoding a #2 is dnaE #2, and is composed of 3096 nucleotides including the stop codon. The nucleotide sequence of dnaE #2 is represented by SEQ ID NO:70. The first codon that codes for Met is GTG. This is changed to ATG by the forward/sense primer. The amino acid sequence of a #2 is represented by SEQ ID NO:72. To PCR amplify the 5′ one-third of dnaE #2, the forward/sense primer is: 5′-gctaTTAATTAAGCTTATGGCTGCATGGCTGGTTCGCAT-3′ (39 mer) (SEQ ID NO:73. There is a 4-nucleotide 5′ clamp (lower case-gcta) allowing efficient digestion by the restriction enzyme. The 4-nucleotide clamp is followed by a PacI restriction site (uppercase, italic letters), which is used for insertion into pA1-CB-NcoI. The PacI restriction site overlaps the first two nucleotides of a HindIII restriction site (also upper-case, italic letters). The HindIII restriction site will be used in operon construction. The restriction sites are followed by a modified start codon (from GTG>ATG), shown in uppercase bold letters. The 20 nucleotides corresponding to the 5′ end of dnaE#2 at positions 4-23 are shown as uppercase underlined letters.

There is a unique KpnI site approximately 800 nucleotides downstream of the dnaE #2 gene start codon. A primer is selected just downstream of this KpnI restriction site to serve as a reverse/antisense primer. The 0.8 kb PCR product contains the 5′ one-third of the dnaE #2 gene.

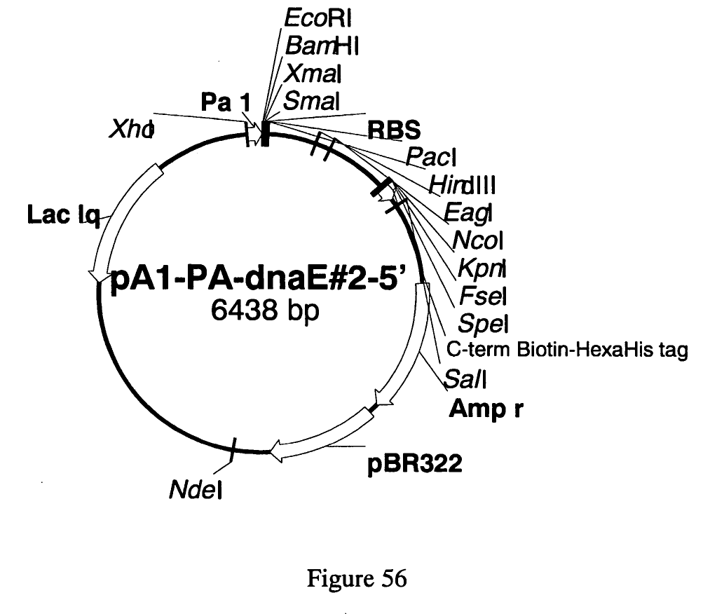

This PCR product is digested with PacI/KpnI restriction enzymes. The PCR fragment (approximately 0.8 kb) is then inserted into pA1-CB-NcoI that has been digested with the same two restriction enzymes. This precursor plasmid is named pA1-PA-dnaE#2-5′ and is graphically shown in FIG. 56. To PCR amplify the 3′ two-thirds of the dnaE #2 gene; a forward/sense primer is selected from the sequence just upstream of the unique KpnI restriction site. This allows digestion of the PCR product with KpnI and insertion of it at the KpnI site of pA1-PA-dnaE#2-5′ thereby reforming the entire dnaE #2 gene. The reverse/antisense primer is: 5′-agtcACTAGTcaTCAATGGAAATCCCGGCTGCGGA-3′ (35 mer) (SEQ ID NO:74). There is a 4-nucleotide 5′ clamp (lower case-agtc) allowing efficient digestion by the restriction enzyme. A SpeI restriction site (upper-case, italic letters) follows the clamp. The SpeI restriction site overlaps the “t” of the second non-complementary stop codon (bold). The second non-complementary stop codon is adjacent to the native stop and is in tandem with the native stop codon. The 20 nucleotides that are complementary to the 3′ 23 nucleotides of the dnaE #2 gene are shown as upper case and underlined. The 2.2 kb PCR product contains the 3′ two-thirds of the dnaE #2 gene.

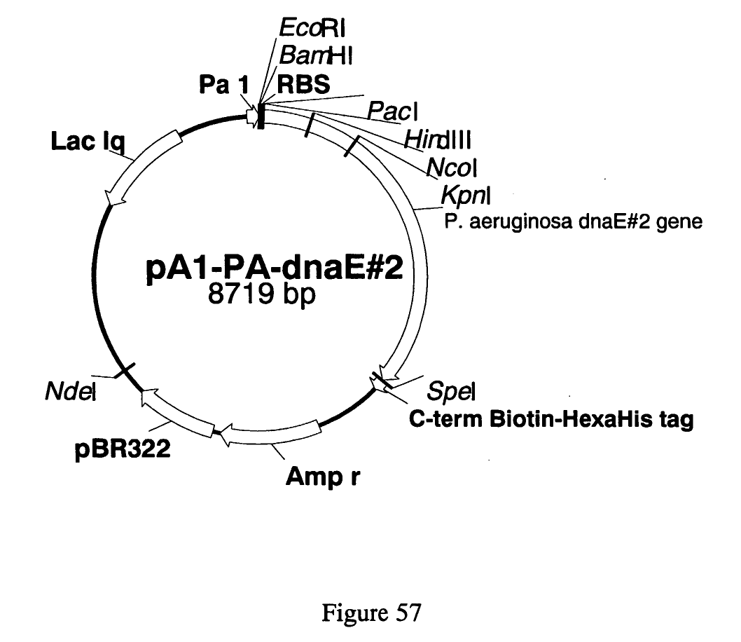

The PCR product is digested with KpnI/SpeI and inserted into pA1-PA-dnaE#2-5′ that has been digested with the same two restriction enzymes. This places the entire dnaE #2 gene in the plasmid with everything in the correct reading frame. This plasmid is named pA1-PA-dnaE#2 and is graphically shown in FIG. 57.

### 42.3. Expression of dnaE in E. coli

P. aeruginosa DnaE protein (a subunit) will be expressed in E. coli by cloning it into vectors similar to those used to overexpress E. coli DnaE (Kim, D. R. and McHenry, C. S. (1996) J Biol Chem 271, 20681-20689), Tth DnaE (Bullard, et al., ibid.) and S. pyogenes DnaE. Overexpression from 1-10% of the total cellular protein has been accomplished for these three a subunits.

Expression vecrors will contain synthetic promoters that are highly inducible and repressible by the lac repressor or pET-style vectors that are transcribed by T7 RNA polymerase (Kim, D. R. and McHenry, C. S. (1996) J Biol Chem 271, 20681-20689; Kim, D. R. and McHenry, C. S. (1996)J Biol Chem 271, 20690-20698; Marians, K. J. (1995) Methods Enzymol 262, 507-521). Portions of the P. aeruginosa dnaE gene will be obtained by PCR to place restriction sites at an appropriate location relative to the initiating ATG. All DNA obtained by PCR will be verified by DNA sequencing to ensure that no mutations were created by the amplification procedures. Toxicity after induction should not be a problem—100 mg quantities of over-expressed proteins are routinely purified from dead cells grown to high density before induction. However, if toxicity prior to induction with accompanying plasmid loss or mutation becomes a problem, an alternative approach may be used, such as T7 lysozyme co-expression or growing cells without any endogenous T7 RNA polymerase and introducing the enzyme by infection with recombinant M13 phage expressing T7 RNA polymerase prior to induction. Similar procedures have been performed up to the 180 l scale in a fermentor. No special difficulties in expressing this protein are expected since the highly homologous E. coli, T. thermophilus and S. pyogenes proteins have been expressed in E. coli and purified (Kim, D. R. and McHenry, C. S. (1996) J Biol Chem 271, 20681-20689). Cells will be grown in a standard fermentor, induced, lysed by standard protocols discussed above in Example 4 and the P. aeruginosa DnaE protein purified (Ibid.). The polymerase is expected to display properties similar to the homologous E. coli protein. The resulting purified enzyme will serve as the first expressed elongation protein essential for the reconstituted P. aeruginosa replication system. As the subunit that provides the DNA synthesis catalytic activity, DnaE will be an essential component for the reconstitution assay since modulation of its activity will be used to measure the activity of added auxiliary proteins.

Purification for P. aeruginosa alpha will be accomplished with reference to previous work. Enzyme recovery and location can be monitored using an art-known simple assay that does not require auxiliary factors—the nonprocessive filling of gaps in nuclease-activated DNA (Ibid.). Generally, DNA polymerase III a subunits are relatively insoluble in ammonium sulfate. The lowest concentration of ammonium sulfate that precipitates at least 90% of P. aeruginosa DnaE will be used. The majority of E. coli protein will be left in the supernatant. Cation exchange chromatography generally gives good purification of polymerases. They bind under conditions where most E. coli proteins flow through and are eluted with high purification in a salt gradient. Obtaining pure protein at this stage is anticipated, but if it is not achieved, additional art-known chromatographic steps will be developed that achieve maximal purification with preservation of activity. Total protein levels will be determined by the Bradford procedure (Bradford, M. M. (1976) Anal Biochem 72, 248-254) in this and other purifications described in this application.

### Example 43

### Molecular cloning of P. aeruginosa dnaQ, the Gene Encoding DnaQ (Epsilon) Subunit Protein

This example describes PCR amplification of the dnaQ gene, which encodes the epsilon (ε) subunit, from P. aeruginosa genomic DNA. The dnaQ gene is then inserted into the pA1-CB-NdeI plasmid (polyclonal region of pA1-CB-NdeI is shown in FIG. 10). The dnaQ gene does not have any 5′ low usage codon for expression in E. coli. The ε subunit is composed of 246 amino acids. The gene encoding ε is dnaQ, and is composed of 741 nucleotides including the stop codon. The nucleotide sequence of dnaQ is represented by SEQ ID NO:75. The amino acid sequence of P. aeruginosa ε is represented by SEQ ID NO:77. The forward/sense primer is: 5′-agtcCATATGCGTAGCGTCGTACTGGATA-3′ (29 mer) (SEQ ID NO:78). There is a 4-nucleotide 5′ clamp (lowercase agtc) allowing efficient digestion by the restriction enzyme. An NdeI restriction site (upper case, italic letters) follows the clamp region. The NdeI site overlaps the ATG start codon for the dnaQ gene. The next 19 nucleotides corresponding to nucleotides 4-22 of the 5′ end of the dnaQ gene are shown as upper case. All nucleotides corresponding to the 5′ end of the dnaQ gene are underlined. The reverse/antisense primer is: 5′-agtcGCTAGCAAGCTTattCCTCCTctaCTATTCGCCGACCGGCGCCTCCA-3′ (51 mer) (SEQ ID NO:79). There is a 4-nucleotide 5′ clamp (lower case-agtc) allowing for efficient digestion by the restriction enzyme. Following the clamp is an NheI restriction site (upper case, italic letters) allowing insertion into pA1-CB-NdeI. The NheI restriction site is followed by a HindIII restriction site (also, uppercase, italic letters), which is used later in operon construction. Next, is a 3-nucleotide spacer (lowercase, boldface letters) that provides optimal spacing for the gene(s) added downstream of dnaQ in operon construction. This is follow by the RBS (uppercase, boldface letters). Following the RBS is a second non-complementary stop codon (lowercase, boldface letters) that is adjacent to the native stop codon, giving two stop codons in tandem. The last 23 nucleotides (upper-case, underlined) are complementary to the 3′ end of the dnaQ gene.

P. aeruginosa dnaQ gene is PCR amplified using the two primers and digested with NdeI/NheI restriction enzymes.

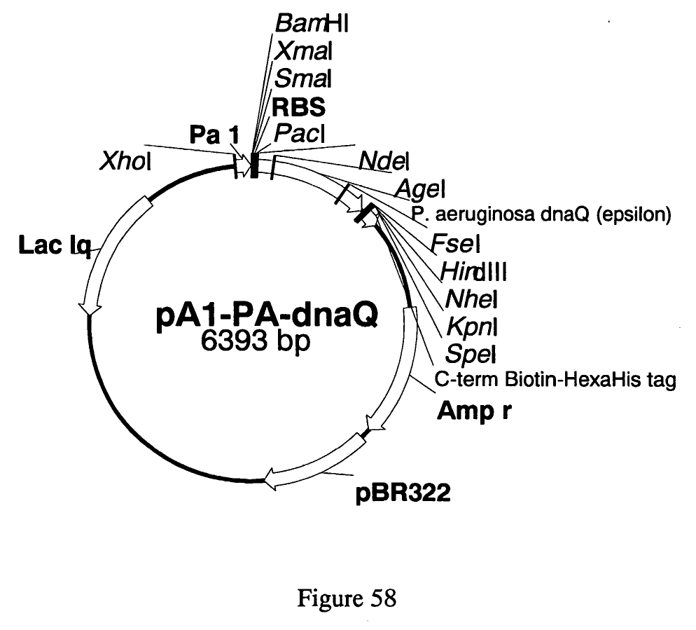

The digested PCR fragment is inserted into pA1-CB-NdeI that has been digested with the same two restriction enzymes (NdeI/NheI). This forms pA1-PA-dnaQ. The start codon is 11 nucleotides downstream of the RBS, which is optimal spacing. The pA1-PA-dnaQ plasmid is graphically shown in FIG. 58.

### Example 44

### Operon Cloning

### 44.1. Construction of 2-gene operon containing dnaE #1 and dnaQ

This example describes extracting the dnaE #1 gene out of pA1-PA-dnaE#1 and inserting it downstream of the dnaQ gene in the pA1-PA-dnaQ plasmid. In construction of pA1-PA-dnaQ a RBS and a HindIII site were placed downstream of the dnaQ gene allowing for operon construction. These components are utilized in construction of this operon.

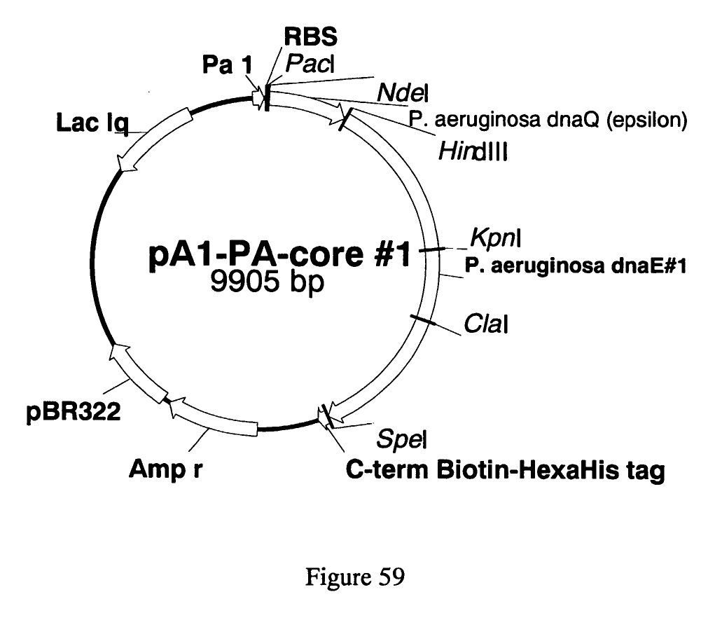

To extract the dnaE #1 gene from pA1-PA-dnaE#1, the plasmid is digested with HindIII/SpeI. The fragment containing the dnaE #1 gene (approximately 3.5 kb) is then inserted into pA1-PA-dnaQ that has been digested with the same two restriction enzymes. This places the dnaE #1 gene optimally spaced from the downstream RBS. This plasmid is named pA1-PA-core#1 and is graphically shown in FIG. 59.

### 44.2. Construction of 2-Gene Operon Containing dnaE#1 and dna Q

This example describes extracting the dnaE #2 gene out of pA1-PA-dnaE#2 and inserting it downstream of the dnaQ gene in the pA1-PA-dnaQ plasmid. In construction of pA1-PA-dnaQ, a RBS and a HindIII restriction site were placed downstream of the dnaQ gene allowing for operon construction. These components are utilized here. The steps here are exactly the same as in construction of pA1-PA-core#1 except with dnaE#2 is used instead of dnaE#1.

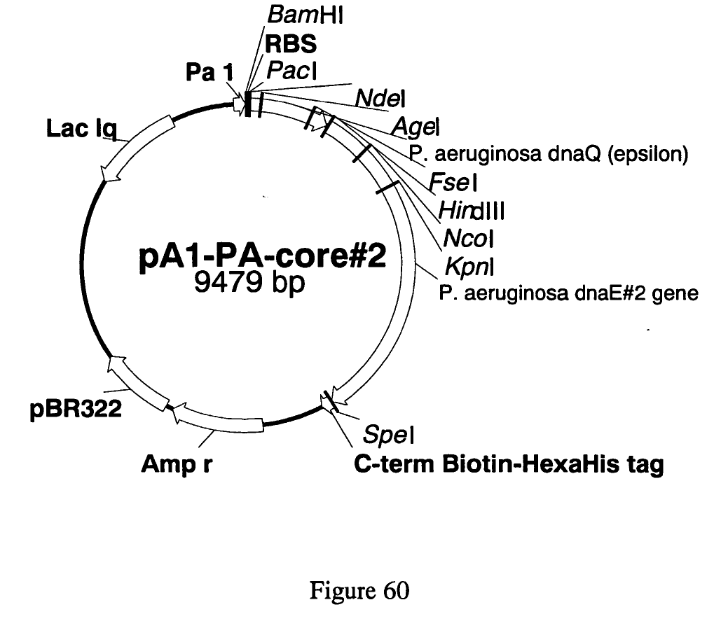

To extract the dnaE #2 gene from pA1-PA-dnaE#2, this plasmid is digested with HindIII/SpeI. The fragment containing the dnaE #2 gene (approximately 3 kb) is inserted into pA1-PA-dnaQ that has been digested with the same two restriction enzymes. This places the dnaE #2 gene optimally spaced from the downstream RBS. This plasmid is named pA1-PA-core#2 and is graphically shown in FIG. 60.

Beta Clamp

### Example 45

### Molecular Cloning of P. aeruginosa dnaN, the Gene Encoding the P. aeruginosa DnaN (β Subunit) Processivity Factor, its and Expression in E. coli, its Purification, and Characterization of its Contribution to the Processivity of DnaE

The P. aeruginosa dnaN gene encoding the β subunit of DNA pol III holoenzyme has been identified and annotated in the database (Stover, et al., ibid.). Comparison of amino acid sequences for DnaN proteins from P. aeruginosa and E. coli shows that they are 56% identical and align over the entire length of the protein sequence (FIG. 4).

This example describes amplification of the dnaN gene (β) from P. aeruginosa genomic DNA using PCR and then inserting it into the vector pA1-CB-NdeI. The region of pA1-CB-NdeI encompassing the polyclonal region is shown in FIG. 10. The β subunit is composed of 367 amino acids. The gene encoding β is dnaN, and is composed of 1104 nucleotides (nts) including the stop codon. All of the primers for all projects are quite long, up to 61 nucleotides, and require gel or HPLC purification. The nucleotide sequence of dnaN is represeneted by SEQ ID NO:112. The amino acid sequence for β is reperesented by SEQ ID NO:114. The forward/sense primer is:

| | | |
|---|---|---|
| 5′-agtcTTAATTAAcATGCATTTCA | (28mer) | (SEQ ID NO:115) |
| CCATTCAACGCGAA-3′. | | |

There is a 4-nucleotide 5′ clamp (lowercase agtc) allowing efficient digestion by the restriction enzyme. This is followed by a PacI restriction site for insertion into pA1-CB-NdeI (uppercase boldface letters) and a one nucleotide spacer “c”, shown as double underlined, for optimal spacing between the ribosome binding site (RBS) and the start codon. The next six nucleotides (capitalized italics) form an Nsil restriction site, which is not used. These six nucleotides are also the first six nucleotides of the dnaN gene sequence and are followed by 18 nucleotides (upper case) that correspond to nucleotides 7-24 of the 5′ end of the dnaN gene. All nucleotides corresponding to the dnaN gene are since underlined. There are no E. coli low usage codons in the 5′ end of the dnaN gene; therefore the primer is complementary to the terminal 5′ end of the gene. The reverse/antisense primer is:

| | | |
|---|---|---|
| 5′-agtcACTAGTttaTTAGAGGCGC | (36mer) | (SEQ ID NO:116) |
| ATCGGCATGACGA-3′. | | |

There is a 4-nucleotides 5′ clamp (lowercase agtc) allowing efficient digestion by the restriction enzyme. The clamp is followed by a SpeI restriction site, which is used for insertion into pA1-CB-NdeI (upper case italic letters). Next, there is a second non-complementary stop codon (lower case, bold, double underlined letters) in tandem with the native stop codon. This is followed by 23 nucleotides complementary to the 3′ end of the dnaN gene.

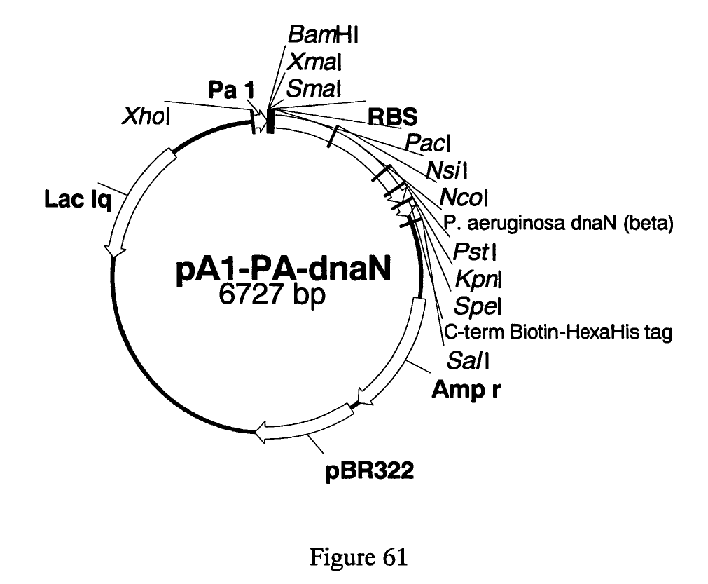

Both the P. aeruginosa dnaN PCR product and the plasmid pA1-CB-NdeI are digested with PacI/SpeI restriction enzymes. The fragment of the PCR product containing the dnaN gene (1.1 kb) is inserted into the PacI/SpeI digested pA1-CB-NdeI. This places the dnaN gene optimally spaced from the upstream RBS and results in the plasmid pA1-PA-dnaN, which is graphically shown in Figure FIG. 61.

Purifications will be modeled after the successful procedures developed for the E. coli (Johanson, K. O., Haynes, T. E., and McHenry, C. S. (1986) J Biol Chem 261, 11460-11465) and T. thermophilus β (Bullard, et al., ibid.) and adapted, as necessary, for the unique characteristics of P. aeruginosa β. The purification procedure will be developed empirically, using the functional processivity assay on linear templates to locate and quantify P. aeruginosa β. Purified β, together with a will provide the basis for developing assays for purification of the essential components of the P. aeruginosa DnaX complex.

Clamp Loading Complex

### Example 46

### Molecular Cloning of P. aeruginosa DnaX, HolA and HolB Components of the P. aeruginosa DnaX Complex, Their Expression in E. coli, and Their Purification

The gene for P. aeruginosa dnaX gene of DNA pol III holoenzyme has been identified and annotated in the database (Stover, et al, ibid.). Comparison of amino acid sequences for DnaX proteins from P. aeruginosa and E. coli shows that they are 45% identical and align over the entire length of the protein sequence, with the highest sequence identity occurring in the amino-terminal region of the protein (FIG. 5).

This example describes PCR amplifying the dnaX gene, which encodes the τ subunit, from P. aeruginosa genomic DNA. The dnaX gene is then inserted into the pA1-CB-NcoI plasmid (polyclonal region of pA1-CB-NcoI is shown in FIG. 31). The dnaX gene does not have a translational frame-shifting site, as does the dnaX gene from E. coli and Y. pestis, therefore internal mutations to change any coding region that might contain a frame-shifting site have not been performed. The τ subunit is composed of 681 amino acids. The gene encoding DnaX (τ) is dnaX, and is composed of 2046 nucleotides including the stop codon. The nucleotide sequence of dnaX is represented by SEQ ID NO:80. Codon #2, which codes for Ser, is a low usage codon in E. coli and is therefore changed by the forward/sense primer to a high usage codon. The #2 codon is changed from AGT>AGC. The amino acid sequence of DnaX (τ) is represented by SEQ ID NO:82. The forward/sense primer is: 5′-agtaTTAATTAAGCTTATGAGCTATCAAGTTCTTGCGCGTAAAT-3′ (44 mer) (SEQ ID NO:83). There is a 4-nucleotide 5′ clamp (lower case agta) allowing for efficient digestion by the restriction enzyme. The clamp is followed by a PacI site (upper case, italic letters), which overlaps the first two nucleotides of a HindIII restriction site (also shown as upper case, italic letters). Next, is the start codon and the codon #2 that is modified to a high usage codon (AGT>AGC), both are shown as upper case, bold print. The final 22 nucleotides that correspond to positions 7-28 of the dnaX gene are shown as upper case and underlined print. The reverse/antisense primer is: 5′-agtcACTAGTctaTCAGGCCTTGGCTTCCAAA-3′ (32 mer) (SEQ ID NO:84). There is a 4-nucleotide 5′ clamp (lower casagtc) allowing for efficient digestion by the restriction enzyme. Following the clamp is a SpeI restriction site (uppercase, italic letters). Next, is a second non-complementary stop codon (lowercase, boldface letters) that is adjacent to the native stop codon and creates tandem stop codons. The next 19 nucleotides (upper case, underlined) are complementary to the last 19 nucleotides of the dnaX gene.

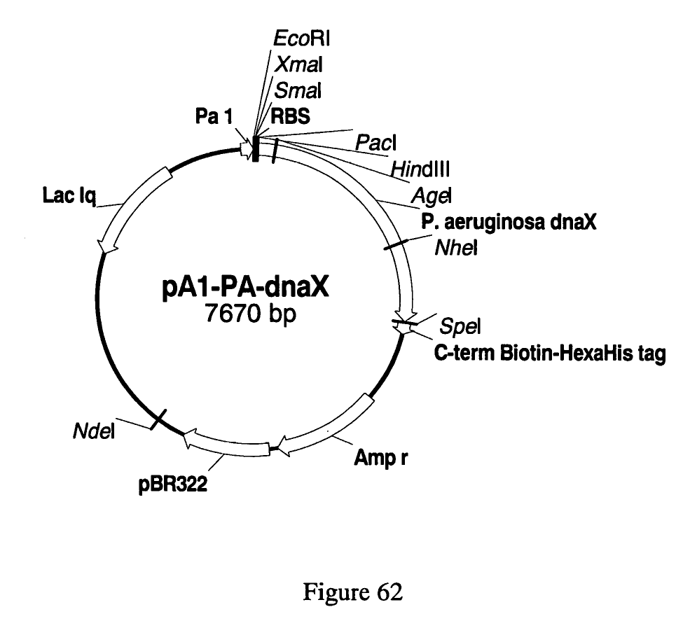

P. aeruginosa dnaX gene is PCR amplified using the two primers and digested with PacI/SpeI restriction enzymes. The digested PCR fragment is then inserted into pA1-CB-NcoI that has been digested with the same two restriction enzymes. This forms pA1-PA-dnaX. The start codon will be 12 nucleotides downstream of the RBS, which is only 1 nucleotide above optimal spacing and will express well in its own right. However, once inserted in the operon downstream of holB (δ′) and holA (δ), the spacing becomes optimal. pA1-PA-dnaX is graphically shown in FIG. 62.

### Example 47

### Molecular Cloning of P. aeruginosa holA, the Gene Encoding HolA (Delta) Subunit

The genes for P. aeruginosa holA gene of DNA pol III holoenzyme has been identified and annotated in the database (Stover, et al., ibid.). Comparison of amino acid sequences for δ subunits from P. aeruginosa and E. coli shows that they are 34% identical and align over the entire length of the protein sequence (FIG. 6). P. aeruginosa δ will be cloned by PCR, expressed and purified, using methods similar to those described for DnaE.

This example describes amplifying the holA gene from P. aeruginosa genomic DNA using PCR. The holA gene encodes the δ subunit. The holA gene is then inserted into the pA1-CB-NcoI plasmid (polyclonal region of pA1-CB-NcoI is shown in figure P2). In the PCR amplification using the reverse/antisense primer sequences are added downstream of the stop codon that corresponds to the RBS site, and also restriction sites that can be used to add other genes downstream of the holA gene in operon formation. The δ subunit is composed of 345 amino acids. The gene encoding δ is holA, and is composed of 1038 nucleotides including the stop codon. The nucleotide sequence of holA is represented by SEQ ID NO:85. Codons #2, 4 and 5 (that code for Lys, Thr and Pro, respectively) are shown as double underlined, they are low usage codons in E. coli and are changed to high usage codons by the forward/sense primer. The change will be codon #2 AAG>AAA, codon #4 ACT>ACC and codon #5 CCC>CCG. The amino acid sequence for δ is represented by SEQ ID NO:87. The forward/sense primer is: 5′-agtaTTAATTAAGCTAGCatgaaactgaccccgGCGCAACTCGCCAAGCACCT-3′ (53 mer) (SEQ ID NO:88). There is a 4-nucleotide 5′ clamp (lower case-agta) to allow efficient digestion by the restriction enzyme. The restriction site for PacI (uppercase italic letters) follows the clamp and is used for insertion into pA1-CB-NcoI. This is followed be a restriction site for NheI (uppercase bold, italic letters). This restriction site is used in operon construction. Codons #2, 4 and 5 are low usage codons in E. coli and are changed by the forward/sense primer to high usage codons. These codons do not totally correspond to the holA gene and they are shown as lower case and bold. Finally, the last 20 nucleotides (upper case, underlined) correspond to nucleotides 16-35 of the holA gene. The reverse/antisense primer is: 5′-agtcACTAGTattAAGCTTtatCCTCCTctaTCAGGCTGCCGGCAGGCCGA-3′ (SEQ ID NO:89). There is a 4-nucleotide 5′ clamp (lower case-agtc) allowing for efficient digestion by the restriction enzyme. The clamp is followed by a SpeI site (uppercase, italic letters), which allows insertion into pA1-CB-NcoI. The SpeI restriction is followed by a three-nucleotide spacer (lower case, bold), which serves as a clamp region for restriction enzyme digestion of both the SpeI and the HindIII during operon construction. The HindIII restriction site (uppercase, italic letters) is next. The HindIII restriction site is followed by a three-nucleotide spacer (lowercase, bold), which provides optimal spacing between the RBS (upper case, bold) and a downstream gene during operon construction. Following the RBS is a second non-complementary stop codon, which is adjacent to the native stop codon, giving two stop codons in tandem. The final 20 nucleotides are complementary to the 3′ end of the holA gene and are shown as upper case and underlined.

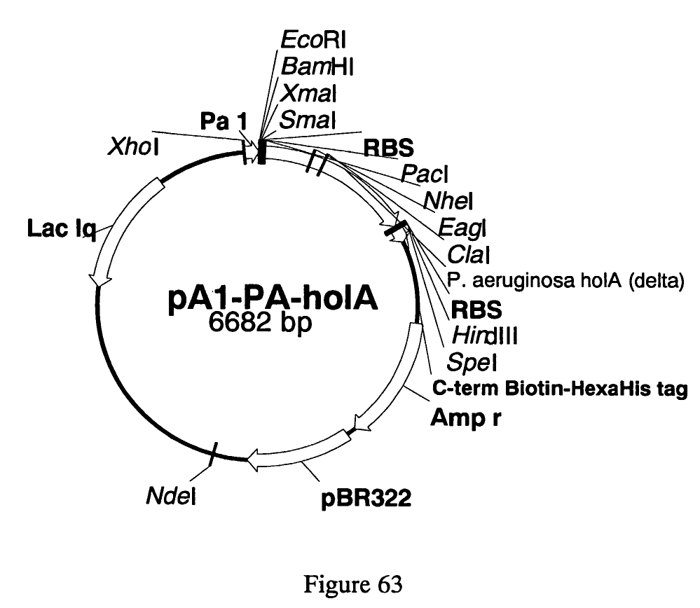

P. aeruginosa holA gene is amplified by PCR using the two primers and digested with PacI/SpeI restriction enzymes. The digested PCR fragment is inserted into pA1-CB-NcoI, which has also been digested with the same two restriction enzymes. This forms pA1-PA-holA. The start codon is 14 nucleotides downstream of the RBS, which is above optimal spacing. However, once inserted in the operon, the spacing becomes optimal. Also, the reverse/antisense primer has added a downstream RBS and restriction sites that are used in operon construction. The newly constructed pA1-PA-holA plasmid is graphically shown in FIG. 63.

### Example 48

### Molecular Cloning of P. aeruginosa holB, the Gene Encoding HolB (Delta Prime) Subunit

The gene for P. aeruginosa holB gene of DNA pol III holoenzyme has been identified and annotated in the database (Stover, et al., ibid.). Comparison of amino acid sequences for δ′ subunits from P. aeruginosa and E. coli shows that they are 31% identical and align over the entire length of the protein sequence (FIG. 6).

P. aeruginosa δ′ subunit will be cloned by PCR, expressed and purified, using methods similar to those described for DnaE.

In this project the holB gene, which encodes the δ′ subunit, is amplified from P. aeruginosa genomic DNA. The holB gene is inserted into the pA1-CB-NcoI plasmid. In the PCR product sequences are added by the reverse/antisense primer downstream of the stop codon that correspond to an RBS site, and also restriction sites that are used to add other genes downstream of the holB gene in operon formation. The region of pA1-CB-NcoI encompassing the polyclonal region is shown in FIG. P2. The δ′ subunit is composed of 328 amino acids. The gene encoding δ′ is holB, and is composed of 987 nucleotides, including the stop codon. The nucleotide sequence of holB is represented by SEQ ID NO:90. The amino acid sequence for ρ is represented by SEQ ID NO:92. The forward/sense primer is: 5′-agtcCCATGGCTGATATCTATCCCT-3′ (25 mer) (SEQ ID NO:93). There is a 4-nucleotide 5′ clamp (lower case-agtc) to allow efficient digestion by the restriction enzyme. The restriction site for NcoI (upper case italics) follows the clamp. The NcoI restriction site overlaps the first four (4) nucleotides of the 5′ end of the holB gene. This gives optimal spacing of the start codon from the RBS. The nucleotides corresponding to the 5′ end of the holB gene are underlined. There are no E. coli low usage codons in the 5′ end of the holB gene; therefore a primer that is complementary to the terminal 5′ end of the gene was used. The reverse/antisense primer is: 5′-agttACTAGTtatGCTAGCtttCCTCCTctaCTAGCCCGGCCCGGGCAGGCTT-3′ (53 mer) (SEQ ID NO:94). There is a 4-nucleotide 5′ clamp (lowercase agtt), followed by a SpeI restriction site (capitalized italic letters) for insertion into pA1-CB-NcoI. Next, there is a three-nucleotide spacer (double underline letters) for efficient digestion by restriction enzymes in operon construction. Following the spacer is an NheI restriction site (capitalized italic letters) used for operon construction. This is followed by another three-nucleotide spacer (lowercase, double underlined letters) that provides optimal spacing between the following RBS (capitalized, boldface letters) and the start codon of the downstream gene in operon construction. Following the RBS is a second stop codon that is non-complementary (lower case) and is in tandem with the native stop codon. This is followed by 22 nucleotides complementary to the 3′ end of the holb gene. The region of the primer complementary to the 3′ end of the holB gene is single underlined.

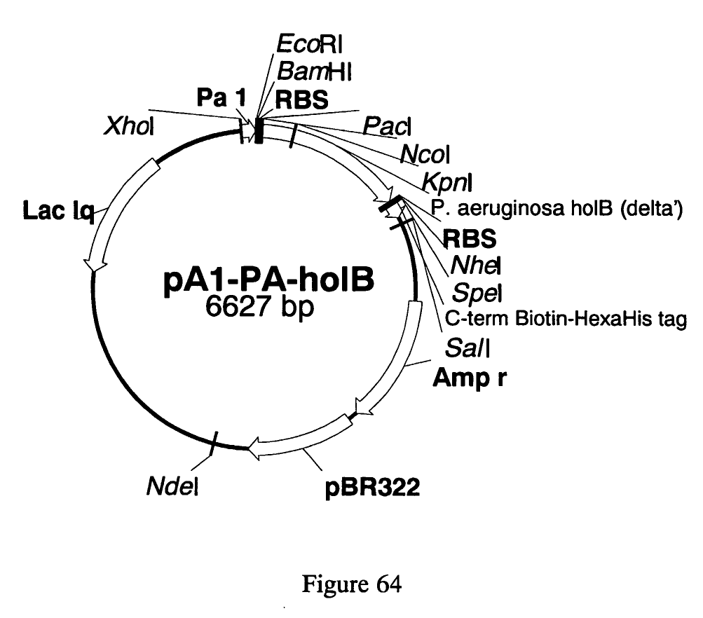

Both the P. aeruginosa holB PCR product and the plasmid pA1-CB-NcoI are digested with NcoI/SpeI restriction enzymes. The fragment of the PCR product containing the holB gene is inserted into the NcoI/SpeI digested pA1-CB-NcoI. This places the holb gene optimally spaced from the upstream RBS. Also, a RBS and sequences to allow a gene to be inserted in operon construction was placed downstream of the holB gene by the reverse/antisense primer. This plasmid is named pA1-PA-hoB, which is graphically shown in FIG. 64.

### Example 49

### Molecular Cloning of P. aeruginosa holC, the Gene Encoding HolC (Chi) Subunit

holC (χ) is expressed as a tagged protein. χ is expressed with both an N-terminal and a C-terminal tag. Also, the gene holC, which encodes χ, is inserted in two different vectors. First, holC is placed in the normal expression vectors containing an N-terminal or C-terminal tag and engineered so that it can be extracted from these vectors and placed in a broad host range vector.

### 49.1. Construction of Vector Containing N-terminal Tag

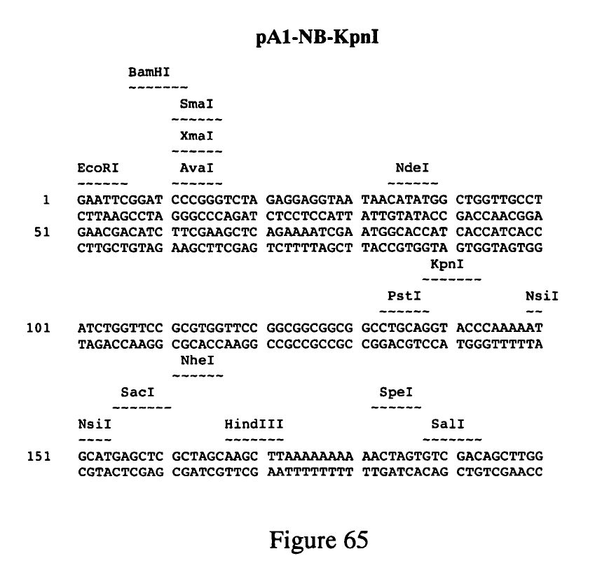

In this first method the vector was designed so that χ was expressed as an N-terminal tagged protein. Here PCR amplifying the holC gene, which encodes the χ subunit, from P. aeruginosa genomic DNA, is described. The holC gene was then inserted into the pA1-NB-KpnI plasmid (polyclonal region of pA1-NB-KpnI is shown in FIG. 65). The χ subunit is composed of 142 amino acids. The gene encoding χ is holC, and is composed of 429 nucleotides including the stop codon. The nucleotide sequence of holC is represented by SEQ ID NO:95. Codons #land 3 (that code Met and Arg, respectively) are shown as double underlined, they are low usage codons in E. coli and are changed to high usage codons by the forward/sense primer. The change is codon #1 GTG>ATG, codon #2 CGG>CGC. In the first vector holC is cloned as an N-terminal tagged protein, which begins at codon#2, so the start codon is omitted. The amino acid sequence for χ is SEQ ID NO:97. The forward/sense primer is: 5′-gatcATGCATacccgcGTCGATTTCTACGTGATCCCCA-3′ (38 mer) (SEQ ID NO:98). There is a 4-nucleotide 5′ clamp (lower case-gatc) allowing efficient digestion by the restriction enzyme. Following the clamp is an NsiI restriction site (upper-case, italic letterss). This NsiI restriction site will be used to insert the gene into pA1-NB-KpnI. Next, there is codon #2 and #3, which do not correspond to codon #2 and #3 of holC because codon #3 is modified from CGG>CGC. The final 22 nucleotides correspond to positions 10-31 of holC and are shown as upper case underlined. The reverse/antisense primer is: 5′-gcatACTAGTGAGCTCtcaTCAGATACGCGGCAGGCGAT-3′ (SEQ ID NO:99). There is a 4-nucleotide 5′ clamp (lowercase gcat) allowing efficient digestion by the restriction enzyme. Following the clamp is a SpeI restrictions site (uppercase, italic letters) for insertion into pA1-NB-KpnI. Adjacent to the SpeI site is a SacI restriction site (also uppercase, italic letters), which is used in the cut and paste (digestion and ligation) reaction to insert N-terminal tagged holC into the broad host range vector. Next is a second non-complementary stop codon (lowercase, boldface letters) that will be adjacent to the native stop codon. The final 20 nucleotides (uppercase, underlined letters) are complementary to the 3′ end of the holC gene. P. aeruginosa holC gene was PCR amplified using the two primers.

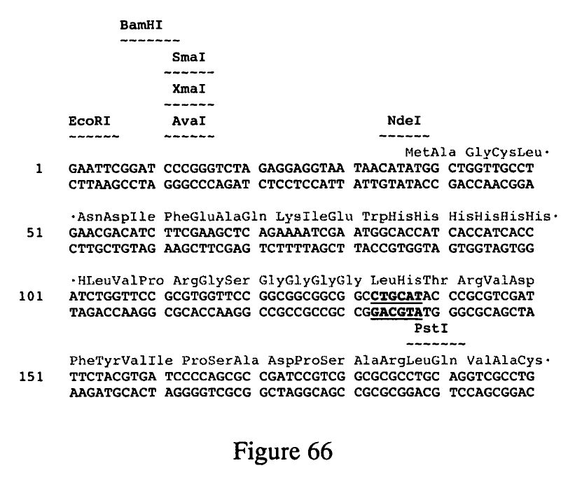

The PCR product was digested with NsiI/SpeI and the fragment (approximately 0.5 kb) containing the holC gene was inserted into pA1-NB-KpnI. There is an NsiI site in the polyclonal region of pA1-NB-KpnI, but this site was not used for insertion of holC. Instead pA1-NB-KpnI was digested with PstI/SpeI. The digested PstI restriction site of the plasmid and the NsiI restriction site of the PCR product were annealed together and were ligated even though both sites were destroyed by the reaction. This placed codon #2 in the same reading frame with the N-terminal tag and allowed holC to be expressed as an N-terminal tagged protein. The reason that PstI site in the PCR reaction was not used is that there is an internal PstI within the holC gene. This ligated region is shown in FIG. 66.

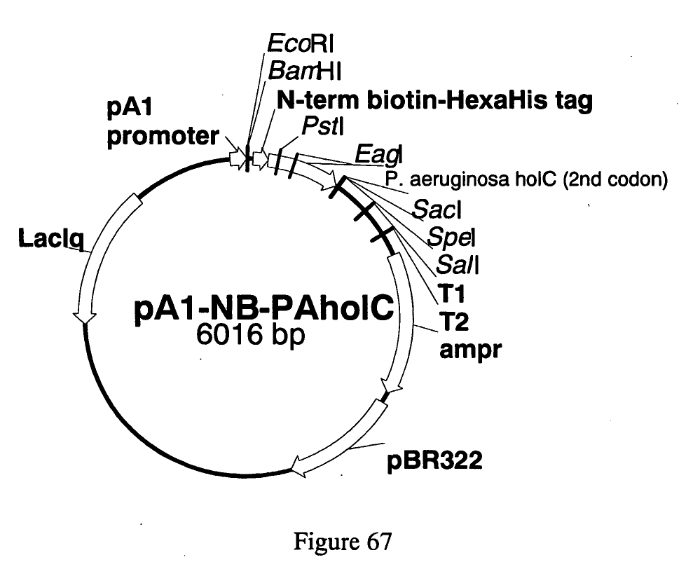

The region where PstI and NsiI were meshed together is shown as bold and underlined. The first 5 nucleotides of the CTGCAT of the sequence of PstI restriction site and the last 5 nucleotides of the ATGCAT of the NsiI restriction site were meshed to form CTGCAT. This destroyed both the PstI and the NsiI restriction sites. Also, the internal PstI restriction site is shown in the bottom line of sequence. This plasmid was named pA1-NB-PAholC and a graphic display is shown in FIG. 67.

The plasmid was transformed into DH5α bacteria and plasmids from ampicillin-resistant positive isolates were screened for by digestion with NdeI and SacI restriction enzymes yielding 0.6 and 5.4 kb fragments. The sequence of both strands of the insert were verified by DNA sequencing. Sequence analysis confirmed that the correct sequence was contained within the inserted region.

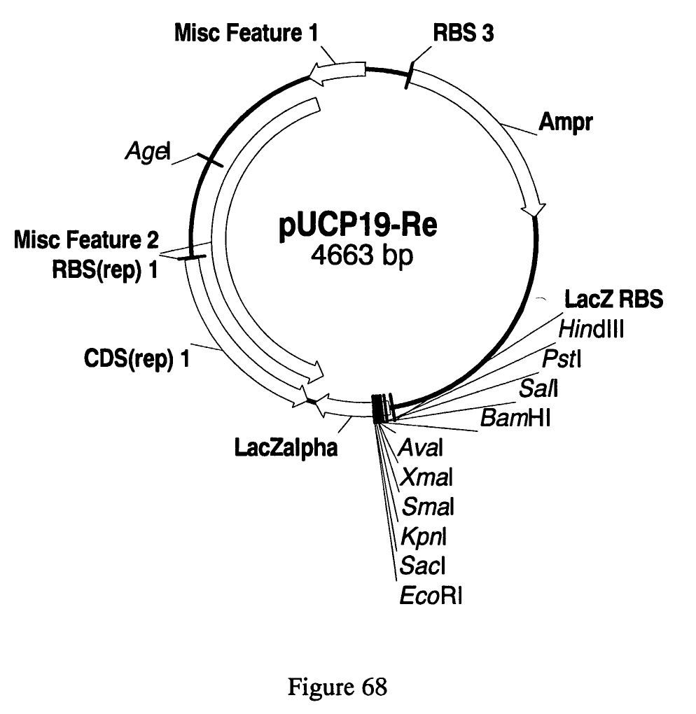

The entire region containing the RBS, N-terminal tag and the holC gene were extracted from pA1-NB-PAholC. This was accomplished by digesting pA1-NB-PAholC with BamHI/SacI. From the ligated region shown in FIG. 65, the BamHI site can be seen upstream of the N-terminal region and SacI was added just downstream of the holC stop codon in the PCR reaction. The fragment containing the RBS, N-terminal tag and holC (approximately 0.6 kb) was inserted into the broad host range plasmid pUCP19 digested with BamHI/SacI. This vector is graphically shown in FIG. 68.

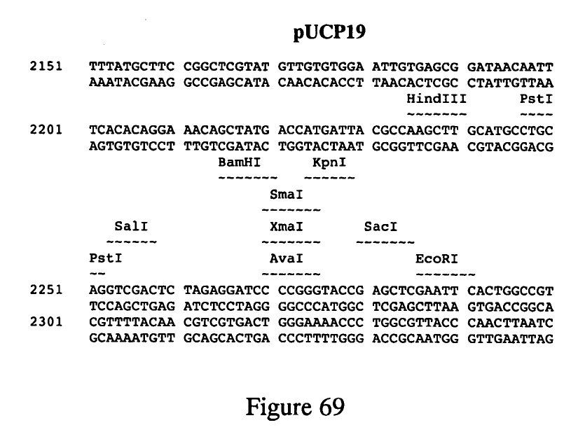

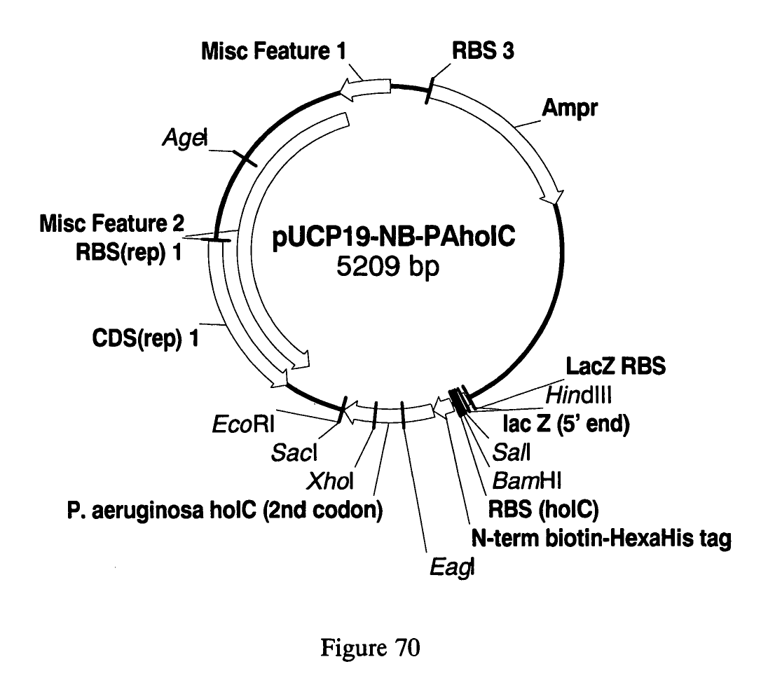

The polyclonal region for pUCP19 is the same as the polyclonal region from the pUC19 plasmid, and is within the LacZ gene in pUCP19. This plasmid contains both a pBR322 origin of replication and an origin of replication form P. aeruginosa; this allows replication in both E. coli and P. aeruginosa. The initial plasmid was pUC19 and a 1.9 kb fragment from the region surrounding the P. aeruginosa origination of replication (which included portions of two P. aeruginosa genes) was inserted into pUC19 to form pUCP19 (West, S. E. H., et. al., (1994), Gene, 128, 81-86). The polyclonal region is shown in FIG. 69. The pUCP19 plasmid was digested with the same two restriction enzymes (BamHI and SacI) used to digest pA1-NB-PAholC. The BamHI/SacI fragment (0.6 kb) from pA1-NB-PAholC was inserted into the digested pUCP-19. This placed the RBS, N-terminal tag and holC within the LacZ gene and under control of the LacZ promoter. The holC gene in this plasmid is needed to express at very low levels, since the native holD gene (ψ) will be expressed at very low levels in P. aeruginosa cells. If expression is too great, it will be difficult to resolve enough ψ from the χ expressed to do amino acid sequencing. A graphical representation of this new plasmid named pUCP-NB-PAholC is shown in FIG. 70.

The plasmid was transformed into DH5α bacteria and plasmids from ampicillin-resistant positive isolates were screened for by digestion with BamHI and SacIrestriction enzymes yielding 0.6 and 4.5 kb fragments. The sequence of both strands of the insert were verified by DNA sequencing. Sequence analysis confirmed that the correct sequence was contained within the inserted region.

### 49.2. Construction of Vector Containing C-Terminal Tag

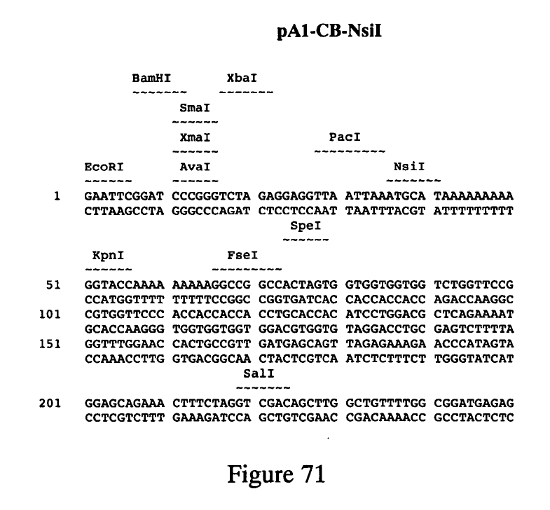

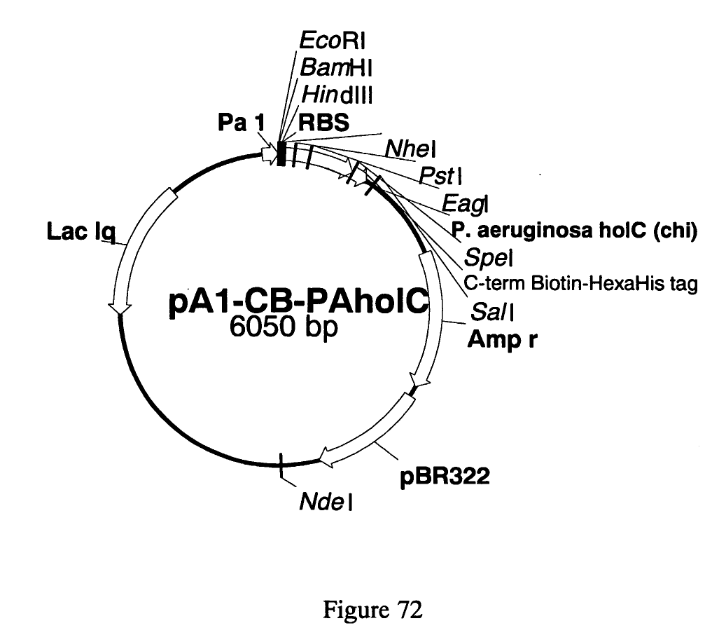

This example describes the design of a vector so that P. aeruginosa χ can be expressed as a C-terminally tagged protein. The holC gene, which encodes the χ subunit, is PCR amplified from from P. aeruginosa genomic DNA. The holC gene is then inserted into the pA1-CB-NsiI plasmid (polyclonal region of pA1-NB-NsiI is shown in FIG. 71). To PCR amplify the holC gene for insertion into pA1-CB-NsiI, the forward/sense primer is: 5′-gtcaTCTAGAAGCTTAGGAGGaccGCTAGCATGACCCGCGTCGATTTCTACGTGATCC CCA-3′ (61 mer) (SEQ ID NO:100). There is a 4-nucleotide 5′ clamp (lowercase gtca) allowing efficient digestion by the restriction enzyme. The clamp is followed by an XbaI restriction site (uppercase italic letters), which is used for insertion into pA1-CB-NsiI. Following the XbaI restriction site and overlapping the last nucleotide is a HindIII restriction site (also uppercase, italic letters), which is used to insert the gene encoding the C-terminal tagged holC into the pUCP19 plasmid. Next, there is a RBS (uppercase, boldface letters), which replaces the one removed by insertion of holC into pA1-CB-NsiI. A three-nucleotide spacer and then an NheI restriction site follow the RBS. Both the spacer and the NheI restriction site serve as a spacer giving optimal spacing between the RBS and the start codon for holC. Next, are the first three codons of the 5′ end of the holC gene (upper-case, bold). They are not underlined because codons #1 and #3 have been modified for high usage in E. coli (codon #1 changed from GTG>ATG and codon #3 from CGG>CGC, respectively encoding Met and Arg). The final 22 nucleotides correspond to positions 10-31 of holC and are shown as upper case and underlined. The reverse/antisense primer is: 5′-gactACTAGTGATACGCGGCAGGCGATGGT-3′ (30 mer) (SEQ ID NO:101). There is a 4-nucleotide 5′ clamp (lowercase gact) to allow efficient digestion by the restriction enzyme. A SpeI restriction site for insertion into pA1-CB-NsiI follows the clamp. The stop codon has been omitted and the penultimate codon will be adjacent to the SpeI restriction site, which will maintain an open-reading frame from the holC gene into the C-terminal tag sequence. This will allow the χ protein to be expressed as a C-terminally tagged protein. The final 20 nucleotides are complementary to the 3′ end of the holC gene up to but not including the stop codon. The PCR product is digested with XbaI/SpeI (approximately 0.5 kb) and inserted into pA1-CB-NsiI that has been digested with the same two restriction enzymes. When pA1-CB-NsiI is digested with XbaI/SpeI the region between the XbaI and the SpeI restriction sites, which contain the RBS and the polyclonal region, is removed. The forward/sense primer replaces the previously removed RBS with a new RBS that is properly spaced from the holC start codon. The C-terminal tag sequence is located between the SpeI and the SalI restriction site. A gene inserted in the same reading frame as the SpeI restriction site will be in the same reading frame as the C-terminal tag. Insertion of the digested PCR product into pA1-CB-NsiI results in the formation of pA1-CB-PAholC, which is graphically shown in FIG. 72. The correct sequence as described has been confirmed by DNA sequencing.

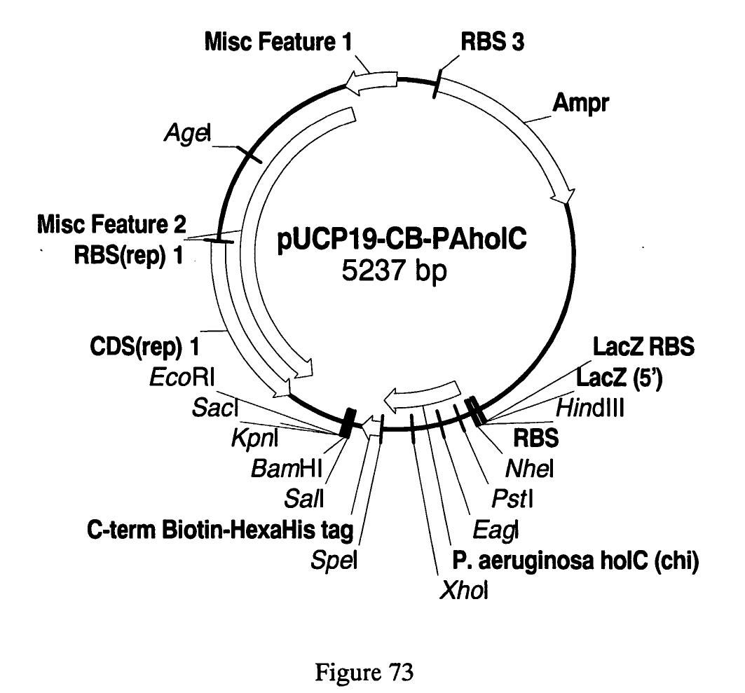

In the next step, the entire region containing the RBS, holC gene and the C-terminal tag sequence is extracted from pA1-CB-PAholC. This is accomplished by digesting pA1-CB-PAholC with HindIII/SalI. From the polyclonal region of pUCP19 shown in FIG. 69, the HindIII site can be seen upstream SalI restriction site. The fragment containing the RBS, holC (approximately 0.6 kb) and C-terminal tag sequence will be inserted into the HindIII/SalI restriction sites of the broad host range plasmid pUCP19. In this plasmid the P. aeruginosa holC gene is within the lacZ gene, but in a different reading frame. This results in the plasmid pUCP-CB-PAholC, which is graphically shown in FIG. 73. Since the holC gene will have its own RBS, a certain percentage of this protein is expressed, and if the LacZ gene does read through the start of the holC gene, it will terminate a few codons downstream. This holds true for both the pUCP19-CB-PAholC and pUCP19-NB-PAholC. Once the identity of holD (ψ) is known, a holC/holD operon can be prepared and then joined with the pA1-PA-BAX operon to form a clamp-loader operon.

Operon Cloning

### Example 50

### Construction Two Gene Operon Containing Both holB and holA

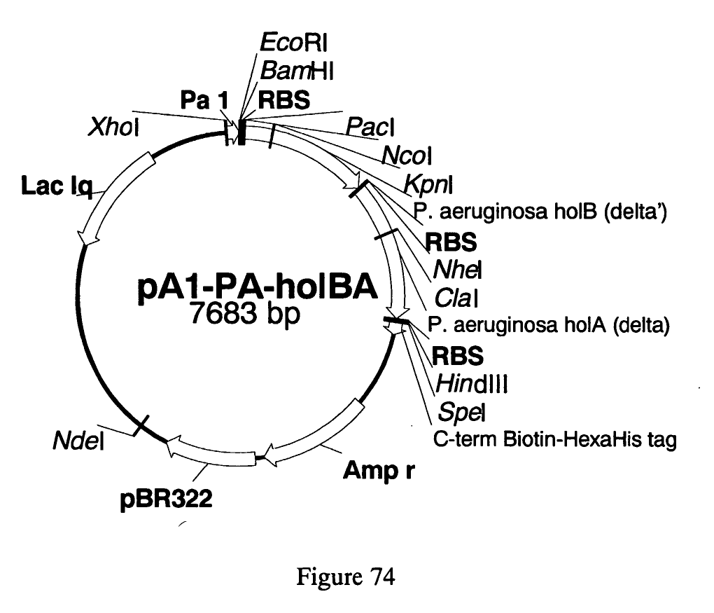

A preliminary operon containing both the genes for δ′ (holB) and δ (holA) is constructed by inserting the holA gene in pA1-PA-holA behind (downstream) of the holB gene in pA1-PA-holB. This is accomplished by digesting both pA1-PA-holA and pA1-PA-holB with NheI/SpeI. The fragment from pA1-PA-holA containing holA (approximately 1 kb) is then inserted into the digested pA1-PA-holB. This locates the holA gene downstream of holB and optimally placed downstream of an RBS that was previously placed downstream of holB. This also places the RBS created by the reverse/antisense primer in the construction of pA1-PA-holA downstream of the holA gene and in position for addition of another downstream gene in operon construction. This two-gene operon is named pA1-PA-holBA and is graphically shown in FIG. 74.

### Example 51

### Construction Three Gene Operon Containing holB, holA, and dnaX

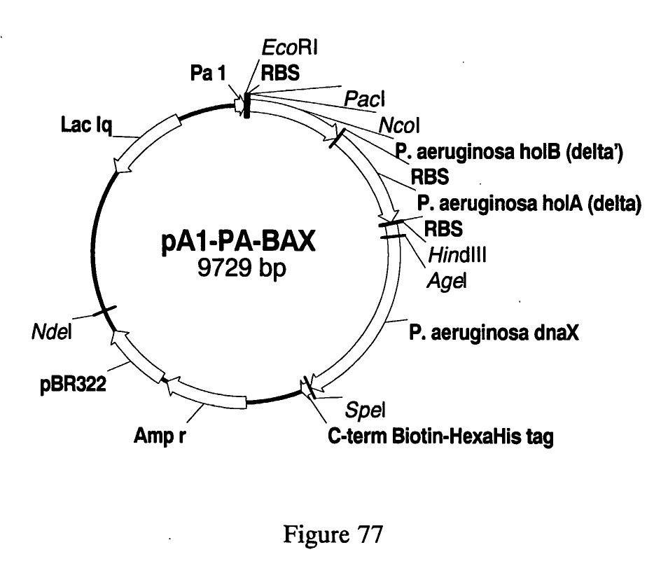

To construct the operon containing genes encoding δ′, δ and DnaX (τ), pA1-PA-dnaX and pA1-PA-holBA are both digested with HindIIISpeI restriction enzymes. The fragment of pA1-PA-dnaX containing the dnaX gene (approximately 2 kb) is inserted into the digested pA1-PA-holBA plasmid to generate pA1-PA-BAX, shown in FIG. 77. This places the P. aeruginosa dnaX gene downstream of the holA gene and also optimally spaced downstream of the RBS that was placed downstream of the holA gene in construction of pA1-PA-holA. This allows δ′, δ and DnaX (τ) to all be expressed as a three gene operon. This three-gene operon is eventually placed downstream of the holC/holD operon. However, all three proteins in this operon will express as they are all optimally spaced from their respective RBS.

### Example 52

### Purification of P. aeruginosa DnaX (τ), HolA (δ) and HolB (δ′) Proteins

The P. aeruginosa DnaX (τ), HolA (δ) and HolB (δ′) proteins will be purified using a reconstitution assay to monitor their purification. Although complex, these procedures have become more commonplace and are used in E. coli, T. thermophilus and S. pyogenes DNA replication studies. Routinely, expression at adequate levels can be obtained such that an extra coomassie-staining band can be observed upon SDS-PAGE. If this is not realized in these studies, expression might be verified with monoclonal antibodies directed against E. coli subunits. Significant cross-reactivity has been observed with selected antibodies against the holoenzyme subunits of more evolutionarily distant organisms in the past. Once overproducers of the DnaX, δ and δ′ subunits are in hand, cells will be lysed, ammonium sulfate pellets will be generated using optimal amounts of ammonium sulfate to guarantee precipitation of the sought subunits. The redissolved, dialyzed, pellets will be used for reconstituting P. aeruginosa DNA polymerase III holoenzyme to provide the basis for a quantitative functional assay to direct the purification of individual subunits.

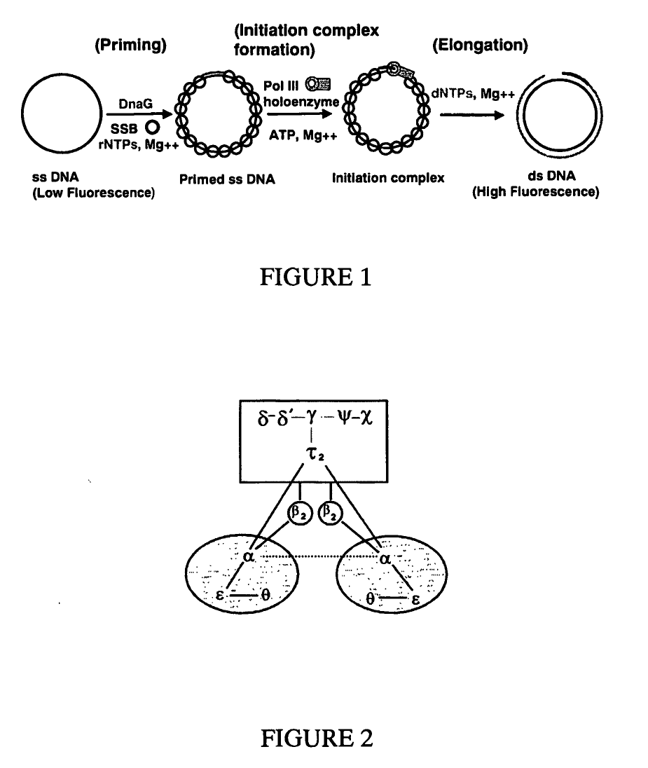

For an assay, a short DNA primer will be annealed to a single-stranded M13 template, and dNTPs, ATP, Mg⁺⁺, P. aeruginosa α (DnaE) and β (DnaN) and varying levels of ammonium sulfate cuts from P. aeruginosa DnaX, HolA and HolB overproducing cells will be added. This assay is essentially the same as described in FIG. 1, except a DNA primer is provided, bypassing the need for the initial DnaG primase-catalyzed priming step. Once reconstitution is achieved, the saturating level of each protein or extract will be determined. Once this is accomplished, an assay will be in hand for P. aeruginosa HolA, HolB and DnaX.

The minimum ammonium sulfate concentration that precipitates each activity will then be determined individually. Purification of DnaX will then proceed, using crude HolA and HolB in the assay. With all DnaX proteins examined to date, cation exchange chromatography yields highly purified protein. Specific conditions that yield highly purified DnaX will be determined. Additional chromatographic steps will be developed, if needed, to yield nearly homogeneous protein. Once highly purified DnaX is in hand, it will be added, along with purified DnaE and DnaN and crude HolA to provide an assay to direct the purification of HolB.

Hol B will be purified using purifications for E. coli HolB (δ′) and T. thermophilus HolB (Bullard, et al. ibid.) as a model. Once this cycle is complete, purified DnaE, DnaN, DnaX and HolB will be used to provide an assay for HolA. HolA will be purified using the logic described for the other proteins, until highly purified.

Backup approaches if additional factors are required. This approach has been successfully applied for diverse bacteria (E. coli, T. thermophilus and S. pyogenes) as well as bacteriophage T4 and human and yeast. Thus, it is highly likely that the approach will be successful. However, if a combination of the three expressed proteins is unsuccessful, extracts can be prepared from P. aeruginosa (preferably non-pathogenic strains), varied ammonium sulfate cuts can be added to the assay to obtain activity and the resulting stimulatory activities purified. When the first bacterial replication system was established, this approach worked in resolving all of the bacterial replication factors and purifying each of them by a resolution and reconstitution approach before overexpression of any of the protein components (Schekman, R., Weiner, A., and Kornberg, A. (1974) Science 186, 987-993). Once any novel factors are obtained with their purifications developed using their intrinsic activity to monitor them, they can be readily identified by mass spectrometry or sequencing by comparison to the proteins encoded by the sequenced P. aeruginosa. The identified genes will be expressed and the corresponding protein purified using the approach developed from P. aeruginosa extracts. This approach could yield one of the sought proteins if, for some reason, it was inactive initially as expressed in E. coli. Other approaches, such as co-expression with stabilizing binding partners or expression in P. aeruginosa, respectively, may also be useful.

Alternative approach: Co-expression of DnaX, HolA and HolB. The primary approach that involved expression of all three proteins (DnaX, HolA and HolB) separately was presented because it is considered to be the most reliable, but a ‘short-cut’ in parallel with the primary approach is also contemplated. In E. coli, DnaX, HolA and HolB interact synergistically to form an isolable complex. The genes for the E. coli DnaX complex can be coexpressed, and a complex of all proteins isolated in a single purification. P. aeruginosa DnaX, HolB and HolA will be combined into an artificial operon and it will be determined whether an isolable complex forms that can be purified as one entity. The purification will be monitored by reconstitution assays with DnaE and DnaN, just as in the E. coli system (Pritchard, A., Dallmann, G., Glover, B., and McHenry, C. (2000) EMBO J 19, 6536-6545; Pritchard, A. E., Dallmann, H. G., and McHenry, C. S. (1996) J Biol Chem 271, 10291-10298). This approach, if successful, would greatly accelerate progress and expedite the reconstitution of a purified P. aeruginosa replicase using purified proteins.

Accessory Proteins

### Example 53

### Molecular Cloning of P. aeruginosa dnaG, the Gene Encoding DnaG (Primase)

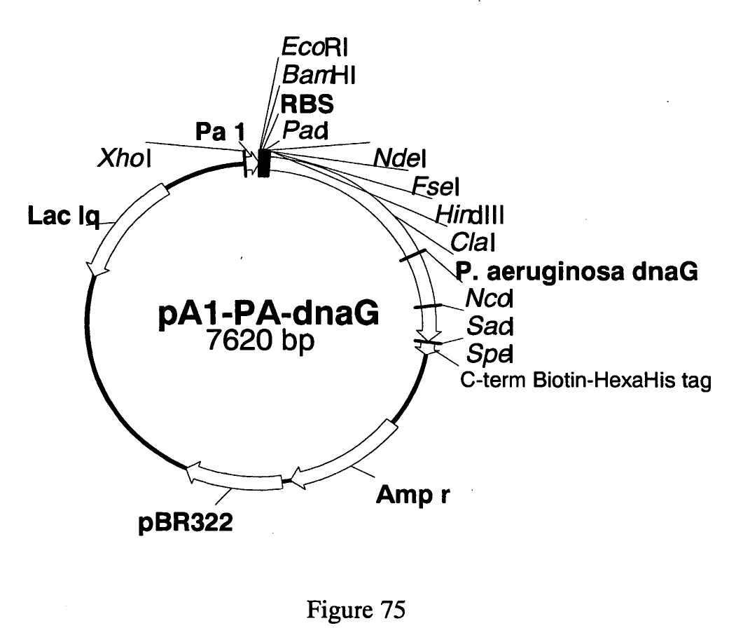

This example describes PCR amplifying the dnaG gene, which encodes the DnaG primase, from P. aeruginosa genomic DNA. The dnaG gene will then be inserted into the pA1-CB-NdeI plasmid (polyclonal region of pA1-CB-NdeI is shown in FIG. 31). The dnaG gene does not have any 5′ low-usage codon for expression in E. coli. The DnaG primase is composed of 664 amino acids. The gene encoding the DnaG primase is dnaG, and is composed of 1995 nucleotides including the stop codon. The nucleotide sequence of dnaG is represented by (SEQ ID NO:102). The amino acid sequence of DnaG primase is represented by SEQ ID NO:104. To PCR amplify the dnaG gene, the forward/sense primer is: 5′-gactCATATGGCCGGCCTGATACCGCAAA-3′ (29 mer) (SEQ ID NO:105). There is a 4-nucleotide 5′ clamp (lower case-gact) allowing efficient digestion by the restriction enzyme. The clamp is followed by an NdeI restriction site (uppercase, italic letters), which allows insertion into pA1-CB-NdeI at optimal spacing from the RBS. The NdeI restriction site overlaps the “atg” start codon of the dnaG gene. The next 19 nucleotides correspond to positions 4-22 of the 5′ end of the dnaG gene and are shown as upper case and underlined. The reverse/antisense primer is: 5′-gactACTAGTtcaTCAGCTCTGGGAAGGCGATGAA-3′ (35 mer) (SEQ ID NO:106). There is a 4-nucleotide 5′ clamp (lower case-gact) allowing efficient digestion by the restriction enzyme. The clamp is followed by a SpeI restriction site (uppercase, italic letters), which allows insertion into pA1-CB-NdeI. Next, there is a second non-complementary stop codon, which is adjacent to the native stop codon. The final 22 nucleotides are complementary to the 3′ end of the dnaG gene and are shown in uppercase and underlined letters. The PCR product is digested with NdeI/SpeI restriction enzymes. The fragment containing the dnaG gene (2.0 kb) is inserted into pA1-CB-NdeI that has been digested with the same two restriction enzymes. This results in the plasmid pA1-PA-dnaG, which contains the full length P. aeruginosa dnaG gene, and is expressed as a native protein. pA1-PA-dnaG is graphically shown in FIG. 75.

### Example 54

### Molecular Cloning of P. aeruginosa ssb, the Gene Encoding SSB

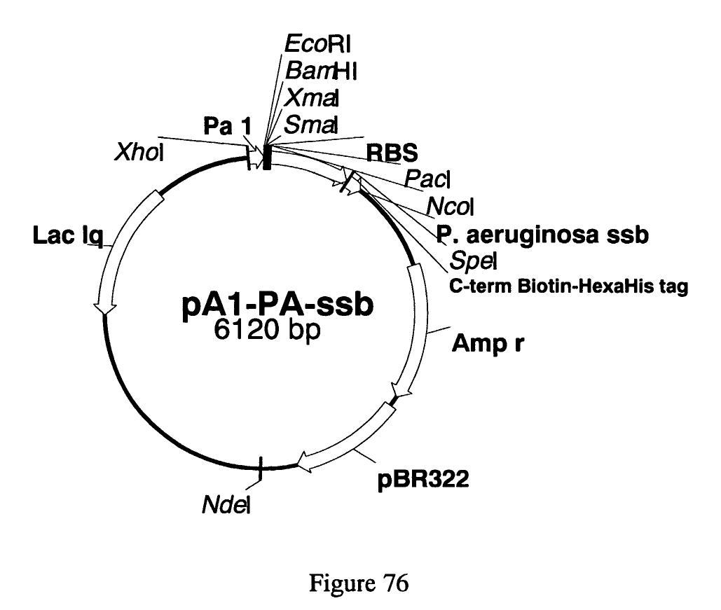

This example describes PCR amplifying the ssb gene, which encodes SSB, from P. aeruginosa genomic DNA. The ssb gene is then inserted into the pA1-CB-NcoI plasmid (polyclonal region of pA1-CB-NcoI is shown in FIG. 31). The ssb gene does not have any 5′ low-usage codon for expression in E. coli. The SSB protein is composed of 165 amino acids. The gene encoding the SSB protein is ssb, and is composed of 498 nucleotides including the stop codon. The nucleotide sequence of ssb is represented by SEQ ID NO:107. The amino acid sequence of SSB is represented by SEQ ID NO:109. To PCR amplify the ssb gene, the forward/sense primer is: 5′-gatcCCATGGCCCGTGGGGTTAACAAA-3′ (27 mer) (SEQ ID NO:110). There is a 4-nucleotide 5′ clamp (lower case-gatc) allowing efficient digestion by the restriction enzyme. The clamp is followed by an NcoI restriction site (upper-case, italics), which allows insertion into pA1-CB-NcoI and at optimal spacing from the RBS. The NcoI restriction site overlaps the first 4 nucleotides of the 5′ end of the ssb gene. The next 17 nucleotides correspond to nucleotides 5-21 of the 5′ end of the ssb gene. All nucleotides corresponding to the 5′ end of the ssb gene are upper case and underlined. The reverse/antisense primer is: 5′-gactACTAGTtcaTTAGAACGGAATGTCGTCGTCGAA-3′ (37 mer) (SEQ ID NO:111). There is a 4-nucleotide 5′ clamp (lowercase gact) allowing for efficient digestion by the restriction enzyme. Following the clamp is a SpeI restriction site (uppercase, italic letters), which is used for insertion into pA1-CB-NcoI. Next, is a second non-complementary stop codon that is adjacent to the native stop codon (lowercase, boldface letters). The final 24 nucleotides are complementary to the 3′ end of the ssb gene. The PCR product is digested with NcoI/SpeI restriction enzymes. The fragment containing the ssb gene (approximately 0.5 kb) is inserted into pA1-CB-NcoI that has been digested with the same two restriction enzymes. This results in the plasmid pA1-PA-ssb, which contains the full length P. aeruginosa ssb gene, which is expressed as a native protein. pA1-PA-ssb is graphically shown in FIG. 76.

Reconstitution

### Example 55

### Reconstitution of the Minimal DNA Pol III Holoenzyme From P. aeruginosa Consisting of α, β, DnaX, δ and δ′ Subunits and Adaption of the Assay to the Multiplicative Target Screening™ Assay

The minimal assembly of the essential subunits of a P. aeruginosa replicase should permit rapid and processive synthesis of long stretches of DNA. In all bacterial replicases studied so far, the minimal functional holoenzyme consists of three components: 1) the polymerase core, 2) the clamp loading assembly and 3) the sliding clamp processivity factor. In E. coli, the minimal holoenzyme consists of the a subunit, DnaX-δδ′ clamp loading complex and the β subunit. The same components are minimally required for functional DNA Pol III holoenzyme from Gram-positive S. pyogenes (Bruck & Donnell, ibid.) and Thermus thermophilus (Bullard, et al., ibid.). Given the generality of this requirement among distantly related organisms, it is expected that P. aeruginosa α, β, DnaX, δ, and δ′ will be the only proteins minimally required. The backup approach presented with regard to the DnaX complex can be utilized to provide additional proteins, in the unlikely event they are minimally required.

With the availability of the essential P. aeruginosa DNA polymerase III holoenzyme subunits purified by a reconstitution approach, the fluorescence-based Multiplicative Target Screen™ assay will be adapted to permit a high-throughput screen against the new target provided. This will be done by titrating each component until it is barely limiting so that inhibition will be observed upon interaction with a ‘hit’ with a target. Buffer conditions will also be optimized to ensure stability of the enzyme mix, stored in a separate chilled container, during the multi-plate screening process. The time and temperature of the assay will also be reoptimized to ensure a stable linear response. For the initial assays, a DNA primer will be annealed to an M13 circle, bypassing to requirement for the initial enzyme-catalyzed priming step (FIG. 1). This accomplishment will establish the feasibility of assembling a more complete P. aeruginosa replication system and provide the foundation for screening libraries of small molecules (obtained from several suppliers of combinatorial chemistry libraries) in a high-throughput format to identify inhibitors of the P. aeruginosa replicative polymerase.

### Example 56

### Complete Replication System for P. aeruginosa

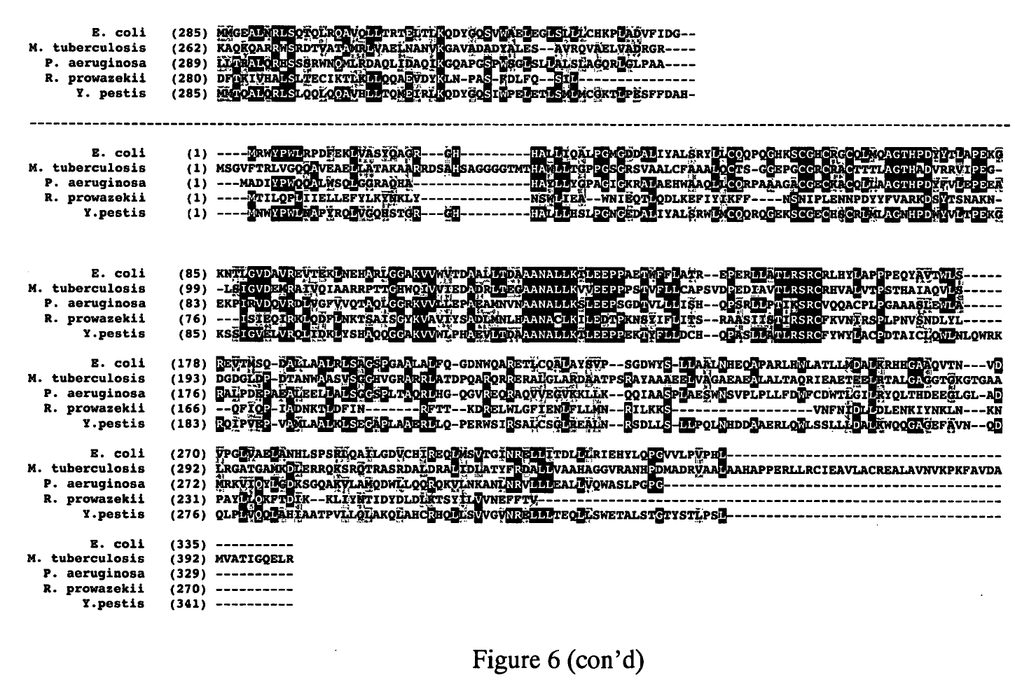

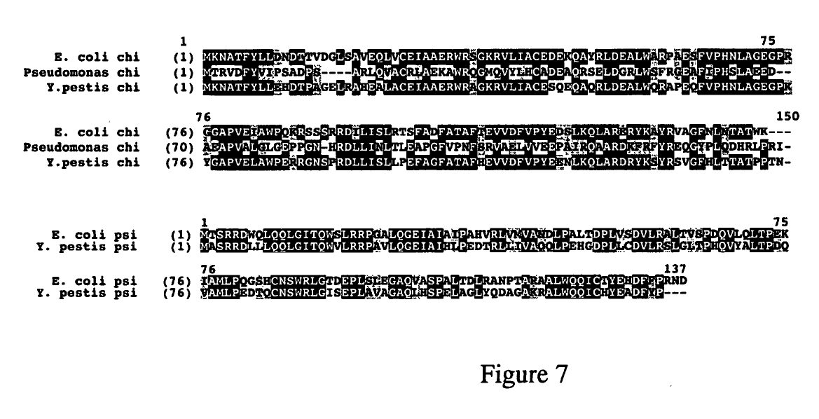

It will be possible to provide a more complete replication system from Psudomonas aeruginosa by further expressing and purifying (i) DnaQ (the 3′-5′ proofreading exonuclease that is expected to be associated with DnaE), (ii) HolC (ψ subunit), HolD (χ subunit) and SSB (which provide an additional physical and functional link of the holoenzyme to DNA, facilitating replication, particularly at physiological ionic strength) (FIGS. 7, 8), (iii) DnaG primase that may directly recognize the G4 origin of M13Gori and synthesize a primer (FIG. 8) and (iv) the DnaB helicase (by analogy to E. coli, DnaB should recognize single-stranded DNA, interact with the DnaG primase and enable primer synthesis providing a ‘backup’ approach if DnaG cannot directly recognize the G4 origin) (FIG. 8).

At this point libraries of small molecules (obtained from several suppliers of combinatorial chemistry libraries) will be screened in a high-throughput format to identify inhibitors of the P. aeruginosa replicative polymerase. With replication systems from multiple pathogens in hand, a method of identifying both narrow- and broad-spectrum antibacterial drugs will comprise setting up parallel screens against replication systems from multiple organisms. Importantly, since the replication system represents a novel target for antibacterial drugs, resistance mechanisms should not yet exist.

1. A method of screening for a compound that modulates the activity of a DNA polymerase III replicase, said method comprising:
a) contacting an isolated replicase with at least one test compound under conditions permissive for replicase activity;
b) assessing the activity of the replicase in the presence of the test compound; and
c) comparing the activity of the replicase in the presence of the test compound with the activity of the replicase in the absence of the test compound, wherein a change in the activity of the replicase in the presence of the test compound is indicative of a compound that modulates the activity of the replicase,
wherein said replicase comprises an isolated Y. pestis DNA polymerase III subunit protein.

2. The method of claim 1, wherein said isolated Y. pestis DNA polymerase III subunit protein is encoded by a nucleic acid molecule selected from the group consisting of
a) SEQ ID NO:1, SEQ ID NO:8, SEQ ID NO:13, SEQ ID NO:18, SEQ ID NO:23, SEQ ID NO:33, SEQ ID NO:38, SEQ ID NO:43, SEQ ID NO:48, SEQ ID NO:53, and SEQ ID NO:58; and
b) a protein comprising a homologue of a protein of a), wherein said homologue encodes a protein containing one or more amino acid deletions, substitutions, or insertions, and wherein said protein performs the function of a natural subunit protein in a bacterial replication assay; and
c) an isolated bacterial nucleic acid molecule which is fully complementary to any said nucleic acid molecule recited in a).

3. (cancelled).

4. A method of identifying compounds which modulate the activity of a DNA polymerase III replicase comprising:
a) forming a reaction mixture that includes a DNA molecule, a DNA polymerase α subunit, a candidate compound, a dNTP, and optionally, a member of the group consisting of a β subunit, a τ complex, and both the β subunit and the τ complex, to form a replicase;
b) subjecting the reaction mixture to conditions effective to achieve nucleic acid polymerization in the absence of the candidate compound; and
c) comparing the activity of the replicase in the presence of the test compound with the activity of the replicase in the absence of the test compound, wherein a change in the activity of the replicase in the presence of the test compound is indicative of a compound that modulates the activity of the replicase,
wherein said replicase comprises a Y. pestis DNA polymerase III subunit protein.

5. The method of claim 4, wherein said isolated Y. pestis DNA polymerase III subunit protein is encoded by a nucleic acid molecule selected from the group consisting of
a) SEQ ID NO:1, SEQ ID NO:8, SEQ ID NO:13, SEQ ID NO:18, SEQ ID NO:23, SEQ ID NO:33, SEQ ID NO:38, SEQ ID NO:43, SEQ ID NO:48, SEQ ID NO:53, and SEQ ID NO:58; and
b) a protein comprising a homologue of a protein of a), wherein said homologue encodes a protein containing one or more amino acid deletions, substitutions, or insertions, and wherein said protein performs the function of a natural subunit protein in a bacterial replication assay; and
c) an isolated bacterial nucleic acid molecule which is fully complementary to any said nucleic acid molecule recited in a).

6.-199. (cancelled).
